(19) United States
(12) Patent Application Publication
(10) Pub. No.: US 2025/0346782 A1
(43) Pub. Date: Nov. 13, 2025

(54) CHEMICAL ENTITIES

(71) Applicant: W. L. Gore & Associates, Inc., Newark, DE (US)

(72) Inventors: Charles D. Claude, Newark, DE (US); Paul D. DRUMHELLER, Newark, DE (US); Christopher Kuehl, Newark, DE (US)

(73) Assignee: W. L. Gore & Associates, Inc., Newark, DE (US)

(21) Appl. No.: 18/856,754

(22) PCT Filed: Apr. 14, 2023
(86) PCT No.: PCT/US23/18695

§ 371 Date: Oct. 14, 2024

Related U.S. Application Data
(60) Provisional application No. 63/331,158, filed on Apr. 14, 2022.

(51) Int. Cl. C09D 183/08; C07C 323/41; C07C 323/42; C07D 295/185

(52) U.S. Cl. CPC C09D 183/08; C07C 323/41; C07C 323/42; C07D 295/185

(57) ABSTRACT

Embodiments of the present disclosure relate to a chemical entity formed by reaction of components B and C, wherein component C is a moiety comprising one or more amine groups, and component B is a compound of Formula (I): wherein, A is a core moiety; L is an optional linker; R¹ and R² are independently selected from H and C₁₋₅ alkyl; or R¹ and R² together with the C atom to which they are attached combine to form a C₄₋₇ cycloalkyl ring or a 4-7 membered heterocyclic ring, both of which are optionally substituted on an available atom by one or more groups selected from C₁₋₂ alkyl and oxo; n Sis O or 1; and m is an integer of at least 1. In one embodiment, at least a proportion of the thiol (—SH) groups in the chemical entity have been converted to S-nitrosothiol (—SNO) groups.

### FIELD

Embodiments of the present disclosure relate to chemical entities capable of releasing nitric oxide. The chemical entities are of use in coatings for surfaces, in particular for the surfaces of medical devices. Methods for making such chemical entities and coatings are also described.

### BACKGROUND

It has become increasingly common to treat a variety of medical conditions by introducing a medical device into an organ or tissue within the body. Such medical devices may be transiently or permanently implanted, into tissues or organs as bone, nerve, brain, smooth muscle, cardiac muscle, kidney, lung, liver, stomach, intestine, uterus, vagina, prostate, testicles, and the like. Many implants are prosthetics, intended to replace missing body parts, while others perform important functions such as delivering medication, monitoring bodily functions, or providing support to organs and tissues.

For example, medical devices used for the treatment of vascular disease include stents, stent-grafts, grafts, catheters, balloon catheters, guide wires, cannulas and the like. Such devices may be implantable, e.g. into a blood vessel, and typically provide a medical benefit by mechanical action (e.g. by expanding and/or supporting the walls of a blood vessel), but may also be drug-eluting to provide additional medical benefits (e.g. eluting an antiproliferative compound such as paclitaxel to prevent restenosis). Several implantable devices for localized drug delivery are known, including a stent coated with an elutable drug, also known as a drug eluting stent (DES). Medical devices that are introduced into the vascular system for a transient length of time, may also be drug-coated, e.g. a balloon catheter coated with a drug, also known as a drug coated balloon (DCB), or a catheter coated with a drug, also known as a drug coated catheter (DCC).

However, the use of such medical devices can be associated with problems well known in the art, including early and late thrombosis associated with vascular inflammation, formation of mural emboli, anastomotic hyperplasia, endoleak, vascular constriction, and the like. It is also well known that when a medical device is contacted with blood, plasma proteins adsorb to the device surface. These adsorbed plasma proteins facilitate attachment of platelets which, upon attachment, directly interact with said plasma proteins to expose the platelet glycoprotein GPIIb/IIIa integrin receptor which facilitates binding to fibrinogen (Calvete et al., 1995). A positive-feedback loop of platelet activation and aggregation ensues, culminating in clot formation. There is a secondary risk of this clot detaching and causing embolism elsewhere in the vasculature.

In addition, certain implantable medical devices can be a major source of infection, particularly those which are implanted for longer periods of time. Such infections can be associated with early device failures, requiring repeated surgical procedures. Transcutaneous devices such as transcutaneous catheters, pacing leads, colon stoma, cranial shunts, and the like have an increased risk of infection, leading to exacerbated patient discomfort when such devices are regularly removed and replaced to avoid such infection.

Nitric oxide (NO) is a signalling molecule produced by nitric oxide synthase processing of L-arginine. Endothelial cells (ECs) are a producer of NO and are the main source of NO production in the vascular system. In this context, NO is important in maintaining cardiovascular homeostasis and regulating vasodilation. Furthermore, NO has other actions, such as potently inhibiting platelet adhesion and aggregation; preventing thrombosis; inhibiting proliferation of inflammatory cells, and inhibiting smooth muscle cell proliferation, promoting growth and adhesion of endothelial cells; and inhibiting leukocyte activation. As such, NO has been demonstrated to improve the biocompatibility of medical devices (Frost et al., 2005). Similarly, NO has been implicated the treatment of atherosclerosis (Matthys and Bult, 1997). In addition, NO is an inhibitor of bacterial adhesion and proliferation (Schairer et al., 2012) and inducible nitric oxide synthase is expressed by T cells, macrophages, and mature dendritic cells, and regulates the differentiation and function of immune cells via nitration of key molecules involved in transcriptional or signalling pathways (Xue et al., 2018). NO plays an important role in the mobilization, differentiation, and function of endothelial progenitor cells. As such, NO has been implicated in the regulation of endothelialization and angiogenesis (Cooke and Losordo, 2002).

US2015/0247005 discloses stable, photosensitive polymers that release NO in response to the intensity and wavelength of light.

US2017/0246353 discloses a method for producing a nitric oxide-generating coating comprising preparing a buffer solution containing polyphenol compounds, organic selenium or sulphur compounds and soluble copper salts; then contacting a base material with the solution, and washing and drying to obtain a product. Organic selenium and organic sulphur compounds are said to have glutathione peroxidase-like activity, that is they can catalyse nitrosothiol to release NO.

US2019/0358368 discloses tubing impregnated with a silicone oil and a NO-releasing agent which consequently has anti-fouling characteristics, that is to say the tubing has a reduced tendency to attach platelets, induce thrombosis, or facilitate infection. Methods of preparing such tubing and methods for delivering a pharmaceutically acceptable fluid via the tubing are also disclosed.

WO2020/018488 discloses NO-releasing materials, methods of preparing said materials and devices including said materials. Said NO-releasing material includes a polymer matrix comprising a plurality of polysiloxanes, amine-containing crosslinkers, and NO-donating moieties such as S-nitrosothiol moieties derived from thiols such as tertiary thiols, wherein the NO-donating moieties are reacted with the amine-containing crosslinker. The methods and materials described are said to prevent thrombosis and biofilm formation.

WO2021/126084A1 discloses a composite material comprising a substrate coated with a block copolymer brush, where the block copolymer brush comprises a first block of a hydrophobic polymer conjugated to a nitric oxide source, where the first block of the hydrophobic polymer is covalently bonded to a surface of the substrate or a first block of a cationic polymer covalently bonded to a surface of the substrate and a second block of a hydrophilic polymer, extending from the first block to form an outer surface of the block copolymer brush.

It is known to the art that peptide sequences can comprise thiols along the peptide chain backbone in the form of pendant tertiary thiols. For example, a first peptide can be ligated at the C-terminus with penicillamine thiolactone to produce a peptide comprising an electrophilic thioester, and a second peptide can be modified with an N-terminal cysteine to produce a peptide comprising a nucleophilic tertiary thiol (Chen et al., 2018). In another example, a first peptide can be modified to produce a peptide comprising an electrophilic thioester, and a second peptide can be ligated with penicillamine to produce a peptide comprising a nucleophilic tertiary thiol (Altenbruun et al., 2009). In both teachings, the first peptide can thus be condensed via transthioesterification with the second peptide to produce an intermediate dipeptide conjugate, which undergoes a chemoselective S-to-N acyl shift (i.e., the well-known native chemical ligation reaction), to produce a final dipeptide conjugate comprising a pendant tertiary thiol along the backbone of the dipeptide conjugate. However, such dipeptide conjugates are not suitable for derivatization to pendant NO-donating moieties such as S-nitrosothiol moieties, for use in coatings for surfaces, in particular for the surfaces of medical devices for use in treating tissue in the human or animal body. It is well known that peptide coatings are enzymatically labile when implanted in a human or animal body. Peptide coatings comprising a pendant tertiary thiol along the peptide backbone would therefore be subject to enzymatic degradation which can lead to desorption, flaking, leaching, or other damage to the coating, and are thus unsuitable as a coating for releasing nitric oxide.

There is a need to develop further NO-releasing coatings, especially for use on devices that are introduced into the human body, e.g. in the localized treatment of vascular disease. In particular, there is a need to develop coatings for medical devices comprising NO that can deliver therapeutically relevant levels of NO to a target tissue (such as vascular tissue), in a localised manner, on a suitable timescale. When the medical device has a coating with an additional therapeutic agent (i.e. other than NO), the NO-releasing coating should be compatible with the additional therapeutic agent.

Such coatings are also potentially of use for ex vivo or extracorporeal medical devices such as a blood oxygenator, a dialysis machine, and the like.

### SUMMARY

Embodiments of the present disclosure relate to novel chemical entities that are capable of releasing nitric oxide. When coated onto a surface, in particular a surface of a medical device, such coatings may be of use in the treatment of a condition that benefits from the release of nitric oxide.

Thus, in one embodiment is provided a chemical entity formed by reaction of components B and C, wherein component B is a compound of Formula (I):

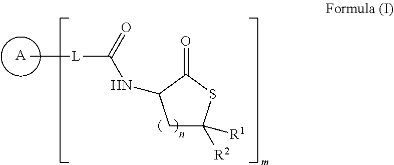

wherein,
A is a core moiety;
L is an optional linker;
R¹ and R² are independently selected from H and C₁₋₅ alkyl; or R¹ and R² together with the C atom to which they are attached combine to form a C₄₋₇ cycloalkyl ring or a 4-7 membered heterocyclic ring, both of which are optionally substituted on an available atom by one or more groups selected from C₁₂ alkyl and oxo;
n is 0 or 1; and
m is an integer of at least 1;
and,
component C is a moiety comprising one or more amine groups.

In another embodiment is provided a surface having a coating comprising a chemical entity formed by reaction of components B and C.

The reaction of components B and C results in the ring opening of the thiolactone ring and leads to the formation of a chemical entity comprising one or more SH groups. In another embodiment is provided a chemical entity and a surface having a coating comprising a chemical entity, wherein at least a proportion of the —SH groups in the chemical entity have been converted to —SNO (S-nitrosothiol) groups. Such surfaces can form at least a part of a surface of medical device, which can be of use in the treatment or prevention of a condition which benefits from the delivery of nitric oxide.

### BRIEF DESCRIPTION OF THE FIGURES

FIG. 1A shows Embodiment 1, wherein for component B m is 1 and component C comprises one amine group; Embodiment 2, wherein for component B m is 1 and component C comprises two amine groups; and Embodiment 3 wherein for component B m is 2 and component C comprises one amine group.

FIG. 1B shows Embodiment 4, wherein for component B m is 2 and component C comprises two amine groups.

FIG. 1C shows Embodiment 5, wherein for component B m is 2 and component C is a dendrimer (PAMAM-GO).

FIG. 1D shows Embodiment 6, wherein for component B m is 4 and component C comprises two amine groups.

FIG. 1E shows Embodiment 7, wherein for component B m is 2, and component C is a polyamine (branched PEI).

FIG. 2 shows the ¹H NMR (DMSO-d6) spectrum of copolymer (19) (Example 17).

FIG. 3 shows the overlayed FTIR spectra of copolymers (19) and (20) (Example 23).

FIG. 4 shows the NO release profile for an ePTFE film coated with compound (16) (Example 25).

FIG. 5 shows the NO release profile for an ePTFE film coated with compound (17) (Example 25).

FIG. 6 shows the NO release profile for an ePTFE film coated with VDF-HFP fluoropolymer and compound (16) (Example 26).

FIG. 7 shows the NO release profile for an ePTFE film coated with VDF-HFP fluoropolymer and compound (18) (Example 26).

FIG. 8 shows the NO release profile for stainless steel foil coated with VDF-HFP fluoropolymer and compound (18) (Example 27).

FIG. 9 shows the NO release profile for Pebax® nylon coated with VDF-HFP fluoropolymer and compound (18) (Example 28).

FIG. 10 shows the NO release profile for ePE coated with VDF-HFP fluoropolymer and compound (18) (Example 29).

FIG. 11 shows the NO release profile for an ePTFE film coated with VDF-HFP fluoropolymer and copolymer (20) (Example 26).

FIG. 12 shows the NO release profile for an ePTFE film coated with polymer (22) (Example 30).

FIG. 13 is a gel permeation chromatography (GPC)—light scattering (LS) plot for Polymer (32) of Example A2 (here designated DDPP) obtained using refractive index and light scattering detectors and showing average Mₙ, M_{w}, M_{z} and M_{w}/Mₙ values for the polymer.

FIG. 14 shows the FT-IR spectra of non-nitrosylated PPO-400 dimethylmalonyl penicillamine polymer (33) of Example A3 and nitrosylated PPO-400 dimethylmalonyl penicillamine polymer (37) of Example A5. The dashed line is the spectrum of the non-nitrosylated polymer (33) and the solid line is the spectrum of the nitrosylated polymer (37).

FIG. 15 is a TGA plot for the non-nitrosylated PPO-400 dimethylmalonyl penicillamine polymer (33) of Example A3 and nitrosylated PPO-400 dimethylmalonyl penicillamine polymer (37) of Example A5. The upper (solid) line represents the non-nitrosylated polymer (33) and the lower (dashed) line represents the nitrosylated polymer (37).

FIG. 16 is a plot showing the NO release profile from nitrosylated PPO-400 dimethylmalonyl penicillamine polymer (37) over a period of 648 hours (27 days), measured using the Griess reagent NO elution test method.

FIG. 17 is a series of SEM images showing the surfaces of vascular grafts which are either uncoated or coated with the nitrosylated PPO-400 dimethylmalonyl penicillamine polymer (37) of Example A5. 17a: uncoated, 50× magnification; 17b: coated, 50× magnification; 17c: uncoated, 2000× magnification; 17d: coated 2000× magnification.

FIG. 18 is a series of SEM images showing the surface of a vascular graft coated with the nitrosylated PPO-400 dimethylmalonyl penicillamine polymer (37) of Example A5 after passing round a stainless steel cone with an outer diameter of 15 mm as specified in ISO 7198.

FIG. 19 is an ESI mass spectrum of Compound 14 showing peaks for M+H⁺ and M+Na⁺.

FIG. 20 is an ESI mass spectrum of Compound 14 following fragmentation into the thiolactone and amine starting materials and showing peaks corresponding to M+H⁺ for the compound, for fragments which have lost one or two amine fragments and for the amine fragment.

FIG. 21 is an ESI mass spectrum of Compound 38 showing peaks for M+H⁺ and M+Na⁺.

FIG. 22 is an ESI mass spectrum of Compound 38 following fragmentation and loss of one thiolactone fragment.

FIG. 23 is an ESI mass spectrum of Compound (38) following fragmentation and shows peaks corresponding to M+H⁺ for a fragment which has lost a thiolactone fragment, for the thiolactone fragment and for the remaining amine fragment after loss of both thiolactone fragments.

### DETAILED DESCRIPTION

The present disclosure relates to novel thiol-containing and/or S-nitrosothiol-containing chemical entities, and to surfaces having coatings comprising such chemical entities.

Chemical entities of the disclosure may be in “thiol form/—SH form” (containing at least a proportion of thiol groups) or in “S-nitrosothiol/—SNO form” (containing at least a proportion of S-nitrosothiol groups).

As is known the art, chemical entities containing S-nitrosothiol groups are capable of releasing nitric oxide. When the chemical entities are applied as coatings on a surface, in particular the surface of a medical device, the resulting coatings are capable of releasing nitric oxide.

The thiol form of a chemical entity of the disclosure is prepared by reaction of components B and C. In this context and in the context of the claims, the term “chemical entity” is intended to cover all possible moieties resulting from the reaction of component B with component C, e.g. a compound, copolymer or polymer.

Component B comprises a core moiety A bound to at least one cyclic thioester moiety. Component C is a moiety comprising one or more amine groups. When components B and C react together, the amine group(s) of component C react with the cyclic thioester moiety/moieties of component B via an aminolysis reaction, to form one or more secondary amide linkers, and one or more free thiol (—SH) groups. Depending on the nature of groups R¹ and R², the thiol will be a primary thiol, a secondary thiol or a tertiary thiol. Depending on the nature of components B and C, the chemical entity can be further defined as a compound, a copolymer or a polymer.

Chemical entities of the disclosure comprise moieties that are chiral, and may comprise a D-stereoisomer, an L-stereoisomer, or a mixture (such as a racemic mixture) of the two. In embodiments where the chemical entity contains at least two chiral centres, the chemical entity may be chiral or non-chiral (meso). In one embodiment, the chemical entity may be bound to a medical device, and may comprise one or more D-stereoisomers, one or more L-stereoisomers, or a racemic mixture of stereoisomers. In another embodiment, the chemical entity may be eluted from a medical device, and may comprise a specific stereoisomer or a specific racemic mixture selected to maximize therapeutic effect.

### Component B

Component B is a compound of Formula (I):

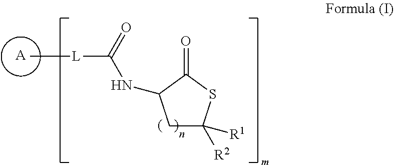

wherein,
A is a core moiety;
L is an optional linker;
R¹ and R² are independently selected from H and C₁₋₅ alkyl; or R¹ and R² together with the C atom to which they are attached combine to form a C₄₋₇ cycloalkyl ring or a 4-7 membered heterocyclic ring, both of which are optionally substituted on an available atom by one or more groups selected from C₁₋₂ alkyl and oxo;
n is 0 or 1; and
m is an integer of at least 1.

In one embodiment, core moiety A comprises one or more moieties independently selected from the group consisting of alkyl, spiroalkyl, aryl, heteroaryl, alkyl-aryl, a porphyrin, a polymer and a macrocycle, wherein alkyl, spiroalkyl, aryl, heteroaryl and alkyl-aryl are optionally substituted (e.g. by one or more substituents selected from the group consisting of alkyl, alkoxy, fluoroalkyl and fluoroalkoxy).

When core moiety A comprises or is an alkyl group (e.g. C₁₋₂₀ alkyl, C₁₋₁₀ alkyl, C₁₋₆ alkyl, C₁₋₄ alkyl or C₁₋₃ alkyl (e.g. methyl or ethyl)), it is optionally substituted by one or more substituents (e.g. selected from the group consisting of alkyl, alkoxy, fluoroalkyl and fluoroalkoxy e.g. C₁₋₆ alkyl, C₁₋₆ alkoxy, C₁₋₆ fluoroalkyl and C₁₋₆ fluoroalkoxy, or C₁₋₄ alkyl, C₁₋₄ alkoxy, C₁₋₄ fluoroalkyl and C₁₋₄ fluoroalkoxy). The alkyl group can be branched or unbranched. In one embodiment, core moiety A is an alkyl group.

In some cases when the core moiety A comprises an alkyl group or is an alkyl group and m is 1 or 2, especially 2. In some suitable compounds, m is 2 and the core moiety A comprises or consists of a C₁₋₁₀ alkylene moiety, for example a C₁₋₆ alkylene moiety and especially a C₁₋₄ alkylene moiety such as —(CH₂)₄—, —(CH₂)₃—, —C(CH₃)₂—, —CH(CH₃)—, —CH₂—, —CH(CH₃)CH₂—, —CH₂CH(CH₃)— or —CH₂CH₂—, more especially, C₂₋₄ alkylene such as —(CH₂)₄—, —(CH₂)₃—, —C(CH₃)₂—, —CH(CH₃)—, —CH(CH₃)CH₂—, —CH₂CH(CH₃)— or —CH₂CH₂—. In an embodiment, A is —C(CH₃)₂— and m is 2. In another embodiments, A is —(CH₂)₄— and m is 2. In a suitable embodiment, the linker L is absent.

When core moiety A comprises a spiroalkyl group (e.g. C₁₋₂₀ spiroalkyl, C₁₋₁₀ spiroalkyl or C₁₋₆ spiroalkyl) it is optionally substituted by one or more substituents (e.g. selected from the group consisting of alkyl, alkoxy, fluoroalkyl and fluoroalkoxy). In one embodiment, core moiety A is a spiroalkyl group.

When core moiety A comprises an aryl group (e.g. 5-20 membered aryl, 5-12 membered aryl, 5-10 membered aryl or 5-7 membered aryl) it is optionally substituted by one or more substituents (e.g. selected from the group consisting of alkyl, alkoxy, fluoroalkyl and fluoroalkoxy). “Aryl” as used herein is a cyclic group with aromatic character, such as phenyl or naphthyl. Aryl also includes diaryl and polyaryl groups. In a suitable embodiment, aryl is phenyl, which is optionally substituted by one or more substituents selected from the group consisting of alkyl, alkoxy, fluoroalkyl and fluoroalkoxy. In one embodiment, core moiety A is an aryl group.

In one embodiment, m is 2 and core moiety A is:

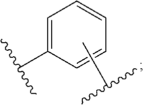

m is 3 and core moiety A is:

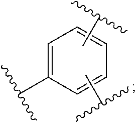

for example, m is 2 and core moiety A is:

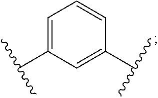 [SMILES: *c1cccc(*)c1]

m is 3 and core moiety A is:

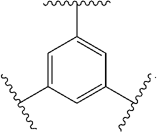 [SMILES: *c1cc(*)cc(*)c1]

When core moiety A comprises an heteroaryl group, it is optionally substituted by one or more substituents (e.g. selected from the group consisting of alkyl, alkoxy, fluoroalkyl and fluoroalkoxy). In one embodiment, heteroaryl is 5-10 membered heteroaryl, e.g. pyrrolyl, furanyl, thienyl, imidazolyl, pyrazolyl, thiazolyl, isothiazolyl, oxadiazolyl, thiadiazolyl, triazolyl, oxazolyl, isoxazolyl, tetrazolyl, pyridinyl, pyrimidinyl, pyradizinyl or pyrazinyl. In one embodiment, core moiety A is an heteroaryl group.

When core moiety A comprises an alkyl-aryl group (e.g. aryl substituted by alkyl (e.g. C₁₋₁₀ alkyl) or polyaryl linked by alkyl (e.g. C₁₋₅ alkyl)), it is optionally substituted by one or more substituents (e.g. selected from the group consisting of alkyl, alkoxy, fluoroalkyl and fluoroalkoxy). In one embodiment, core moiety A is an alkyl-aryl group.

In one embodiment, core moiety A comprises a porphyrin e.g. tetra-4-aryl-meso. In one embodiment, core moiety A is a porphyrin.

In one embodiment, core moiety A comprises a polymer (e.g. a polyamine compound, a dendrimer or a hyperbranched polymer). In one embodiment, the polyamine compound is selected from the group consisting of polyethyleneimine, polyallylamine, polylysine, polyarginine and polyaminosilane. In one embodiment, core moiety A comprises polyethyleneimine (PEI), in particular branched polyethyleneimine. Suitably, the polyethyleneimine (in particular branched polyethyleneimine) has molecular weight of 10-1,000 kDa e.g. 10-50 kDa or 50-100 kDa. In one embodiment, core moiety A comprises a dendrimer, e.g. a polyamine dendrimer such as a PAMAM dendrimer or a PPI-dendrimer. In one embodiment, core moiety A is a polymer.

In one embodiment, core moiety A comprises a macrocycle, e.g. a cyclodextrin. In one embodiment, core moiety A is a macrocycle.

In one embodiment, linker L comprises C₁₋₁₀ alkylene, a secondary amine or an amide. In another embodiment, linker L is absent.

In one embodiment, n is 0. In another embodiment, n is 1. Suitably, n is 0.

In one embodiment, R¹ and R² are independently selected from the group consisting of H and C₁₋₅ alkyl. In another embodiment, R¹ and R² are independently selected from the group consisting of CH₂CH₃ and CH₃; and in particular are both CH₃. In another embodiment, R¹ and R² together with the C atom to which they are attached combine to form a C₄₋₇ cycloalkyl ring or a 4-7 membered heterocyclic ring, both of which are optionally substituted on an available atom by one or more groups selected from C₁₋₂ alkyl and oxo; e.g. R¹ and R² together with the C atom to which they are attached combine to form a cycloheptyl or cyclohexyl ring which is optionally substituted on an available atom by one or more groups selected from C₁₋₂ alkyl and oxo.

As used herein, the term “oxo” refers to a ═O substituent, whereby an oxygen atom is doubly bonded to carbon (e.g. C═O) or another element (e.g. S═O, S(═O)₂).

The term C₄₋₇ cycloalkyl ring (such as C₄₋₅ cycloalkyl, C₄₋₆ cycloalkyl or C₅₋₆ cycloalkyl) refers to a fully saturated cyclic hydrocarbon group having from 4 to 7 carbon atoms. The term encompasses cyclobutyl, cyclopentyl and cyclohexyl.

The term “4-7 membered heterocyclic ring” refers to a non-aromatic cyclic group having 4 to 7 ring atoms, at least one of which is a heteroatom selected from N, O, S and B, in particular selected from N, O and S, e.g. selected from N and O. The term “heterocyclic ring” is interchangeable with “heterocyclyl”. The term encompasses pyrrolidinyl, tetrahydrofuranyl, tetrahydrothienyl, tetrahydropyranyl, piperidinyl, piperazinyl, morpholinyl and thiomorpholinyl. Other heterocyclyl groups, for example 5-7 membered heterocyclyl, 5-6 membered heterocyclyl, 4 membered heterocyclyl, 5 membered heterocyclyl, 6 membered heterocyclyl and 7 membered heterocyclyl are as defined above but contain different numbers of ring atoms.

When n is 0, suitably R¹ and R² are both CH₃ or CH₂CH₃, in particular both CH₃. When n is 1, suitably R¹ and R² are both H.

Variable m is an integer of at least 1. In one embodiment, in particular when component B is a gel, m is 1-infinity, or more suitably 2-infinity. In one embodiment, m is 1-5,000,000, 2-5,000,000, 1-1,000,000, 2-1,000,000, 1-500,000, 2-500,000, 1-400,000, 2-400,000, 1-200,000, 2-200,000, 1-100,000, 2-100,000, 1-50,000, 2-50,000, 1-20,000, 2-20,000, 1-10,000, 2-10,000, 1-5,000, 2-5,000, 1-2,000, 2-2,000, 1-1,000, 2-1,000, 1-500, 2-500, 1-100, 2-100, 1-50, 2-50, 1-20, 2-20, 1-10, 2-10, 1-5 or 2-5.

In one embodiment, m is 2-500,000, 50-500,000, 100-500,000, 500-500,000, 1,000-500,000 or 5,000-500,000. In one embodiment, m is 1-200,000, 2-200,000, 50-200,000, 100-200,000, 500-200,000, 1,000-200,000 or 5,000-200,000. In one embodiment, m is 1-100,000, 2-100,000, 50-100,000, 100-100,000, 500-100,000, 1,000-100,000 or 5,000-100,000. In one embodiment, m is 1-50,000, 2-50,000, 50-50,000, 100-50,000, 500-50,000, 1,000-50,000 or 5,000-50,000. In one embodiment, m is 1-20,000, 2-20,000, 50-20,000, 100-20,000, 500-20,000, 1,000-20,000 or 5,000-20,000. In one embodiment, m is 1-5,000, 2-5,000, 50-5,000, 100-5,000, 500-5,000 or 1,000-5,000. In one embodiment, m is 1-20,000, 2-20,000, 50-20,000, 100-20,000, 500-20,000, 1,000-20,000 or 5,000-20,000. In one embodiment, m is 1-1,000, 2-1,000, 50-1,000, 100-1,000, or 500-1,000. In one embodiment, m is 1,000, 5,000, 20,000, 100,000, 200,000, or 500,000. In one embodiment, m is 11-44, 44-110 or 110-4200. In one embodiment, m is 1-10, 10-100, 100-6,200 or 1-infinity. In one embodiment, m is 1-1,000, e.g. 1-500, 1-100, 1-50, 1-25, 1-20 or 1-10.

In one embodiment, m is 2-100, e.g. 2-50, 2-25, 2-20 or 2-10. In one embodiment, m is 1, 2, 3 or 4; e.g. m is 2, 3 or 4, and in particular is 2 or 3. In another embodiment, m is 5-10 for example m is 5-8. In another embodiment, m is 5,000-100,000, 5,000-50,000 or 5,000-20,000.

Compounds of Formula (I) having m of at least 2 are particularly suitable as they can react with a component C having two or more amine groups to form a polymer, which is particularly suitable for use as a coating material.

In one embodiment, the weight average molecular weight of component B is 5,000 Da to 1,000,000 Da, such as 5,000 Da to 20,000 Da or 50,000 Da to 1,000,000 Da.

In one embodiment, component B is of Formula (Ia):

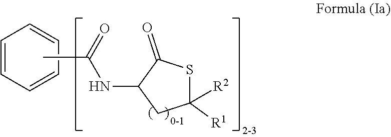

wherein,
R¹ and R² are independently selected from H and C₁₋₅ alkyl, e.g. selected from CH₃ and CH₂CH₃ (and in particular are both CH₃); or R¹ and R² together with the C atom to which they are attached combine to form a cycloheptyl or cyclohexyl ring which is optionally substituted on an available atom by one or more groups selected from C₁₋₂ alkyl and oxo.

In one embodiment, component B is:

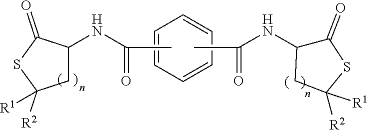

wherein n is 0 or 1, and R¹ and R² are independently selected from H and C₁₋₅ alkyl;
wherein, when n is 0, R¹ and R² are suitably both C₁₋₅ alkyl, in particular methyl or ethyl, e.g. are both methyl; and when n is 1, R¹ and R² are suitably both H.

In one embodiment, component B is:

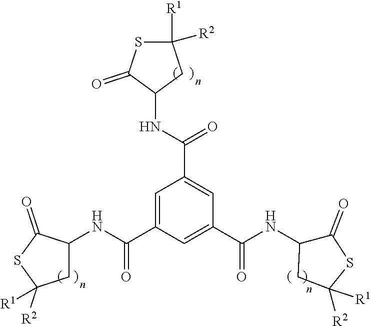

wherein n is 0 or 1, and R¹ and R² are independently selected from H and C₁₋₅ alkyl;
wherein, when n is 0, R¹ and R² are suitably both C₁₋₅ alkyl, in particular methyl or ethyl, e.g. are both methyl; and when n is 1, R¹ and R² are suitably both H.

In one embodiment, component B is:

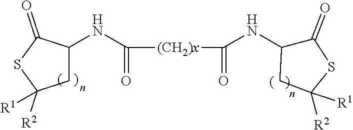

wherein n is 0 or 1, x is 1-10, for example 1-6, and R¹ and R² are independently selected from H and C₁₋₅ alkyl;
wherein, when n is 0, R¹ and R² are suitably both C₁₋₅ alkyl, in particular methyl or ethyl, e.g. are both methyl; and when n is 1, R¹ and R² are suitably both H.

In one embodiment, component B is of Formula (Iaa):

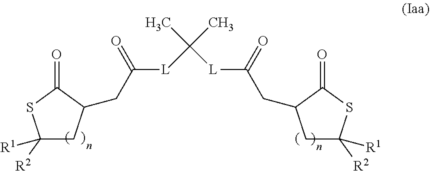

where L, n, R¹ and R² are as defined above.

More suitably, L is absent and component B is of Formula (lab):

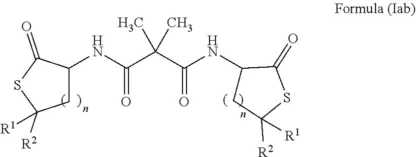

In a suitable embodiment, L is absent, n is 0 and component B is of Formula (lac):

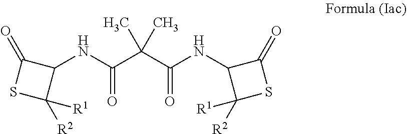

In compounds of Formulae (Iaa), (Iab) and (Iac), R¹ and R² are suitably independently selected from H and C₁₋₅ alkyl, suitably H, methyl or ethyl and more suitably H or methyl. In a suitable embodiment of compounds of Formula (Iac), and in compounds of Formulae (Iaa) and (Iab) in which n is 0, R¹ and R² are both methyl. In some compounds of Formulae (Iaa) and (Iab) in which n is 1, R¹ and R² are suitably both H.

In one embodiment, is provided a process for the preparation of a compound of Formula (I):

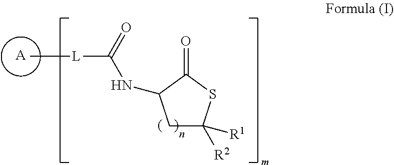

wherein,
A is a core moiety;
L is an optional linker;
R¹ and R² are independently selected from H and C₁₋₅ alkyl; or R¹ and R² together with the C atom to which they are attached combine to form a C₄₋₇ cycloalkyl ring or a 4-7 membered heterocyclic ring, both of which are optionally substituted on an available atom by one or more groups selected from C₁₋₂ alkyl and oxo;
n is 0 or 1;
m is an integer of at least 1;
the process comprising reacting a compound of Formula (IV) with a compound of formula (V) under suitable conditions to form a compound of Formula (I):

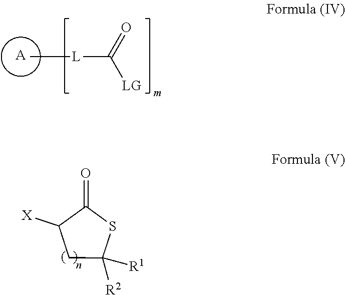

wherein,
for Formula (IV):
LG is a leaving group or an electrophilic group, or moiety C═O-LG is an electrophilic group;
A is a core moiety;
L is an optional linker; and
m is an integer of at least 1;
and for Formula (V):
n is 0 or 1;
R¹ and R² are independently selected from H and C₁₋₅ alkyl; or R¹ and R² together with the C atom to which they are attached combine to form a C₄₋₇ cycloalkyl ring or a 4-7 membered heterocyclic ring, both of which are optionally substituted on an available atom by one or more groups selected from C₁₋₂ alkyl and oxo; and
X is NH₂ or NH₃⁺Y⁻, wherein Y⁻ is a suitable anion e.g. Cl⁻;
LG can be leaving group such as halo (e.g. chloro or bromo), OMs or OTs. Alternatively LG can be an electrophilic group, e.g. a group that is capable of reacting with group X (NH₂ or NH₃*Y⁻) to forma covalent bond, such as azide, epoxide or isocycanate. Alternatively, moiety C═O-LG is an electrophilic group, for example the moiety C═O-LG comprises an ester (such as a succinimidyl ester), a carbonate (such as a succinimidyl carbonate) or an anhydride. In one embodiment, the reaction is carried out in the presence of aqueous base and/or buffer. In another embodiment, the reaction is carried out in organic solvent.

General Procedures for the synthesis of compounds of Formula (I) are provided in the Experimental section below, and specific example syntheses may be found in Examples 2-7. In the process above, a compound of Formula (I) includes compounds of Formulae (Ia), (Iaa), (Iab) and (Iac) as well as the other embodiments of component B shown above.

Starting materials of Formulae (IV) and (V) are either commercially available or may be prepared by known methods, for example, compounds of Formula (V) in which m is 1 and R¹ and R² are each methyl may be prepared as described by Martens et al, (1991).

It should be noted that component B contains at least one chiral centre. For example, in one embodiment comprising a single chiral centre (m is 1), the chiral centre is labelled * below:

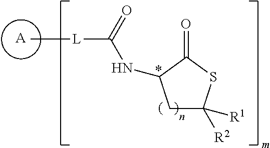

When m is greater than 1, component B will contain at least two chiral centres. In such embodiments, component B may be chiral or non-chiral (meso). In one embodiment, component B is a D-stereoisomer. In another embodiment, component B is an L-stereoisomer. In another embodiment, component B is a mixture (such as a racemic mixture) of D- and L-stereoisomers. Suitably, component B is a D-stereoisomer. It should be noted that for structures described herein where the stereochemistry of the chiral centre is undefined, said structure is intended to cover the D-stereoisomer, the L-stereoisomer, and all mixtures (including racemic mixtures) thereof.

### Component C

Component C is a moiety comprising one or more amine groups. The moiety may be a single compound or a mixture of compounds.

In the context of the present invention the term “amine group” refers to a primary, secondary or tertiary amine group, more usually a primary or secondary amine group. The terms “amine group” and “amino group” are used interchangeably.

In some embodiments, component C may be a single monomeric compound (i.e. a single compound of well defined composition).

In other embodiments, component C may be a polymer. As is known in the art, polymers are commonly mixtures of compounds formed as the product of a polymerisation reaction which may have proceeded to a different extent in different members in the mixture and can be defined in terms of their average molecular weight, e.g. their number average molecular weight, Mₙ and/or weight average molecular weight, M_{w} and/or z average molecular weight, M_{z} and/or polydispersity index, M_{w}/Mₙ.

In still other embodiments, component C may be a mixture of two or more compounds, especially two compounds, wherein the two or more compounds are selected from single monomeric compounds and polymers.

For example, component C may be a mixture of two or more monomeric compounds. Suitably, at least one or all of the two or more monomeric compounds may comprise two or more amine groups e.g. two amine groups. Such monomeric compounds are capable of polymerising with a component B in which n is 2 or more, e.g. 2.

Alternatively, component C may be a mixture of two or more compounds wherein the two or more compounds are two or more polymers.

In the context of component C, the term “polymer” refers to macromolecules comprising 2 or more monomer units up to e.g. 5,000,000 such as 11 or more monomer units, for example 11 to 5,000,000, 11-1,000,000, 11-500,000, 11-400,000, 11-200,000, 11-100,000, 11-50,000, 11-20,000, 11-10,000, 11-5,000, 11-2,000,11-1,000,11-500, 11-100, 11-50 or 11-20 monomer units.

A person of skill in the art will be aware that the length of polymer chains will vary from molecule to molecule of the polymer and that the numbers of monomer units given above therefore represent average numbers.

The polymer may be formed by polymerization of a selected number of monomers, which may be of one or more types. In some cases, the polymer is formed from a single monomer and examples of such polymers include polyethylene imine, polylysine, polypropylene oxide and polyethylene oxide. Alternatively, the polymer may be a copolymer formed from two or more monomers, for example two monomers. An example is a polymer formed from units of propylene oxide and ethylene oxide. Such copolymers may be block copolymers or random copolymers, for example block copolymers.

In some cases, the monomer units forming the polymer are alkylene oxide units or mixtures of alkylene oxide units, for example ethylene oxide or propylene oxide units or mixtures thereof. Alternatively, or in addition, the monomer units have pendant amine groups, for example the monomer units may be an alkylene imine, allylamine, lysine, arginine or an amino silane.

In some cases, the polymer may be a straight chain polymer. Alternatively, the polymer may be a branched or hyperbranched polymer or a dendrimer.

The amine groups of Component C are independently primary or secondary amine groups. In Component C, secondary amine groups may be present in a cyclic amine such as morpholine, piperidine or piperazine. Alternatively, secondary amine groups may be present as NH(C₁₋₆ alkyl) groups.

In one embodiment, component C comprises a single amine group, where the single amine group is a primary or secondary amine (where “single” in this context means that component C contains only one amine group). In this case, typically component C is a single monomeric compound or a mixture of two or more monomeric compounds. In one embodiment, component C is selected from the group consisting of aniline, benzylamine, morpholine, diethylamine, dibutylamine, 1-ethylpropylamine, 2-aminopentane, piperidine, 4-methylpiperidine, pyrrolidine, t-butylamine, t-amylamine, propylamine, butylamine, amylamine, isopentylamine, hexylamine, heptylamine, octylamine, nonylamine, decylamine, dodecylamine, hexadecylamine, octadecylamine, 4-methylaniline, 2-fluorobenzylamine, 3-fluorobenzylamine, 4-fluorobenzylamine, 3-(trifluoromethyl)benzylamine, 4-(trifluoromethyl)benzylamine and 3,5-bis(trifluoromethyl)benzylamine. In a suitable embodiment where C contains a single amine group, component C is selected from aniline, benzylamine and morpholine.

In another embodiment, component C comprises two or more amine groups, wherein said amine groups are independently primary amine groups or secondary amine groups. In another embodiment, component C comprises two or more primary amine groups. In another embodiment, component C comprises a mixture of primary amine groups and secondary amine groups.

In one embodiment, component C comprises a polyamine compound, e.g. polyethyleneimine, polyallylamine, polylysine, polyarginine or polyaminosilane. Thus, in one embodiment, component C comprises one or more moieties independently selected from the group consisting of polyethyleneimine, polyallylamine, polylysine, polyarginine and polyaminosilane. In a suitable embodiment, component C comprises polyethyleneimine (PEI), in particular branched polyethyleneimine. Suitably, the polyethyleneimine (in particular branched polyethyleneimine) has molecular weight of 10-1,000 kDa e.g. 10-50 kDa or 50-100 kDa. In one embodiment, component C is PEI.

In one embodiment, component C comprises a polyamine compound comprising a dendrimer or a hyperbranched polymer.

When component C is a straight chain polymer, it may comprise two amine groups, one at each terminus of the polymer chain. Suitably, the amine groups are primary amine groups or secondary amine groups. For example, both amine groups may be primary amine groups or both amine groups may be secondary amine groups or there may be a primary amine group at one terminus and a secondary amine group at the other terminus.

In one embodiment, component C comprises or consists of one or more, for example one or two polyalkylene oxide polymers, wherein each polymer has one or more terminal amino groups.

Suitably, component C comprises one or more straight chain polyalkylene oxide monomer units with an amino group at each terminus.

In one embodiment, component C comprises a single straight chain polyalkylene oxide polymer having an amino group, suitably a primary amino group or a secondary amino group at each terminus.

In another embodiment, component C comprises a mixture of two or more, suitably two, straight chain polyalkylene oxide polymers, wherein each polymer has an amino group, suitably a primary amino group or a secondary amino group, at each terminus.

In these embodiments the secondary amino group may be a group NH(C₁₋₆ alkyl), suitably NH(C₁₋₃ alkyl) such as NH(CH₂CH₃) or NH(CH₃).

In one embodiment, component C is a compound of Formula (X) or a mixture of two or more compounds of Formula (X): D G-E)

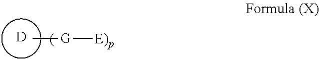

wherein, D is a core moiety, which in one embodiment, comprises one or more moieties independently selected from the group consisting of alkyl, spiroalkyl, aryl, heteroaryl, alkyl-aryl, a porphyrin, a polymer and a macrocycle, wherein alkyl, spiroalkyl, aryl, heteroaryl and alkyl-aryl are optionally substituted (e.g. by one or more substituents selected from the group consisting of alkyl, alkoxy, fluoroalkyl and fluoroalkoxy).

Suitably, D is a polymer comprising one or more C₂₋₄ alkylene oxide units, for example ethylene oxide units and/or propylene oxide units (i.e. D is a polyoxyC₂₋₄ alkylene). The core moiety D may comprise a single C₂₋₄ alkylene oxide unit but will more usually be a polymer comprising from 2 to 100 C₂₋₄ alkylene oxide units. In this case, the core moiety is suitably a straight chain polymer.

When the core moiety D is a polymer comprising two or more C₂₋₄ alkylene oxide units, the number of alkylene oxide units is expressed as an average since the length of the polymer chains will vary from molecule to molecule of the polymer.

In some cases, component C is a single compound of Formula (X) wherein the average number of alkylene oxide units in the core moiety D may be from about 2 to about 20, for example about 2 to 10, 3 to 7 or 3.5 to 6.

Alternatively, component C may be a mixture of a first compound of Formula (X) and a second compound of Formula (X). For example, a first compound of Formula (X) wherein D is a polymer which comprises an average number of alkylene oxide units from about 2 to about 20, for example about 2 to 10, 3 to 7 or 3.5 to 6, and a second compound of Formula (X) wherein D is a polymer which comprises an average number of alkylene oxide units from about 11 to about 100, for example about 20 to about 60 or 30 to 50.

In some compounds of Formula (X), the alkylene oxide units will all be the same, for example, all the alkylene oxide units will be ethylene oxide units or all the alkylene oxide units will be propylene oxide units.

Other compounds of Formula (X) comprise two or more different types of alkylene oxide unit. For example in a compound of Formula (X), the core moiety D may be a polymer which comprises both ethylene oxide units and propylene oxide units. These may be present in a fixed ratio on average, although the ratio may vary between individual molecules of the oligomer or polymer. The order in which the ethylene oxide units and propylene oxide units are assembled in a polymeric structure is random and may vary between molecules.

In compounds of Formula (X), where D is a moiety such as a polymer comprising one or more C₂₋₄ alkylene oxide units, the linker G is suitably absent and/or p is suitably 2.

As a further alternative, D may be a polyalkylsiloxane moiety substituted with one or more primary and/or secondary amine groups, especially one or more primary amine groups.

Again, in polyalkylsiloxane compounds of Formula (X), the linker G is suitably absent and/or p is suitably 2.

When core moiety D comprises an alkyl group (e.g. C₁₋₂₀ alkyl, C₁₋₁₀ alkyl, C₁₋₆ alkyl, C₁₋₄ alkyl or C₁₋₃ alkyl (e.g. methyl or ethyl)), it is optionally substituted by one or more substituents (e.g. selected from the group consisting of alkyl, alkoxy, fluoroalkyl and fluoroalkoxy). The alkyl group can be branched or unbranched. In one embodiment, core moiety D is an alkyl group.

When core moiety D comprises a spiroalkyl group (e.g. C₁₋₂₀ spiroalkyl, C₁₋₁₀ spiroalkyl or C₁₋₆ spiroalkyl) it is optionally substituted by one or more substituents (e.g. selected from the group consisting of alkyl, alkoxy, fluoroalkyl and fluoroalkoxy). In one embodiment, core moiety D is a spiroalkyl group.

When core moiety D comprises an aryl group (e.g. 5-20 membered aryl, 5-12 membered aryl, 5-membered aryl or 5-7 membered aryl) it is optionally substituted by one or more substituents (e.g. selected from the group consisting of alkyl, alkoxy, fluoroalkyl and fluoroalkoxy). “Aryl” as used herein is a cyclic group with aromatic character, such as phenyl or naphthyl. Aryl also includes diaryl and polyaryl groups. In a suitable embodiment, aryl is phenyl, which is optionally substituted by one or more substituents selected from the group consisting of alkyl, alkoxy, fluoroalkyl and fluoroalkoxy. In one embodiment, core moiety D is an aryl group.

When core moiety D comprises an heteroaryl group, it is optionally substituted by one or more substituents (e.g. selected from the group consisting of alkyl, alkoxy, fluoroalkyl and fluoroalkoxy). In one embodiment, heteroaryl is 5-10 membered heteroaryl, e.g. pyrrolyl, furanyl, thienyl, imidazolyl, pyrazolyl, thiazolyl, isothiazolyl, oxadiazolyl, thiadiazolyl, triazolyl, oxazolyl, isoxazolyl, tetrazolyl, pyridinyl, pyrimidinyl, pyradizinyl or pyrazinyl. In one embodiment, core moiety D is an heteroaryl group.

When core moiety D comprises an alkyl-aryl group (e.g. aryl substituted by alkyl (e.g. C₁₋₁₀ alkyl) or polyaryl linked by alkyl (e.g. C₁₋₅ alkyl)), it is optionally substituted by one or more substituents (e.g. selected from the group consisting of alkyl, alkoxy, fluoroalkyl and fluoroalkoxy). In one embodiment, core moiety D is an alkyl-aryl group.

In one embodiment, core moiety D comprises a porphyrin e.g. tetra-4-aryl-meso. In one embodiment, core moiety D is a porphyrin.

In one embodiment, core moiety D comprises a polymer (e.g. a polyamine compound, a dendrimer or a hyperbranched polymer). In one embodiment, the polyamine compound is selected from the group consisting of polyethyleneimine, polyallylamine, polylysine, polyarginine and polyaminosilane. In one embodiment, core moiety D comprises polyethyleneimine (PEI), in particular branched polyethyleneimine. Suitably, the polyethyleneimine (in particular branched polyethyleneimine) has molecular weight of 10-1,000 kDa e.g. about 10-50 kDa or 50-100 kDa. In one embodiment, core moiety D comprises a dendrimer, e.g. a polyamine dendrimer such as a PAMAM dendrimer or a PPI-dendrimer. In one embodiment, core moiety D is a polymer.

In one embodiment, core moiety D comprises a macrocycle, e.g. a cyclodextrin. In one embodiment, core moiety D is a macrocycle.

In one embodiment, core moiety D comprises secondary amine groups. In one embodiment, core moiety D comprises C₁₋₁₀ alkylene, aryl, aryl-(CH₂)₁₋₁₀-aryl or —[CH₂CH₂NH]_{1-20,000}—.

G is an optional linker. In one embodiment, G comprises the moiety —(CH₂CH₂NH)₁₋₅—CH₂CH₂—. In another embodiment, G is absent.

E is N(R^{z})H, where each R^{z} is H or C₁₋₆alkyl. More suitably, each R^{z} is independently H or C₁₋₃ alkyl, such as H, methyl or ethyl. In one embodiment, each E is NH₂. In a further embodiment, each E is NH(C₁₋₆ alkyl), for example NH(CH₃). In a further embodiment, p>1 and one or more E is NH₂ and one or more E is NH(C₁₋₆ alkyl). For example, when p is 2, both E groups may be NH₂ groups or both E groups may be NH(C₁₋₆ alkyl), such as NH(CH₃) or one E group may be NH₂ and the other E group may be NH(C₁₋₆ alkyl), such as NH(CH₃).

Variable p is an integer of at least 1. In one embodiment, in particular when component C is a gel, p is 1-infinity, or more suitably 2-infinity. In one embodiment, p is 1-5,000,000, 2-5,000,000, 1-1,000,000, 2-1,000,000, 1-500,000, 2-500,000, 1-400,000, 2-400,000, 1-200,000, 2-200,000, 1-100,000, 2-100,000, 1-50,000, 2-50,000, 1-20,000, 2-20,000, 1-10,000, 2-10,000, 1-5,000, 2-5,000, 1-2,000, 2-2,000, 1-1,000, 2-1,000, 1-500, 2-500, 1-100, 2-100, 1-50, 2-50, 1-20, 2-20, 1-10, 2-10, 1-5 or 2-5.

In one embodiment, p is 2-500,000, 50-500,000, 100-500,000, 500-500,000, 1,000-500,000 or 5,000-500,000. In one embodiment, p is 1-200,000, 2-200,000, 50-200,000, 100-200,000, 500-200,000, 1,000-200,000 or 5,000-200,000. In one embodiment, p is 1-100,000, 2-100,000, 50-100,000, 100-100,000, 500-100,000, 1,000-100,000 or 5,000-100,000. In one embodiment, p is 1-50,000, 2-50,000, 50-50,000, 100-50,000, 500-50,000, 1,000-50,000 or 5,000-50,000. In one embodiment, p is 1-20,000, 2-20,000, 50-20,000, 100-20,000, 500-20,000, 1,000-20,000 or 5,000-20,000. In one embodiment, p is 1-5,000, 2-5,000, 50-5,000, 100-5,000, 500-5,000 or 1,000-5,000. In one embodiment, p is 1-20,000, 2-20,000, 50-20,000, 100-20,000, 500-20,000, 1,000-20,000 or 5,000-20,000. In one embodiment, p is 1-1,000, 2-1,000, 50-1,000, 100-1,000, or 500-1,000. In one embodiment, p is 1,000, 5,000, 20,000, 100,000, 200,000, or 500,000. In one embodiment, p is 11-44, 44-110 or 110-4200. In one embodiment, p is 1-10, 10-100, 100-6,200 or 1-infinity. In one embodiment, p is 1-1,000, e.g. 1-500, 1-100, 1-50, 1-25, 1-20 or 1-10. In one embodiment, p is 2-100, e.g. 2-50, 2-25, 2-20 or 2-10. In one embodiment, p is 1, 2, 3 or 4; e.g. p is 2, 3 or 4, and in particular is 2 or 3. In another embodiment, p is 5-10 for example p is 5-8. In another embodiment, p is 5,000-100,000, 5,000-50,000 or 5,000-20,000. In one embodiment, p is p is 1-100,000, 2-100,000, 2-50,000, 2-20, 2-12, 2-5, 2-4 or 2-3.

In more suitable compounds of Formula (X), p is at least 2 since this enables formation of a polymer when the compound of Formula (X) reacts with a compound of Formula (I) in which m is at least 2. Most suitably p is 2.

In some cases, component C is a single compound of Formula (X). In other cases, component C is a mixture of two or more compounds of Formula (X), for example a mixture of two compounds of Formula (X).

In one embodiment, component C is p-phenylenediamine.

In one embodiment, component C is of Formula (Xx):

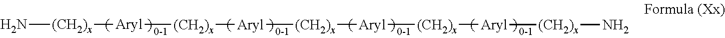

wherein each x is independently 0-10.

In one embodiment, component C is of Formula (Xxx):

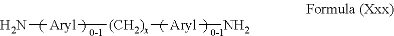

wherein x is 0-10.

In one embodiment, component C is of Formula (Xxy):

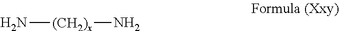

wherein x is 1-10, e.g. 3-8.

In one embodiment, component C is H₂N—(CH₂)₆—NH₂.

In one embodiment, component C is H₂N—(CH₂)₄—NH₂.

In one embodiment, component C is of Formula (Xxz):

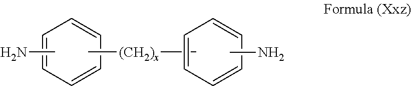

wherein x is 0-10, such as 1.

In a further embodiment, component C comprises a compound of Formula (Xu):

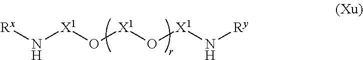

wherein X¹ is a straight or branched C₂₋₄ alkylene group, r is a number representing the average number of alkylene oxide units per molecule and each of R^{x} and R^{y} is H or C₁₋₆ alkyl, such as H or C₁₋₃ alkyl, for example H, methyl or ethyl, especially H or methyl. In some suitable embodiments R^{x} and R^{y} are both H. In other suitable embodiments R^{x} and R^{y} are both methyl. In still other suitable embodiments, one of R^{x} and R^{y} is H and the other of R^{x} and R^{y} is methyl.

The weight average molecular weight of compounds of Formula (Xu) is typically from about 100 g/mol to 10,000 g/mol, for example 100 g/mol to 5,000 g/mol 100 g/mol to 1,000 g/mol, 200 to 800 g/mol or 400 to 500 g/mol.

Examples of compounds of Formula (Xu) include a compound of Formula (Xva):

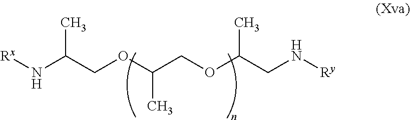

which is based on a core, D, comprising propylene oxide units; and a compound of Formula (Xvb)

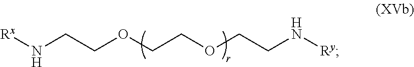

which is based on a core, D, comprising ethylene oxide units.

In Formulae (Xva) and (Xvb), R^{x} and R^{y} are as defined above for Formula (Xu).

In some cases, in the compound of Formula (Xu), r is from 2 to 10.

Suitably, the compound of Formula (Xu) is a compound of Formula (Xva) or Formula (Xvb) wherein r is typically a value of from about 2 to about 10, for example about 3 to 7 or 3.5 to 6.

Alternatively, in the compound of Formula (Xu) not all X¹ groups are the same, for example some X¹ groups may be ethylene oxide units and other X¹ groups may be propylene oxide units. In this case r is suitably from about 11 to about 100, for example about 20 to about 60 or 30 to 50.

In some cases, when the compound of Formula (Xu) is a polymer comprising ethylene oxide units and propylene oxide units, it may be a polymer of formula (Xw):

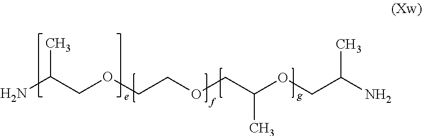

wherein e, f and g are each independently numbers representing the average number of alkylene oxide units per polymer molecule and wherein
e and g are each independently from 0 to 10 and the sum of e and g is from 2 to 15; and f is from about 2 to about 100;
provided that e and f are not both 0.

Suitably, e and g are each independently from 1 to 8, more suitably 2 to 5, the sum of e and g is from 2 to 10, more suitably from about 4 to 8 and f is from about 10 to 70, more suitably 20 to 60 and most suitably 30 to 50.

The weight average molecular weight of compounds of Formula (Xw) and similar polymers is typically from about 100 g/mol to 10,000 g/mol, for example 100 g/mol to 5,000 g/mol, 100 g/mol to 1,000 g/mol or 200 to 800 g/mol, for example about 600 g/mol.

In a further embodiment, component C comprises a mixture of two or more compounds of Formula (Xu), for example a mixture of two compounds of formula (Xu).

Suitably component C comprises a mixture of a first compound of Formula (Xu) and a second compound of Formula (Xu).

The first compound of Formula (Xu) may be a compound of Formula (Xva) or Formula (Xvb) as shown above wherein r is from about 2 to about 10, for example about 3 to 7 or 3.5 to 6. More suitably, the first compound of Formula (Xu) is a compound of Formula (Xva).

In the second compound of Formula (Xu), r is suitably from about 11 to about 100, for example about 20 to about 60 or 30 to 50. In this compound, not all X¹ groups must be the same, for example some X¹ groups may be ethylene oxide units and other X¹ groups may be propylene oxide units.

In a further embodiment, component C is an amine-functionalized polyalkylsiloxane of formula (Xt):

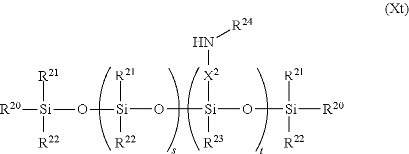

wherein each of R²⁰, R²¹, R²² and R²³ is independently C₁₋₆ alkyl, more suitably C₁₋₄ alkyl, R²⁴ is H or C₁₋₄ alkyl, especially H, X² is a straight or branched C₁₋₆ alkylene group, more suitably a straight or branched C₁₋₄ alkylene group, especially (CH₂)₂₋₄ and each of s and t is independently an integer of from about 10 to 400, more suitably from about 50 to 250. Typically, the molecular weight of the amine-functionalized polalkylsiloxane of formula (Xt) is from around 5,000 g/mol to about 100,000 g/mol, for example about 20,000 g/mol to 80,000 g/mol or about 30,000 to 70,000 g/mol, and typically about 50,000 g/mol.

An example compound of Formula (Xt) is an amine-functionalized polydimethylsiloxane (PDMS), in which each of R²⁰, R²¹, R²² and R²³ is methyl, R²⁴ is H and X² is (CH₂)₂₋₄, especially (CH₂)₃.

In an embodiment, the weight average molecular weight of this example compound is about 50,000 g/mol.

Formulae (Xw) and (Xt) are provided to illustrate the composition of the polymers, it being understood that while e, f and g in Formula (Xw) and s and t in Formula (Xt) are each independently numbers representing the average number of monomer units per polymer molecule, the individual monomer units may appear in such a polymer without restriction as to order.

### Reaction of Components B and C

When reacted together, components B and C form a chemical entity which may be a compound, a copolymer or a polymer depending on the nature of components B and C.

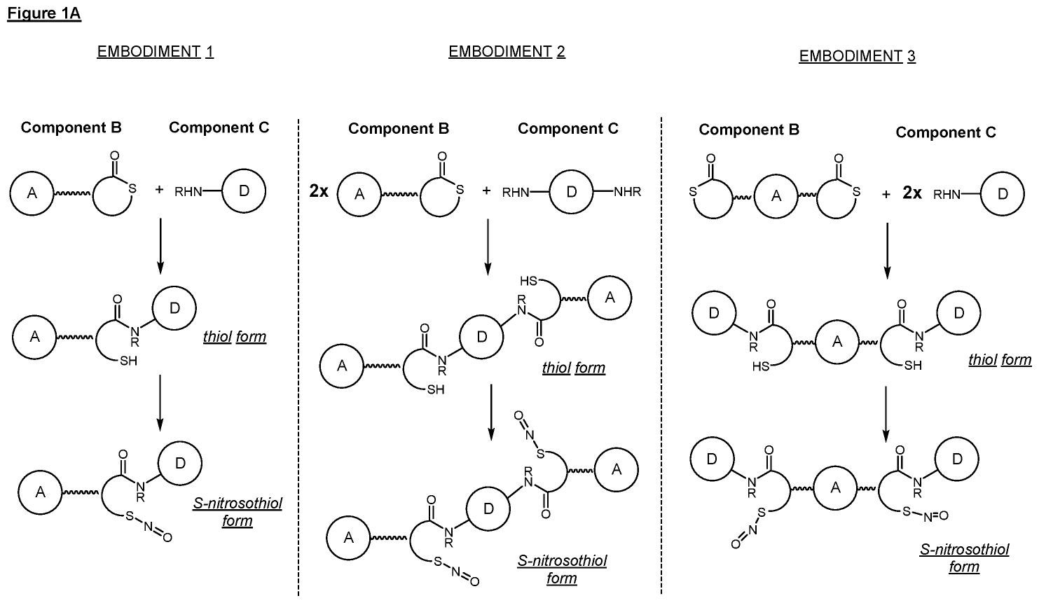

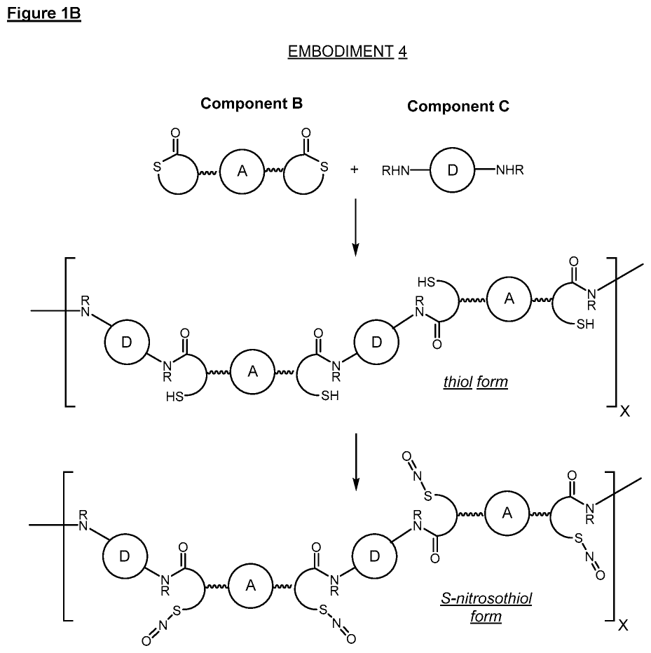

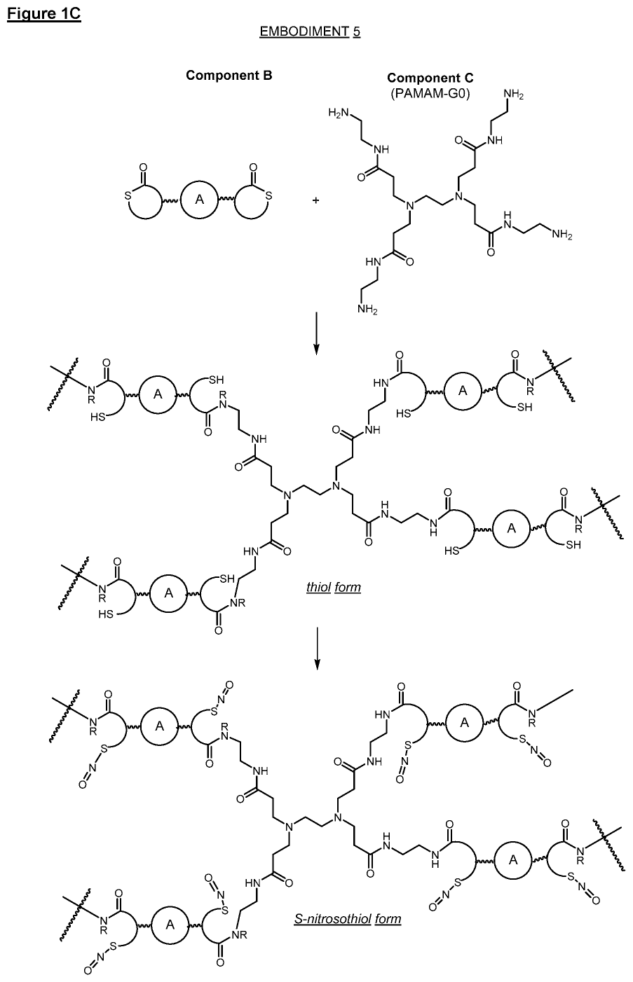

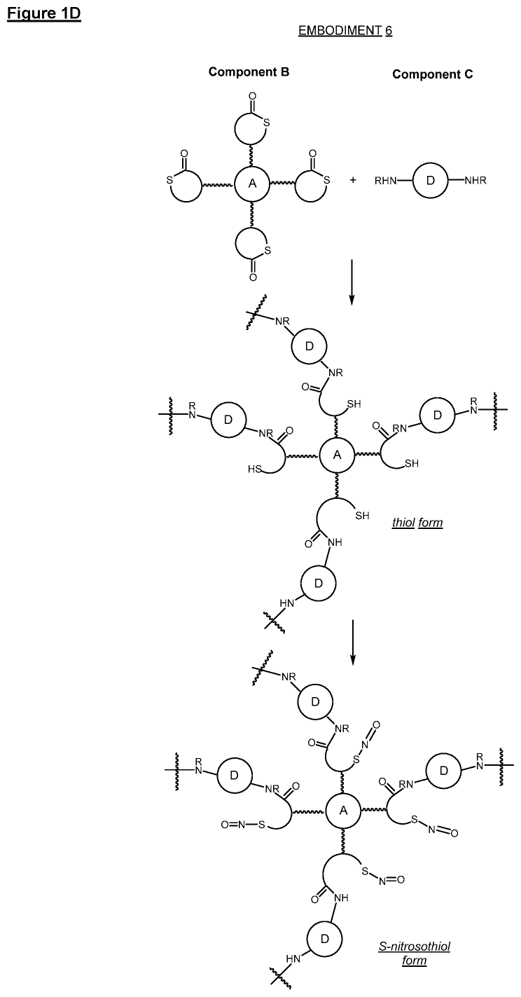

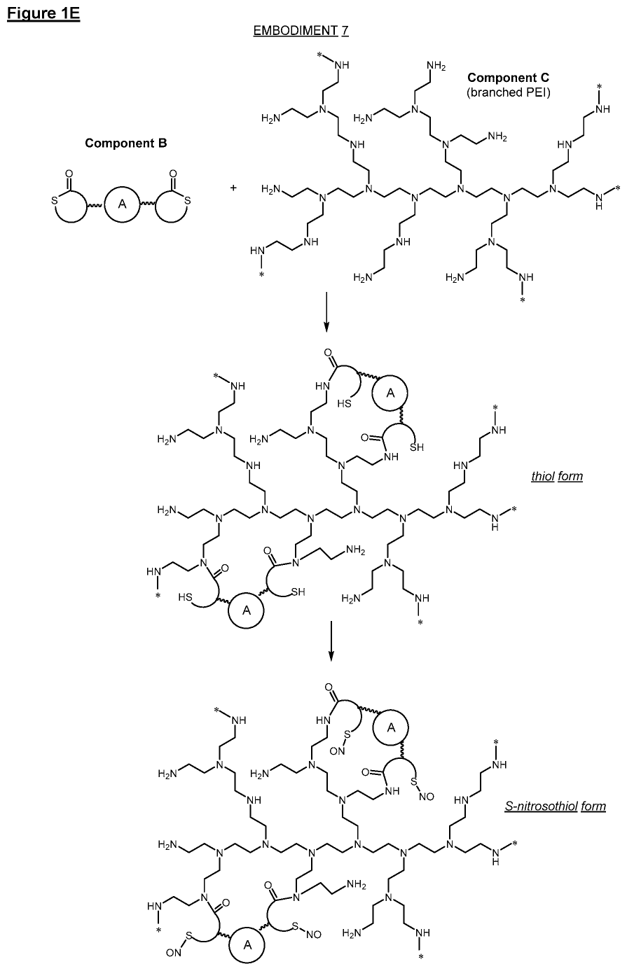

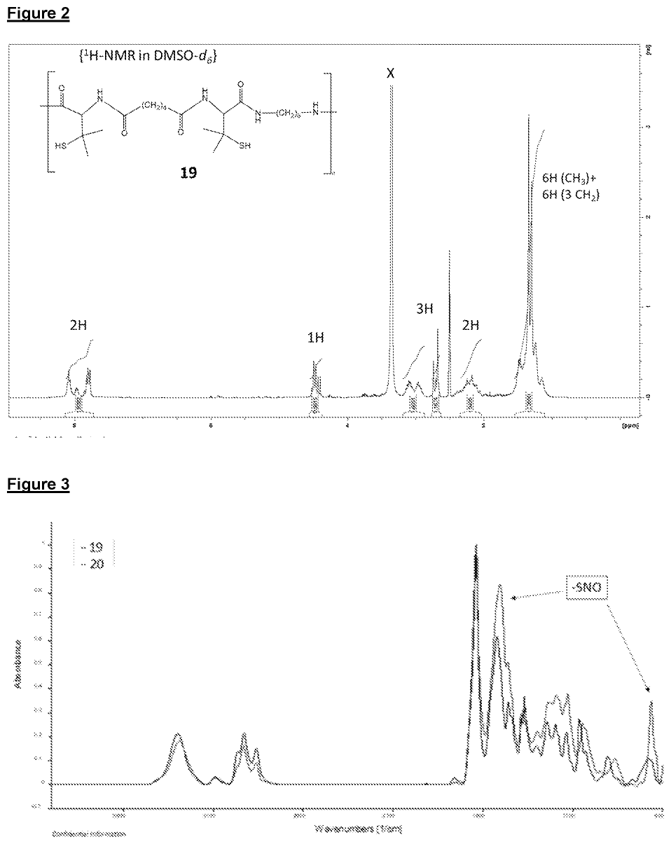

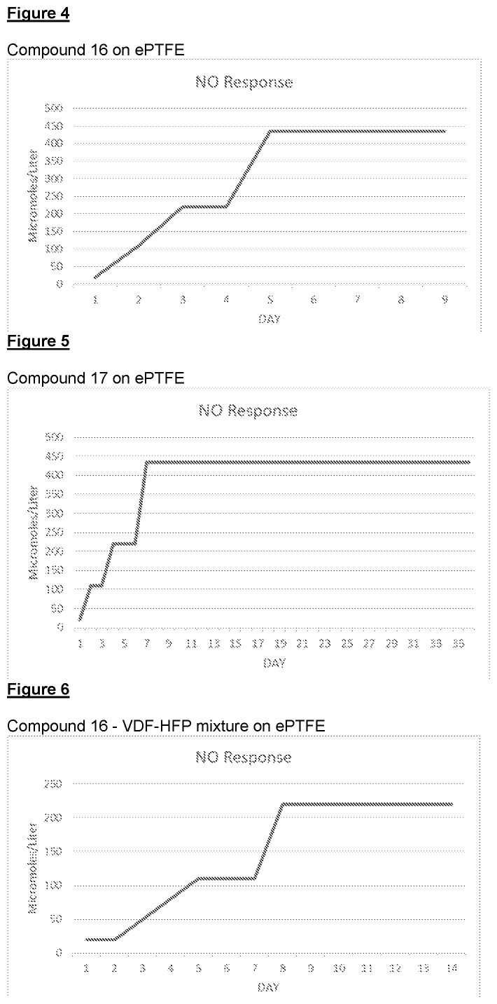

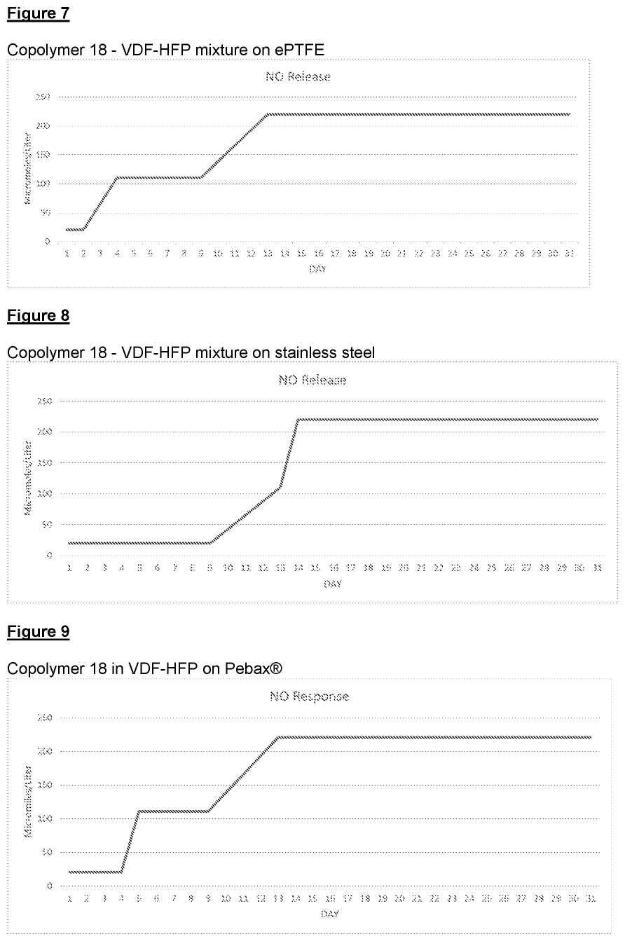

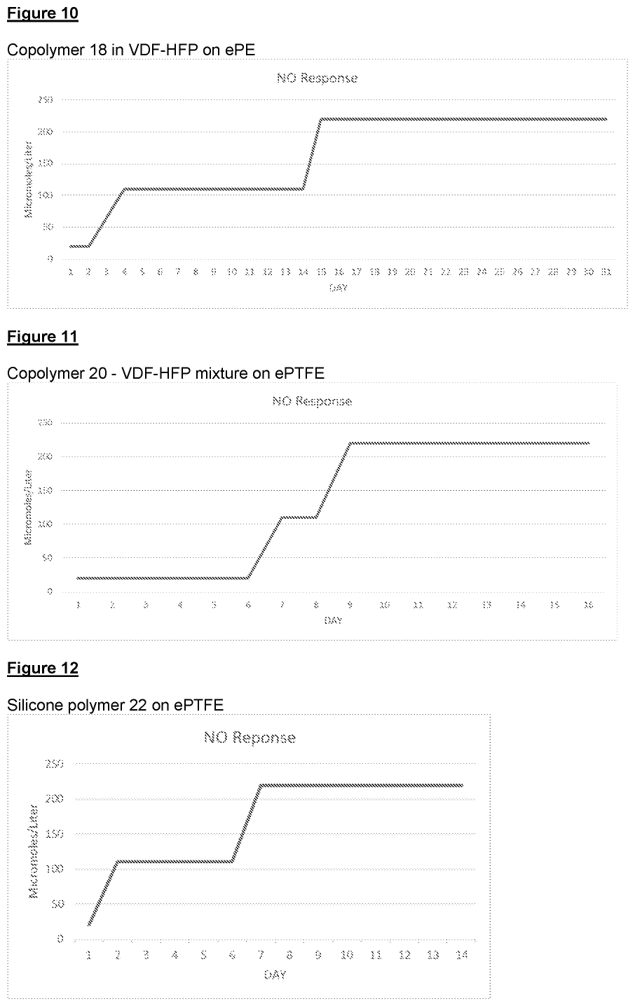

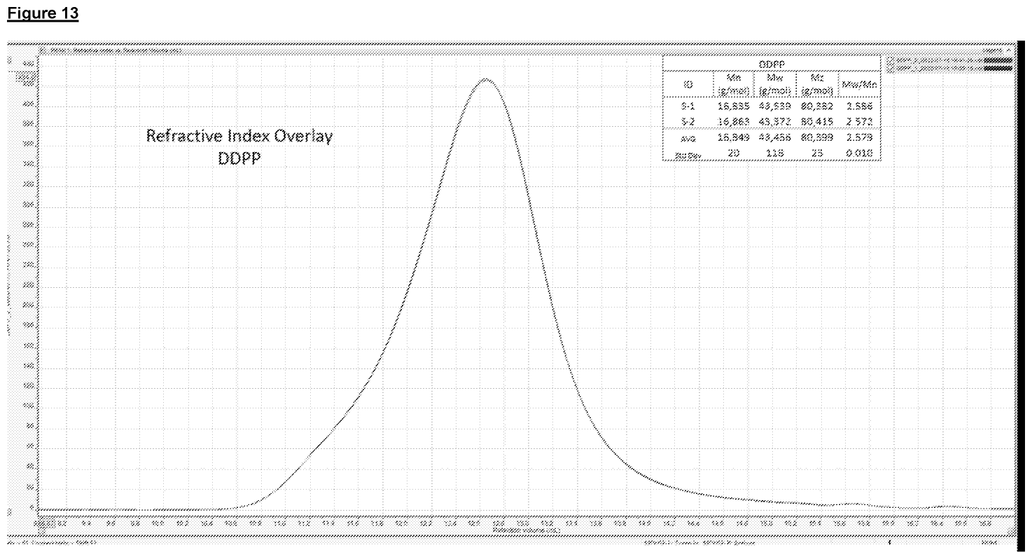

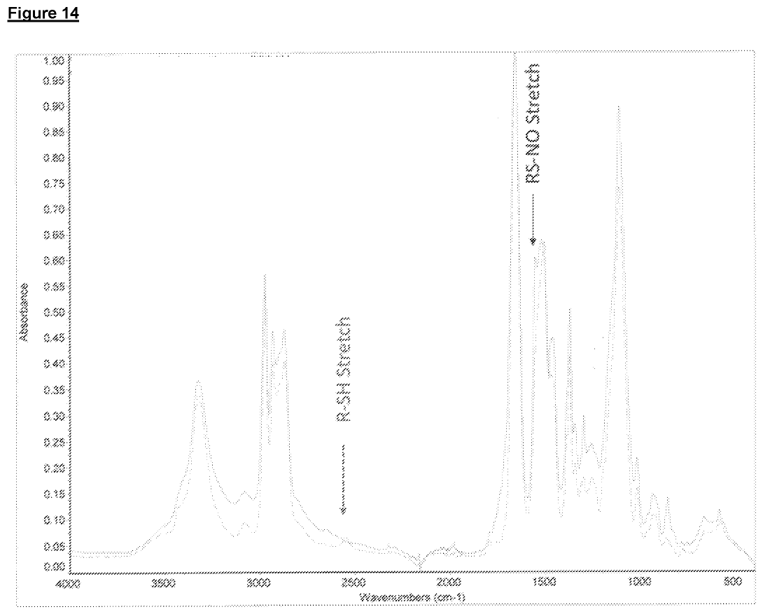

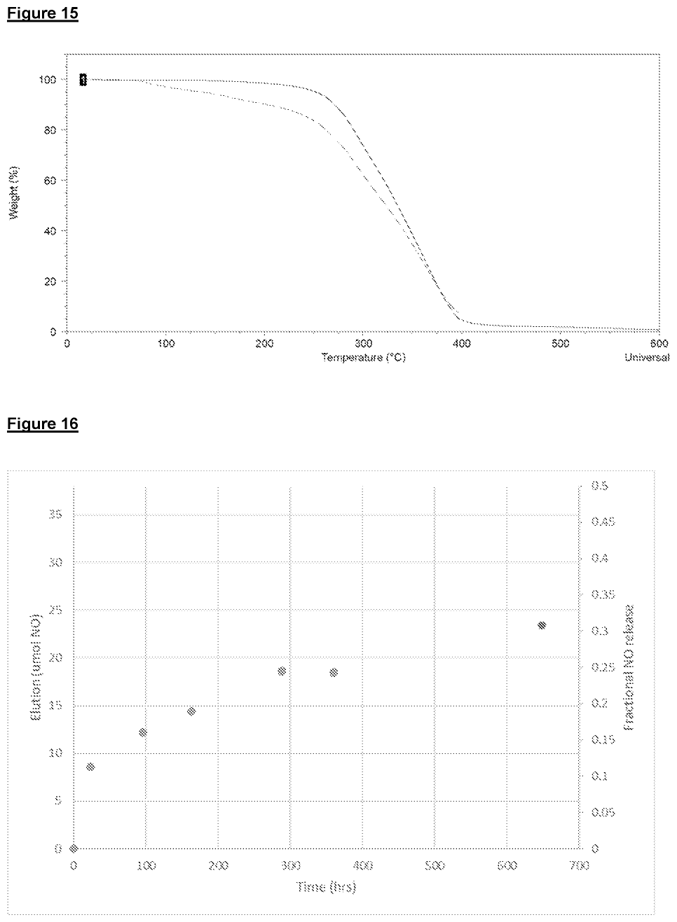

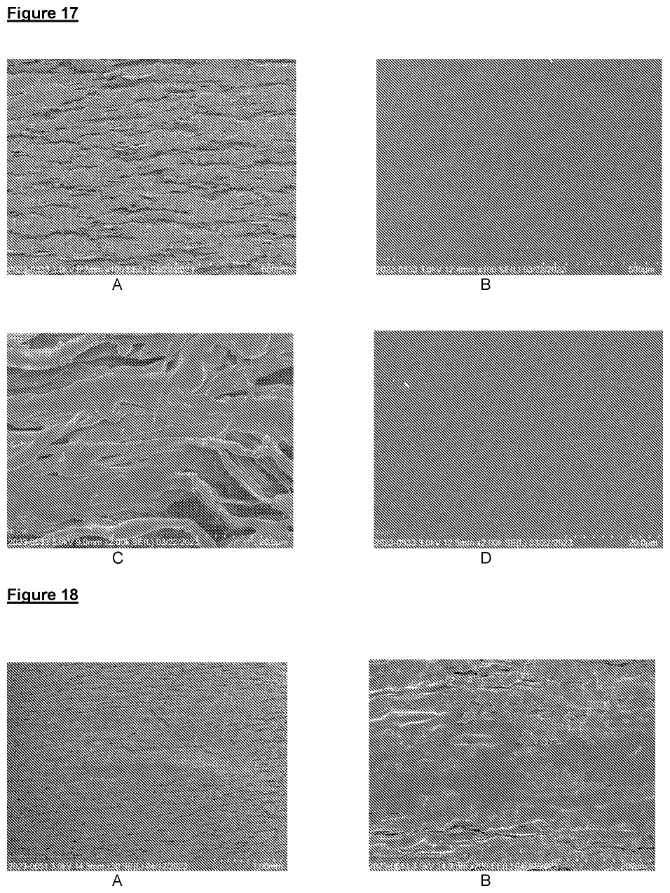

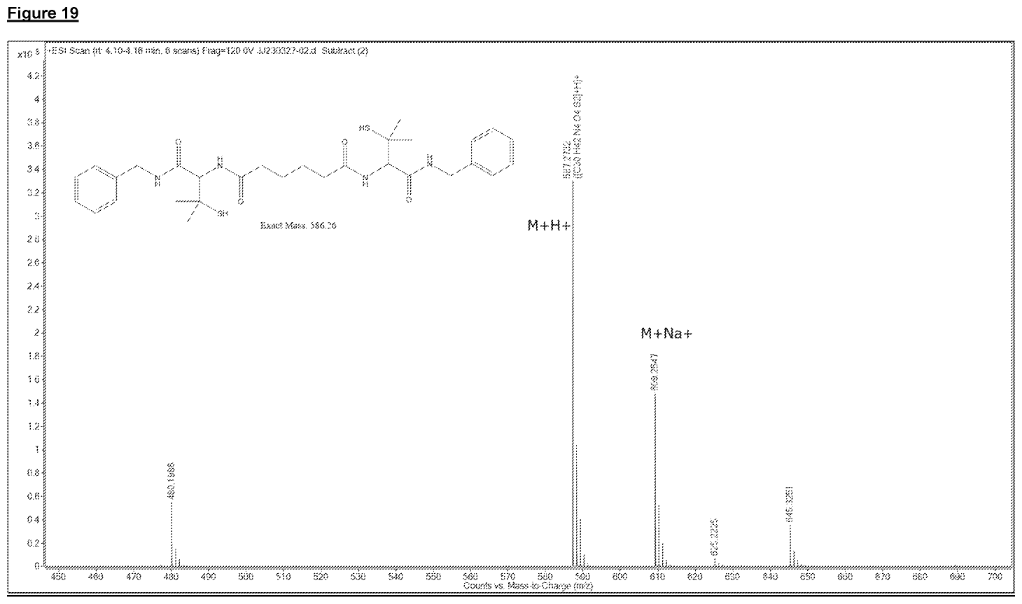

FIG. 1A (Embodiment 1) shows the reaction between a component B with a single thioester moiety and a component C with a single amine moiety. The resulting chemical entity (compound) has one thiol group (thiol form/—SH form). When exposed to suitable conditions (as described herein), the thiol can be converted to an S-nitrosothiol group (S-nitrosothiol/—SNO form).

FIG. 1A (Embodiment 2) shows the reaction between a component B with a single thioester moiety and a component C with two amine moieties. The resulting chemical entity (compound) has two thiol group (thiol form/—SH form). Again, the thiol groups can be converted to S-nitrosothiol groups (S-nitrosothiol/—SNO form).

FIG. 1A (Embodiment 3) shows the reaction between a component B with two thioester moieties and a component C with one amine moiety. The resulting chemical entity (compound) has two thiol group (thiol form/—SH form). Again, the thiol groups can be converted to S-nitrosothiol groups (S-nitrosothiol/—SNO form).

FIG. 1B (Embodiment 4) shows the reaction between a component B with two thioester moieties and a component C with two amine moieties. In this embodiment, reaction of components B and C leads to a straight chain polymer. The resulting chemical entity (copolymer) has multiple thiol groups (thiol form/—SH form). Again, some/all of the thiol groups can be converted to S-nitrosothiol groups (S-nitrosothiol/—SNO form). It should be noted that although the Figure shows all of the —SH groups being converted to —SNO groups, the embodiment also covers copolymers where only a proportion of the —SH groups have been converted to —SNO groups. In a sub-embodiment of Embodiment 4, component C is a mixture and more than one D moiety is present. For example, there are two types of D, say D1 and D2, and the product of reacting components B and C leads to a straight chain polymer in which moieties D1 and D2 are distributed randomly in the polymer chain.

FIG. 1C (Embodiment 5) shows the reaction between a component B with two thioester moieties and a component C which is a dendrimer, specifically PAMAM-GO. The resulting chemical entity (copolymer) has multiple thiol groups (thiol form/—SH form). Again, some/all of the thiol groups can be converted to S-nitrosothiol groups (S-nitrosothiol/—SNO form). It should be noted that although the Figure shows all of the —SH groups being converted to —SNO groups, the embodiment also covers copolymers where only a proportion of the —SH groups have been converted to —SNO groups.

FIG. 1D (Embodiment 6) shows the reaction between a component B with four thioester moieties and a component C with two amine moieties. The resulting chemical entity (copolymer) has multiple thiol groups (thiol form/—SH form). Again, some/all of the thiol groups can be converted to S-nitrosothiol groups (S-nitrosothiol/—SNO form). It should be noted that although the Figure shows all of the —SH groups being converted to —SNO groups, the embodiment also covers copolymers where only a proportion of the —SH groups have been converted to —SNO groups.

FIG. 1E (Embodiment 7) shows the reaction between a component B with two thioester moieties and component C which is PEI (polyethyleneimine). The resulting chemical entity (cross-linked polymer) has multiple thiol groups (thiol form/—SH form). Again, some/all of the thiol groups can be converted to S-nitrosothiol groups (S-nitrosothiol/—SNO form). Although the Figure shows all of the —SH groups being converted to —SNO groups, the embodiment also covers cross-linked polymers where only a proportion of the —SH groups have been converted to —SNO groups. As shown in the Figure, cross-linking can occur between primary and secondary amines, as well as between primary amines, and between secondary amines. It should be noted that the Figure is merely representative in the sense that more or fewer cross-linkages can be formed, depending on the amount of component B that is reacted with component C.

The embodiments of FIGS. 1B, 1C, 1D and 1E are advantageous because the reaction between component B and component C results in the production of a polymeric chemical entity which is particularly suitable for use as a coating material.

Thus, when m in the compound of Formula (I) (i.e. component B) is 2 or more, for example 2, the chemical entity produced by the reaction with component C is a polymer.

In cases when m of component B is 2 and component C comprises a single compound (either a single monomeric compound or a polymer), for example a compound of Formula (X), the chemical entity produced by the reaction between components B and C will be a copolymer having alternating units derived from components B and C. The synthesis of chemical entities of this type is shown below in Examples 17, 18, A2 and A3.

In other cases, m of component B is 2 and component C comprises a mixture of a first component C compound and a second component C compound. The first and second component C compounds may be either single monomeric compounds or polymers and may, for example be different compounds of Formula (X). In these cases, the chemical entity produced by the reaction between components B and C will be a copolymer comprising units derived from component B alternating with units derived from either the first component C compound or the second component C compound. The order in which the first and second component C compounds are incorporated into any given molecule of the copolymer will be random. The synthesis of a chemical entity of this type is shown below in Example A4

The polymers of the embodiments of FIGS. 1B, 1C, 1D and 1E suitably have weight average molecular weights of from about 500 g/mol to 2,000,000 g/mol, for example from about 10,000 g/mol to 1,000,000 g/mol, 20,000 g/mol to 700,000 g/mol, 30,000 g/mol to 500,000 g/mol or 35,000 g/mol to 200,000 g/mol.

In one embodiment, the chemical entity comprises;

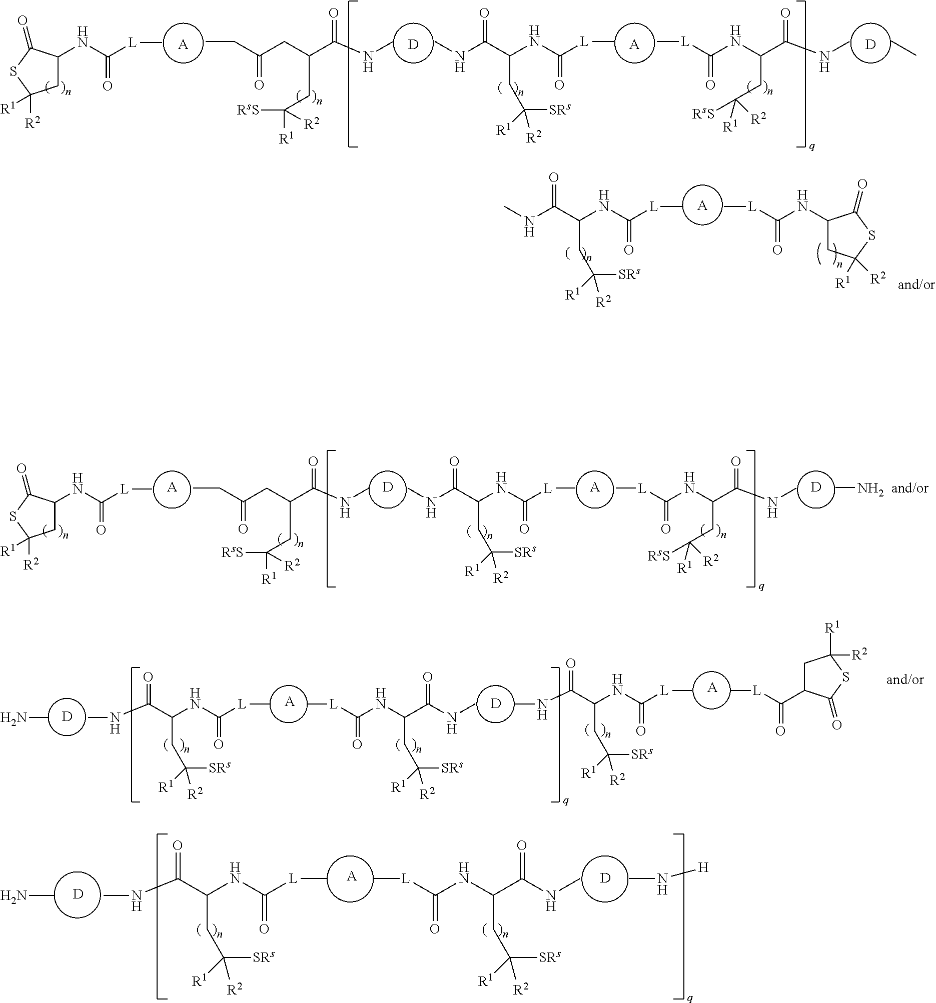

wherein,
A, L, n, R¹, R², and 0 are as defined herein;
R^{s} is H or N═O; and
q is 10 to 1000, for example 15 to 500, 20 to 250 or 25 to 180.

In one embodiment, the chemical entity comprises:

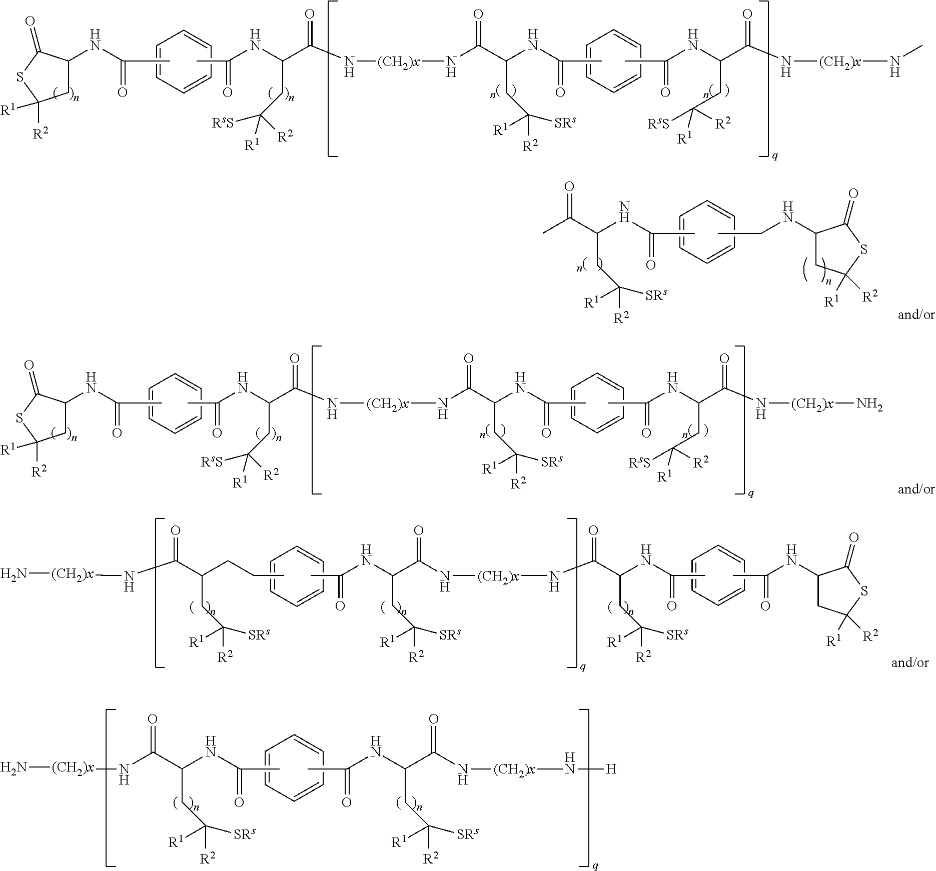

wherein n is as defined herein;
x is 1-10, for example 2-6;
R^{s} is H or N═O; and
q is 10 to 1000, for example 15 to 500, 20 to 250 or 25 to 180.

Typically, the weight average molecular weight (M_{w}) of a polymer of this type is from about 10,000 g/mol to 100,000 g/mol, for example 20,000 g/mol to 80,000 g/mol or 30,000 g/mol to 60,000 g/mol.

In a further embodiment, the chemical entity comprises:

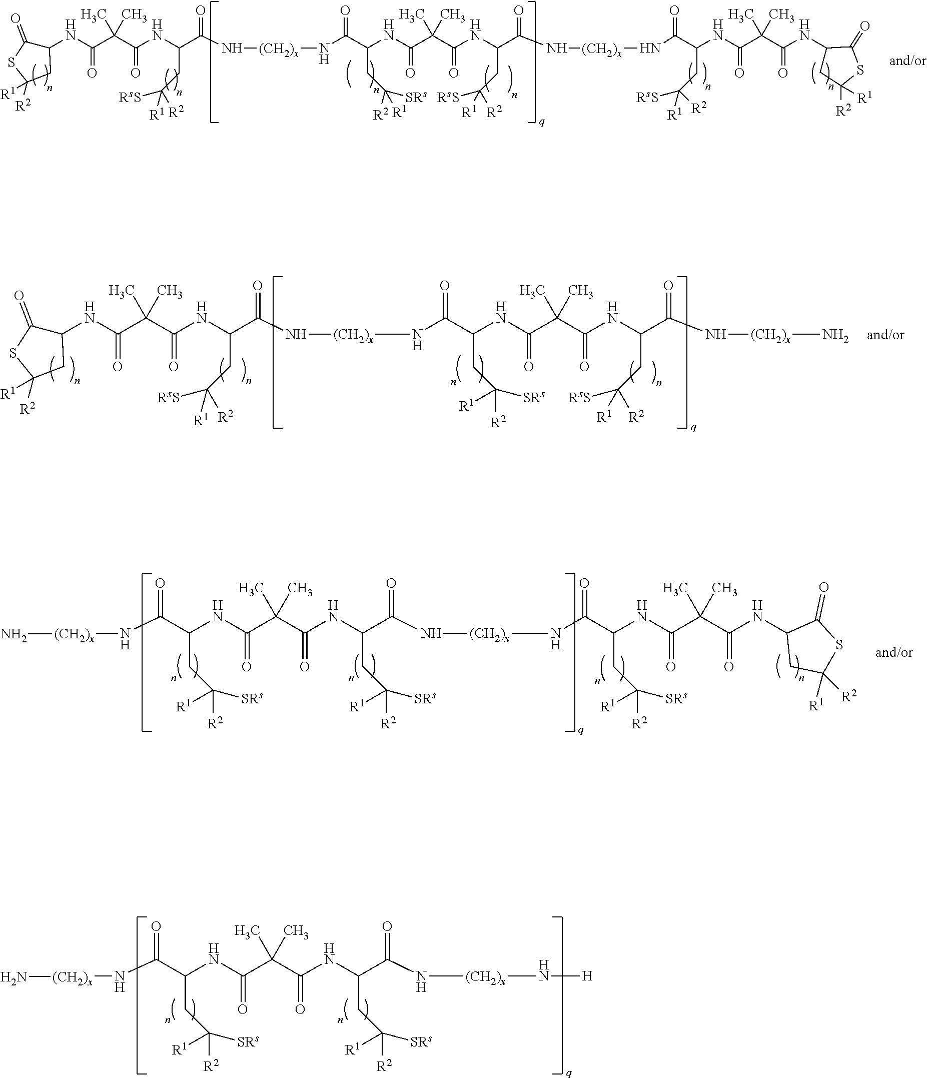

x is 1-10, for example 2-6;
R^{s} is H or N═O; and
q is 10 to 1000, for example 15 to 500, 20 to 250 or 25 to 180.

Typically, the weight average molecular weight (M_{w}) of a polymer of this type is from about 10,000 g/mol to 100,000 g/mol, for example 20,000 g/mol to 80,000 g/mol or 30,000 g/mol to 60,000 g/mol.

The synthesis of a polymer of this type is described below in Example A2.

In another embodiment, the chemical entity comprises:

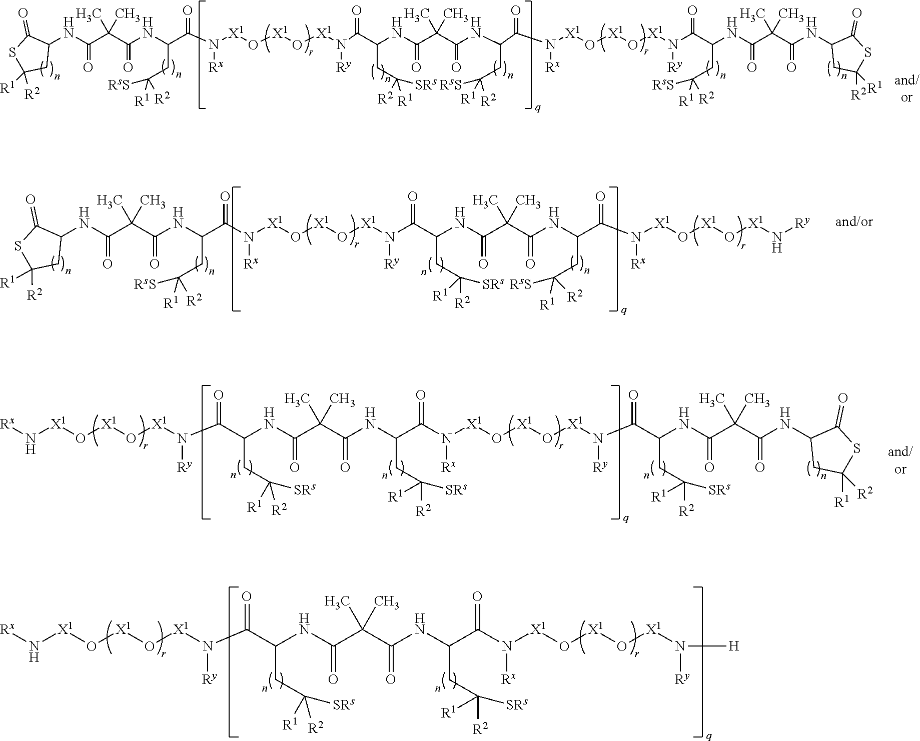

wherein n, X¹ and r are as defined herein;
R^{s} is H or N═O;
R^{x} and R^{y} are as defined above for formula (Xu) and
q is 10 to 1000, for example 15 to 500, 20 to 250 or 25 to 180.

The units

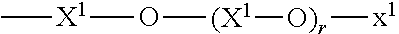

of this chemical entity are derived from a component C which is a compound of Formula (Xu) or a mixture of compounds of Formula (Xu).

In some cases, component C comprises or consists of a single compound of Formula (Xu). The compound of Formula (Xu) may be a compound of Formula (Xva) or (Xvb) as defined above in which r is from 2 to 10, for example 3 to 7 or 3.5 to 6. The synthesis of a polymer of this type is illustrated in Example A3 below.

The weight average molecular weight (M_{w}) of a polymer of this type may be from about 15,000 g/mol to 100,000 g/mol, for example 20,000 g/mol to 80,000 g/mol or 30,000 g/mol to 60,000 g/mol.

In other cases, Component C may be a mixture, for example component C may comprise or consist of two or more compounds of Formula (Xu). Suitably in this case, component C consists of a first compound of Formula (Xu) and a second compound of Formula (Xu).

The first compound of Formula (Xu) may be a compound of Formula (Xva) or (Xvb) as defined above in which r is from 2 to 10, for example 3 to 7 or 3.5 to 6.

The second compound of Formula (Xu) may be a compound in which not all X¹ groups are the same, for example some X¹ groups may be ethylene oxide units and other X¹ groups may be propylene oxide units. In this case r is suitably from about 11 to about 100, for example about 20 to about 60 or 30 to 50.

The synthesis of a polymer formed from the reaction of component B with a component C which comprises first and second compounds of Formula (Xu) is shown below in Example A4.

Typically, the weight average molecular weight (M_{w}) of a polymer of this type is from about 10,000 g/mol to 200,000 g/mol, for example 30,000 g/mol to 150,000 g/mol or 45,000 g/mol to 100,000 g/mol.

In a further embodiment, the chemical entity comprises:

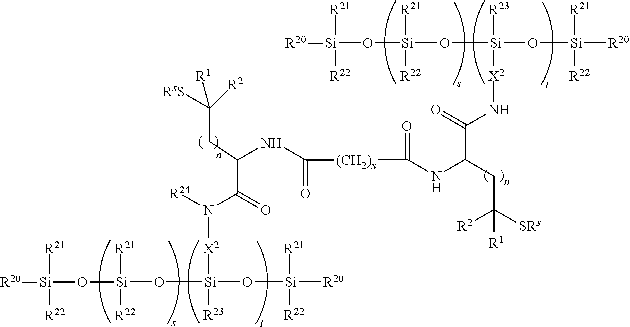

wherein R¹, R², R²⁰, R²¹, R²², R²³, n, X², s and t are as defined herein;
R^{s} is H or N═O; and
x is 1-10, suitably 1-6 and more suitably 1-4.

Some polymers of this type have a weight average molecular weight of about 50,000 to 200,000 g/mol, for example about 100,000 g/mol. In other cases, the polymers are cross-linked and have an infinite weight average molecular weight.

The synthesis of a polymer of this type is described in Example 18 below.

The chemical entities formed by reaction of component B with component C can be labile as defined infra. The labile nature of the entities typically results from one (or both) of components B and/or C comprising labile groups.

Thus, in one embodiment, the chemical entities (e.g. core moiety A (component B) and/or core moiety D (component C)) comprise moieties that are labile, for example moieties that are labile via hydrolysis or nucleophilic substitution, e.g. moieties containing one or more functional groups independently selected from the group consisting of an ester, an anhydride, an iminine, an imine, an acetal, a carbonate, a phosphazene, a phosphate ester, a urethane, a lactide and a lipolide.

In one embodiment, the chemical entities comprise a biocompatible, semi-crystalline, hydrolytically degradable polymer such as poly(dioxanone) (PDO), poly(glycolide) (PGA), poly(lactide) (PLA), poly(s-caprolactone), a poly(anhydride) such as poly(sebacic acid), a poly(hydroxyalkanoate) such as poly(3-hydroxybutyrate) (P30HB), or a copolymer of these polymers such as poly(glycolide)/trimethylene carbonate (PGA/TMC, e.g. 2:1 PGA/TMC), poly(lactide)/trimethylene carbonate (PLA/TMC) or poly(hydroxybutyrate/hydroxy valerate (PHB/PHV).

Moieties may also be labile via reduction-oxidation e.g. moieties containing one or more functional groups independently selected from the group consisting of an oxide, a superoxide, ozone, an oxoacid, an oxyacid, a halite, a hypohalite and a hypohalous.

Moieties may also be labile via photolysis (e.g. by UV or visible light) e.g. moieties containing one or more functional groups independently selected from the group consisting of a benzoyl carbonyl and an azide.

When the chemical entities are incorporated into a coating on a surface, depending on the extent of lability of the chemical entities, and the distribution of the chemical entities within a bulk substrate, or within a coating on a surface, the chemical entity/substrate/coating may degrade over time, or resorb over time.

In one embodiment, the chemical entity/substrate/coating is bioresorbable, meaning that the chemical entity/substrate/coating is substantially broken down by the in vivo environment in an amount of time of 1 to 48 months, e.g. 1 to 36 months, 1 to 24 months, 1 to 18 months or 1 to 12 months.

### Conversion of the Thiol (—SH) Form to S-Nitrosothiol (—SNO) Form

Chemical entities in thiol (—SH) form can be modified such that at least a proportion of the thiol groups are converted to S-nitrosothiol (—SNO) groups. Such reagents and techniques are well known to a person having ordinary skill in the art, and include, but are not limited to, reacting with aqueous nitric acid (e.g. formed in situ using sodium nitrite), or when the reaction is carried out in organic media, an alkyl nitrite such as t-butyl nitrite.

In one embodiment, the chemical entity has 20-100% of thiol (—SH) groups, such as 30-100%, 40-100%, 50-100% or 60-100% of free SH groups, relative to the molar % of component B.

In one embodiment, the chemical entity has 20-100% of S-nitrosothiol (—SNO) groups, such as 30-100%, 40-100%, 50-100% or 60-100% of free SH groups, relative to the molar % of component B.

### Medical Devices and Materials

The thiol-containing and S-nitrosothiol-containing chemical entities of the disclosure are of particular use in coating a surface of a medical device. In at least some embodiments, the coatings are uniform and uninterrupted (i.e. free of flaws) and/or are expected to have good adherence to surfaces and/or low brittleness.

The medical devices of the disclosure are suitable for a wide range of applications including, for example, a range of medical treatment applications within the body. Exemplary applications include use as a catheter balloon for transferring drug to, or placement of, or “touch-up” of implanted stents, stent-grafts or vascular grafts, use as stents, stent-grafts, catheters, a permanent or temporary prosthesis, or other type of medical implant, treating a targeted tissue within the body, and treating any body cavity, space, or hollow organ passage(s) such as blood vessels, the urinary tract, the intestinal tract, nasal or sinus cavities, neural sheaths, intervertebral regions, bone cavities, the oesophagus, intrauterine spaces, pancreatic and bile ducts, rectum, and those previously intervened body spaces that have implanted vascular grafts, stents, prosthesis, or other type of medical implants.

Additional examples of medical devices include indwelling monitoring devices, artificial heart valves (leaflet, frame, and/or cuff), pacemaker or defibrillator electrodes, guidewires, cardiac leads, sutures, embolic filters, vascular filters, cardiopulmonary bypass circuits, cannulae, plugs, drug delivery devices, tissue patch devices, blood pumps, patches, osteoprostheses, chronic infusion lines, arterial lines, devices for continuous subarachnoid infusions, feeding tubes, CNS shunts (e.g., a ventriculopleural shunt, a ventriculo-atrial (VA) shunt, or a ventriculoperitoneal (VP) shunt), ventricular peritoneal shunts, ventricular atrial shunts, portosystemic shunts and shunts for ascites, devices for the filtering or removal of obstructions such as emboli and thrombi from blood vessels, as a dilation device to restore patency to an occluded body passage, as an occlusion device to selectively deliver a means to obstruct or fill a passage or space (such as coils, embolic beads, liquid embolics) and as a centering mechanism for transluminal instruments like catheters. In one embodiment, the medical devices can be used to treat stent restenosis or treat tissue sites where previously placed drug-eluting constructs have failed. In another embodiment, medical devices as described herein can be used to establish, connect to, or maintain arteriovenous access sites, e.g., those used during kidney dialysis.

Further examples of medical devices which can be permanent or temporary are catheters. Examples of catheters include, but are not limited to, central venous catheters, peripheral intravenous catheters, haemodialysis catheters, catheters such as coated catheters include implantable venous catheters, tunnelled venous catheters, coronary catheters useful for angiography, angioplasty, or ultrasound procedures in the heart or in peripheral veins and arteries, hepatic artery infusion catheters, CVC (central venous catheters), peripheral intravenous catheters, peripherally inserted central venous catheters (PIC lines), flow-directed balloon-tipped pulmonary artery catheters, total parenteral nutrition catheters, chronic dwelling catheters (e.g., chronic dwelling gastrointestinal catheters and chronic dwelling genitourinary catheters), peritoneal dialysis catheters, CPB catheters (cardiopulmonary bypass), urinary catheters and microcatheters (e.g. for intracranial application). Catheters may be indwelling.

In one embodiment, the medical device is an expandable member. In another embodiment, the medical device is a balloon, a stent, a stent-graft or a graft.

Thus, in one embodiment, the medical device is an expandable member which can be a balloon, expandable catheter, stent, stent-graft, a self-expanding construct, a balloon expandable construct, a combination self-expanding and balloon expandable construct, a graft or a mechanical, radially expanding device which may be expanded, for example, via application of a torsional or longitudinal force. Expandable members can also include those which expand due to pneumatic or hydraulic pressure, those which expand due to magnetic forces, those which expand due to the application of energy (for example thermal, electrical, or ultrasonic (piezoelectric) energy). Expandable members can be placed temporarily in any lumen (e.g. a vessel) by expanding said device and then removed by collapsing said device by a torsional or longitudinal force. The chemical entities as described herein are of use in coating a surface of a device used in early cannulation. Thus, in one embodiment, the surface having a coating comprising chemical entities as described herein is an early cannulation graft. In another embodiment is provided an early cannulation graft comprising a surface having a coating comprising chemical entities as described herein.

In one embodiment, the medical device is a stent such as a bifurcated stent, balloon expandable stent or a self-expanding stent. Stents are configured as braids, wound wire forms, laser-cut forms, deposited materials, 3-D printed constructs, or combinations thereof, or take other structural forms, including those with length-adjustability, which provide support to a luminal wall or region. Stents are constructed of biocompatible materials including metals, metal alloys, such as stainless steel and nickel-titanium alloy (NiTi), polymers, ceramics, biodegradable materials (such as biodegradable polymers, ceramics, metals and metal alloys), or combinations thereof. Stents can be of substantially unitary form or comprise separate components, e.g., rings. Whether unitary or made up of components, stent structures can be joined together by struts, hinges, connectors, or materials which fully or partially line or cover the stent. In one embodiment, the stent structure is joined with fluoropolymers forming “webs” as described in US2009/0182413 (Gore Enterprise Holdings, Inc., incorporated herein by reference).

In one embodiment, the medical device is a stent such a bifurcated stent, a balloon expandable stent or a self-expanding stent. In one embodiment, the medical device is a stent formed from a metal, a metal alloy, a polymer, a ceramic, a biodegradable material, or a combination thereof.

In one embodiment, the medical device is a stent-graft. Stent-grafts combine at least one stent member with a graft component. Grafts are typically configured as tubular members, with closed walls or walls with openings. Graft materials include biocompatible materials such as fluoropolymers, including polytetrafluoroethylene (PTFE) and expanded polytetrafluoroethylene (ePTFE). Other suitable graft materials include polymers such as polyethylene terephthalate and ultra-high molecular weight polyethylene (UHMWPE). Graft materials can be made to possess different strengths, densities, dimensions, porosities and other functional characteristics and can take the form of films, extrusions, electrospun materials, coatings, depositions, or molded articles. Grafts may used alone or graft materials can fully or partially line or cover a stent structure. In one embodiment, the stent-graft can take forms as described in U.S. Pat. No. 5,876,432 (Gore Enterprise Holdings, Inc., incorporated herein by reference).

In one embodiment, the medical device is a stent graft, wherein the graft is formed from a polymer, suitably a biocompatible polymer. Suitably the graft is formed from a fluoropolymer such as expanded polytetrafluoroethylene (ePTFE). In one embodiment, the medical device is a graft.

Stents, stent-grafts and grafts can be overlain with various materials such as polymers and primer layers. In an embodiment, the stent or graft structure is modified to enhance the ability of the device to hold or release a therapeutic agent applied to the device. For example, pits or blind holes can be formed in stent struts into which a therapeutic agent is loaded. When coated onto a stent, stent-graft, or graft, the composition will release a therapeutic agent in a localized manner, therefore a stent, stent-graft or graft coated with a composition of the disclosure is referred to herein as a drug eluting stent (DES).

In one embodiment, the medical device is a medical balloon. Balloons useful in the embodiments of the present disclosure may be formed by using any conventional manner such as extrusion, blow molding and other molding techniques. Balloons may be compliant or semi-compliant or non-compliant and may be of various lengths, diameters, sizes and shapes. Balloons can be so called “conformable” or “conforming”, “length-adjustable” or “steerable” balloons. In other embodiments, the medical devices may comprise balloons which are constructed of wrapped films, are fiber-wound, are of variable length, are segmented, and/or have controlled or variable inflation profiles. In other embodiments, balloons may be overlain with a material or comprise more than one layer or be of composite construction. In an embodiment, the balloon surface or structure is modified to enhance the ability of the balloon to hold or release a therapeutic agent applied to it. For example, the balloon can be folded in such a way as to hold a therapeutic agent within said folds. When coated onto a balloon, the composition will release a therapeutic agent in a localized manner, therefore a balloon coated with a composition of the disclosure is referred to herein as a drug eluting balloon (DEB).

In one embodiment, the medical device is selected from the group consisting of stents including bifurcated stents, balloon expandable stents and self-expanding stents, stent-grafts including bifurcated stent-grafts, grafts including vascular grafts, bifurcated grafts and early cannulation grafts, dialators, vascular occluders, embolic filters, vascular filters, embolectomy devices, catheters including microcatheters, central venous catheters, peripheral intravenous catheters, indwelling catheters and hemodialysis catheters, artificial blood vessels, sheaths including retractable sheaths, blood indwelling monitoring devices, artificial heart valves, pacemaker electrodes, guidewires, cardiac leads, cardiopulmonary bypass circuits, cannulae, plugs, drug delivery devices, balloons, tissue patch devices, blood pumps, artificial pancreas devices, cell encapsulation devices, implantable sensor devices, embolic particles, drug releasing particles and liquid embolics.

In one embodiment, the medical device, in particular a surface of the medical device, is composed of a synthetic or naturally occurring organic or inorganic polymer or material, including but not limited to materials such as polyolefins, polyesters, polyurethanes, polyamides, polyether block amides, polyimides, polycarbonates, polyphenylene sulfides, polyphenylene oxides, polyethers, silicones, polycarbonates, polyhydroxyethylmethacrylate, polyvinyl pyrrolidone, polyvinyl alcohol, rubber, silicone rubber, polyhydroxyacids, polyallylamine, polyallylalcohol, polyacrylamide, and polyacrylic acid, styrenic polymers, polytetrafluoroethylene and copolymers thereof, expanded polytetrafluoroethylene and copolymers thereof, derivatives thereof and mixtures thereof, fluoropolymers such as VDF-HDF polymers, TFE-co-vinyl acetate or TFE-co-vinyl alcohol (see for example U.S. Pat. No. 10,688,188 and WO2021/097210 and WO2021/150962, both incorporated herein by reference). Some of these classes are available both as thermosets and as thermoplastic polymers. As used herein, the term “copolymer” shall be used to refer to any polymer formed from two or more monomers, e.g. 2, 3, 4, 5 and so on and so forth. Bioresorbables, such as poly(D,L-lactide) and polyglycolids and copolymers thereof are also useful. Non-woven, bioabsorbable web materials comprising a tri-block copolymer such as poly(glycolide-co-trimethylene carbonate) tri-block copolymer (PGA:TMC) are also useful (as described in U.S. Pat. No. 7,659,219; Biran et al., incorporated herein by reference). Useful polyamides include, but are not limited to, nylon 12, nylon 11, nylon 9, nylon 6/9 and nylon 6/6. Examples of some copolymers of such materials include the polyether-block-amides, available from Elf Atochem North America in Philadelphia, Pa. under the tradename of PEBAX®. Another suitable copolymer is a polyetheresteramide. Suitable polyester copolymers, include, for example, polyethylene terephthalate and polybutylene terephthalate, polyester ethers and polyester elastomer copolymers such as those available from DuPont in Wilmington, Del. under the tradename of HYTREL® Block copolymer elastomers such as those copolymers having styrene end blocks, and midblocks formed from butadiene, isoprene, ethylene/butylene, ethylene/propene, and so forth may be employed herein. Other styrenic block copolymers include acrylonitrile-styrene and acrylonitrile-butadiene-styrene block copolymers. Also, block copolymers wherein the particular block copolymer thermoplastic elastomers in which the block copolymer is made up of hard segments of a polyester or polyamide and soft segments of polyether may also be employed herein. Other useful materials are polystyrenes, poly(methyl)methacrylates, polyacrylonitriles, poly(vinylacetates), poly(vinyl alcohols), chlorine-containing polymers such as poly(vinyl) chloride, polyoxymethylenes, polycarbonates, polyamides, polyimides, polyurethanes, phenolics, amino-epoxy resins, polyesters, silicones, cellulose-based plastics, and rubber-like plastics. Combinations of these materials can be employed with and without cross-linking. Polymeric materials may optionally be blended with fillers and/or colorants, such as a gold, barium, or tantalum filler to render the polymeric material radiopaque. Polymeric materials may optionally be modified at their surface while retaining bulk properties using methods known in the art, such as acid or base etching, hydrolysis, aminolysis, plasma modification, plasma grafting, corona discharge modification, chemical vapour deposition, ion implantation, ion sputtering, ozonation, photomodification, electron beam modification, gamma beam modification, and the like.

In an embodiment, a surface of the medical device is composed of nylon.

In one embodiment, the medical device, in particular a surface of the medical device is biocompatible and comprises a polyether-block-amide, such as Pebax®.

The medical device, in particular a surface of the medical device, may be composed of one or more fluorinated polymers such as fluoropolymers, e.g. expanded polytetrafluoroethylene (ePTFE), polytetrafluoroethylene (PTFE), fluorinated ethylene-propylene (FEP), perfluorocarbon copolymers, e.g. tetrafluoroethylene perfluoroalkylvinyl ether (TFE/PAVE) copolymers, copolymers of tetrafluoroethylene (TFE) and perfluoromethyl vinyl ether (PMVE), copolymers of TFE with functional monomers that comprise acetate, alcohol, amine, amide, sulfonate, functional groups and the like as described in U.S. Pat. No. 8,658,707 (W. L. Gore and Associates, incorporated herein by reference, as well as combinations thereof (see also U.S. Pat. No. 10,688,188 and WO2021/097210 and WO2021/150962, all incorporated herein by reference) and VDF-HFP. Also contemplated are combinations of the above with and without crosslinking between the polymer chains, expanded polyethylene, polyvinylchloride, polyurethane, silicone, polyethylene, polypropylene, polyurethane, polyglycolic acid, polyesters, polyamides, elastomers and their mixtures, blends and copolymers or derivatives thereof. ePTFE has a porous microstructure which is particularly compatible with the coating of the disclosure. Suitably a surface of the medical device is composed of ePTFE.

As used herein, the term “porous” refer to a material having openings, for example spaces (or pores) between ePTFE nodes and fibrils. Usually, as in the case of ePTFE, the pores of a porous material contain air when the material is not “wetted” i.e. when the material is not in contact with a liquid that fills the pores and displaces the air. The porosity of a device composed of ePTFE can be evaluated using various methods and parameters, as described in US2013/0231733 (W.L. Gore & Associates, Inc., incorporated herein by reference).

The medical device, in particular a surface of the medical device, may also be composed of one or more metals, including, but are not limited to, biocompatible metals, titanium, stainless steel, high nitrogen stainless steel, gold, silver, rhodium, zinc, platinum, rubidium, copper and magnesium, and combinations thereof. Suitable alloys include cobalt alloys including cobalt-chromium alloys such as L-605, MP35N, Elgiloy, titanium alloys including nickel-titanium alloys (such as Nitinol), tantalum, and niobium alloys, such as Nb-1% Zr, and others. In one embodiment, the medical device is a stent and is composed of biocompatible metal selected from stainless steel, tantalum, titanium alloys and cobalt alloys. The medical device, in particular a surface of the medical device may also be composed of a ceramic substrate including, but are not limited to, silicone oxides, aluminum oxides, alumina, silica, hydroxyapatites, glasses, calcium oxides, polysilanols, and phosphorous oxide.

In one embodiment, the medical device is covered with a porous material onto which a coating layer of the present disclosure is applied. In one embodiment, at least a portion of the surface of the device being coated is porous. In an embodiment, the medical device covering material is a fluoropolymer such as polytetrafluoroethylene (PTFE) or an expanded PTFE (ePTFE). The structure of expanded PTFE characterized by nodes interconnected by fibrils, is taught in U.S. Pat. Nos. 3,953,566 and 4,187,390 (W. L. Gore & Associates; both incorporated herein by reference). In one embodiment, the fluoropolymer medical device covering comprises ePTFE having a material structure with fibrils or fibrils and nodes. In another embodiment, the fibrils or fibrils and nodes change in size, dimension, or orientation as a dimension of the expandable member covering is changed. In one embodiment, the medical device is a balloon, disposed over at least a part of which is a covering, the covering being made at least in part of ePTFE, and disposed over at least a portion of the ePTFE balloon covering is a coating of the present disclosure.

In one embodiment, the medical device comprises a covering disposed around at least a portion of a coating layer of the disclosure. Such a covering may also be described as a sheath. In one embodiment the covering is removable from over the coating layer. In one embodiment, the covering is disposed over a coating layer of the disclosure applied to an expandable member. The covering can comprise any biocompatible material, including any possessing porosity or permeability. In one embodiment, the porosity or permeability varies as the material is deformed or otherwise altered in dimension.

Materials which may exhibit porosities or permeabilities that change with changes in the dimension of covering include, but are not limited to, fibrillated structures, such as expanded fluoropolymers (for example, expanded polytetrafluoroethylene (ePTFE)) or expanded polyethylene (as described in U.S. Pat. No. 6,743,388 (Sridharan et al.) and incorporated herein by reference); fibrous structures (such as woven or braided fabrics; non-woven mats of fibers, microfibers, or nanofibers; materials made from processes such as electrospinning or flash spinning; polymer materials comprising or consisting of melt or solution processable materials such as fluoropolymers, polyamides, polyurethanes, polyolefins, polyesters, polyglycolic acid (PGA), polylactic acid (PLA), and trimethylene carbonate (TMC), and the like; films with openings created during processing (such as laser- or mechanically-drilled holes); open cell foams; microporous membranes made from materials such as fluoropolymers, polyamides, polyurethanes, polyolefins, polyesters, PGA, PLA, TMC, and the like; porous polyglycolide-co-trimethylene carbonate (PGA:TMC) materials (as described in U.S. Pat. No. 8,048,503 (Gore Enterprise Holdings, Inc.)) and incorporated herein by reference); or combinations of the above. Processing of the above materials may be used to modulate, enhance or control porosity or permeability between a first, closed state and second, more porous or permeable state. Such processing may help close the material structure (thus lowering porosity or permeability) in a first state, help open the material structure in a second state, or a combination of both. Such processing which may help close the material structure may include, but is not limited to: calendaring, coating (discontinuously or continuously), compaction, densification, coalescing, thermal cycling, or retraction and the like. Such processing that may help open the material structure may include, but is not limited to: expansion, perforation, slitting, patterned densification and/or coating, and the like. In another embodiment, said materials comprise pores between fibrils or between nodes interconnected by fibrils, such as in ePTFE.

One skilled in the art will appreciate various methods which characterize the change in porosity or permeability using testing at a first state comparing to testing done at a second state. These methods include, but are not limited to, characterizations of air or liquid flux across the material structure at a given pressure differential, characterization which determines the pressure differential at which different fluids strike through the material structure such as Water Entry Pressure or Bubble Point, and visual characterization as measured from an image (e.g. from a scanning electron microscope or light microscope).

In one embodiment, the covering material is a fluoropolymer such as expanded polytetrafluoroethylene (ePTFE), polytetrafluoroethylene (PTFE), fluorinated ethylene-propylene (FEP), perfluorocarbon copolymers, e.g. tetrafluoroethylene perfluoroalkylvinyl ether (TFE/PAVE) copolymers, copolymers of tetrafluoroethylene (TFE) and perfluoromethyl vinyl ether (PMVE), or copolymers of TFE with functional monomers that comprise acetate, alcohol, amine, amide, sulfonate, functional groups and the like as described in U.S. Pat. No. 8,658,707 (W. L. Gore and Associates, incorporated herein by reference), as well as combinations thereof. In another embodiment, the fluoropolymer covering possesses a material structure which changes as a dimension of the covering changes. In one embodiment, the fluoropolymer covering comprises ePTFE having a material structure with fibrils or fibrils and nodes. In another embodiment, the fibrils or fibrils and nodes change in size, dimension, or orientation as a dimension of the covering is changed. In one embodiment, the medical device is a balloon, disposed over at least a part of which is a covering, the covering being made at least in part of ePTFE, and the material structure of the ePTFE changes upon expansion of the balloon.

In another embodiment, the medical device is a balloon, disposed over at least a part of which is a coating layer of the disclosure which in turn is covered at least in part with a covering such as a sheath, the covering being made at least in part of ePTFE, and the material structure of the ePTFE changes upon expansion of the balloon.

In one embodiment the covering is essentially hydrophobic and is treated to render it hydrophilic using, for example, the methods described in US2013/0253426 (W. L. Gore & Associates; incorporated herein by reference). In another embodiment, the covering comprises a film or film tube of ePTFE.

In another embodiment, the surface(s) or outward configuration of the covering material may be modified with textures, protrusions, wires, blades, spikes, scorers, depressions, grooves, coatings, particles, and the like. In another embodiment, the surface(s) or outward configuration of the covering material may be modified with needles, cannulae, and the like. These modifications may serve various purposes such as to modify tissues into which therapeutic agents will be (or have been) delivered, control placement of the system of the disclosure, and direct fluid transfer. Such textures may help in increased transfer of a therapeutic agent onto, more deeply and/or into deeper tissues. Such textures may be comprised of the covering material, or may be comprised of an added material.

In another embodiment, the location(s) of the permeable microstructure may be varied. For example, a covering may be constructed such that only a portion of its microstructure is variably permeable. Such a configuration may be desirable where fluid transfer is not desired to occur, for example, at one or both of the ends of the expandable medical device of the disclosure. This may be desirable where multiple drug eluting devices will be used in a specific anatomy, and it would be undesirable to overlap treatments sites, i.e., delivering too much drug to a particular site.

In another embodiment, the covering may contain or be marked with radiopaque markers or be constructed to be radiopaque in its entirety. Such radiopaque indicators are used by clinicians to properly track and place an expandable medical device of the disclosure.

The coating of the disclosure can be applied to the entire surface of the medical device, or only a portion of the surface of the medical device. Certain devices may have an external surface and an internal surface, either or both of which can be coated. For example, tubular substrates including but not limited to artificial blood vessels, vascular grafts, stents, and stent grafts, have an internal surface, or lumen, which can be coated independently from the external surface. A device comprising an internal and an external surface may only require the external surface to be coated. Conversely, only the internal surface may require a coating. In one embodiment, the amount or thickness of the coating may be varied over the surface of the medical device. The coating layer can be continuous over an entire surface of the device or be discontinuous and cover only a portion or separate portions of the device. The coating layer can also be “sculpted” or modified to create a desired surface topography or modified with textures, as described supra.

In one embodiment, the coating also can be applied to a medical device in the form of a liquid (U.S. Pat. No. 10,688,188, WO2021/097210, both incorporated herein by reference), or as nanoparticles (WO2021/150962, incorporated herein by reference).

In one embodiment, up to 99%, for example up to 95%, 90%, 75%, 50% or 25% of the surface area of the medical device is coated with the chemical entities of the disclosure. In one embodiment, both the external and internal surfaces of the medical device are coated. In another embodiment, only the external surface of the medical device is coated.

The surface having a coating comprising the chemical entities described herein can comprise one or more additional coatings. In one embodiment, the additional coatings are selected from the group consisting of a synthetic or naturally occurring organic or inorganic polymer or material, such as polyolefins, polyesters, polyurethanes, polyamides, polyether block amides, polyimides, polycarbonates, polyphenylene sulfides, polyphenylene oxides, polyethers, silicones, polycarbonates, polyhydroxyethylmethacrylate, polyvinyl pyrrolidone, polyvinyl alcohol, rubber, silicone rubber, polyhydroxyacids, polyallylamine, polyallylalcohol, polyacrylamide, polyacrylic acid, styrenic polymers, fluoropolymers, (e.g. expanded polytetrafluoroethylene (ePTFE), polytetrafluoroethylene (PTFE), fluorinated ethylene-propylene (FEP), perfluorocarbon copolymers (e.g. tetrafluoroethylene perfluoroalkylvinyl ether (TFE/PAVE) copolymers, copolymers of tetrafluoroethylene (TFE) and perfluoromethyl vinyl ether (PMVE)), copolymers of TFE with functional monomers that comprise acetate, alcohol, amine, amide, sulfonate, functional groups and the like as described in U.S. Pat. No. 8,658,707 (W. L. Gore and Associates, incorporated herein by reference, as well as combinations thereof (see also U.S. Pat. No. 10,688,188 and WO2021/097210 and WO2021/150962, all incorporated herein by reference) and VDF-HFP), bioresorbables, such as poly(D,L-lactide), polyglycolids and copolymers thereof, non-woven, bioabsorbable web materials comprising a tri-block copolymer such as poly(glycolide-co-trimethylene carbonate) tri-block copolymer (PGA:TMC), nylon 12, nylon 11, nylon 9, nylon 6/9 and nylon 6/6, polyetheresteramide, polyethylene terephthalate and polybutylene terephthalate, polyester ethers, polyester elastomer copolymers, block copolymer elastomers such as those copolymers having styrene end blocks, and midblocks formed from butadiene, isoprene, ethylene/butylene, ethylene/propene, styrenic block copolymers including acrylonitrile-styrene and acrylonitrile-butadiene-styrene block copolymers, polystyrenes, poly(methyl)methacrylates, polyacrylonitriles, poly(vinylacetates), poly(vinyl alcohols), chlorine-containing polymers such as poly(vinyl) chloride, polyoxymethylenes, polycarbonates, polyamides, polyimides, polyurethanes, phenolics, amino-epoxy resins, polyesters, silicones, cellulose-based plastics, rubber-like plastics, expanded polyethylene, polyvinylchloride, polyurethane, silicone, polyethylene, polypropylene, polyurethane, polyglycolic acid, polyesters, polyamides, elastomers and their mixtures, blends and copolymers or derivatives.

At least one of the one or more additional coatings can comprise a therapeutic agent, e.g. selected from the group consisting of an antithrombogenic agent, an anti-calcification agent, a hemostatic agent, an anti-angiogenic agent, an angiogenic agents, an anti-microbial agent, an anti-proliferative agent, a proliferative agent and an anti-inflammatory agent, or a combination thereof.

In one embodiment, at least one of the one or more additional coatings comprise a therapeutic agent selected from cilostazol, an mTOR inhibitor (e.g. sirolimus, zotarolimus, tacrolimus or everolimus), dicumarol, dicumarol complexed with an appropriate cyclodextrin, carvedilol, an antithrombogenic agent (e.g. heparin, a heparin derivative, a urokinase, or dextrophenylalanine) proline, polyproline, arginine, polyarginine, lysine, polylysine, chloromethylketone; an antiinflammatory agent (e.g. dexamethasone, prednisolone, corticosterone, budesonide, estrogen, sulfasalazine or mesalamine), an anti-neoplastic/antiproliferative/anti-miotic agent (e.g. a major taxane domain-binding drug, such as paclitaxel or an analogue thereof, epothilone, discodermolide, docetaxel, paclitaxel protein-bound particles such as ABRAXANE® (ABRAXANE is a registered trademark of ABRAXIS BIOSCIENCE, LLC), paclitaxel complexed with an appropriate cyclodextrin (or cyclodextrin like molecule)), rapamycin, a rapamycin analog, rapamycin complexed with an appropriate cyclodextrin or cyclodextrin like molecule, 17p-estradiol, 17p-estradiol complexed with an appropriate cyclodextrin, p-lapachone or an analog thereof, 5-fluorouracil, cisplatin, vinblastine, cladribine, vincristine, an epothilone, endostatin, angiostatin, angiopeptin, a monoclonal antibody capable of blocking smooth muscle cell proliferation, a thymidine kinase inhibitor; a lytic agent; an anaesthetic agent (e.g. lidocaine, bupivacaine or ropivacaine); an anti-coagulants (e.g. D-Phe-Pro-Arg chloromethyl ketone, an RGD peptide-containing compound, AZX100 a cell peptide that mimics HSP20 (Capstone Therapeutics Corp., USA), heparin, hirudin, an antithrombin compound, a platelet receptor antagonist, an anti-thrombin antibody, an anti-platelet receptor antibody, aspirin, a prostaglandin inhibitor, a platelet inhibitor or a tick antiplatelet peptide), a vascular cell growth promoter such as a growth factor, a transcriptional activator, a translational promoter, a vascular cell growth inhibitor, a growth factor inhibitor, a growth factor receptor antagonist, a transcriptional repressor, a translational repressor, a replication inhibitor, an inhibitory antibody, an antibody directed against growth factors, a functional molecule consisting of a growth factor and a cytotoxin, a functional molecule consisting of an antibody and a cytotoxin, a protein kinase and tyrosine kinase inhibitor (e.g. a tyrphostin, genistein, or a quinoxaline), a prostacyclin analog; a cholesterol-lowering agent; an angiopoietin; an antimicrobial agent (e.g. triclosan, a cephalosporin, an aminoglycoside or nitrofurantoin), a cytotoxic agent, a cytostatic agent, a cell proliferation affector, a vasodilating agent, an agent that interferes with endogenous vasoactive mechanisms, an inhibitor of leukocyte recruitment (e.g. a monoclonal antibody or cytokine), a hormone, a radiopaque agent (e.g. an iodinated contrast agent), gold, or barium, or a combination thereof. Suitably, an additional coating comprises an antithrombogenic agent such as heparin or a heparin derivative. In one embodiment, an additional coating is the Carmeda® BioActive Surface (CBAS®).

In one embodiment, the additional coating(s) is/are selected from the group consisting of fluoropolymers and antithrombogenic agents. In one embodiment, the additional coating(s) is/are selected from the group consisting of expanded polytetrafluoroethylene (ePTFE), polytetrafluoroethylene (PTFE), fluorinated ethylene-propylene (FEP), perfluorocarbon copolymers (e.g. tetrafluoroethylene perfluoroalkylvinyl ether (TFE/PAVE) copolymers, copolymers of tetrafluoroethylene (TFE) and perfluoromethyl vinyl ether (PMVE)), copolymers of TFE with functional monomers that comprise acetate, alcohol, amine, amide, sulfonate, functional groups and the like as described in U.S. Pat. No. 8,658,707 (W. L. Gore and Associates, incorporated herein by reference, as well as combinations thereof (see also U.S. Pat. No. 10,688,188 and WO2021/097210 and WO2021/150962, both incorporated herein by reference), VDF-HFP, heparin, and a heparin derivative.

In one embodiment, the additional coating(s) is/are applied as a first coating on a surface, wherein the chemical entities are applied as a second coating on a surface, e.g. at least a proportion of, or all of the chemical entities are coated on top of the first coating. In an alternative embodiment, the chemical entities are applied as a first coating on a surface, wherein the additional coating(s) is/are applied as a second coating on the surface, e.g. at least a proportion of, or all of the additional coating(s) is/are applied on top of the first coating of chemical entities. Alternatively, the additional coating(s) can be mixed with the chemical entities prior to application to the surface, e.g. as described in Examples 26-29, a fluoropolymer (VDF-HFP fluoropolymer) is mixed with chemical entities, and the mixture is then applied to the surface (e.g. ePTFE film, stainless steel foil, Pebax® nylon film, or ePE film).

When the additional coating is applied as a second coating on top of the coating comprising the chemical entities, it is expected that nitric oxide release will still occur, as due to the low molecular weight and small molecular size of the NO molecule it is expected to diffuse, permeate, or otherwise pass through the additional coating(s).

In this regard, an additional coating (e.g. a fluoropolymer such as VDF-HFP) incorporated on top of the coating of chemical entities, or applied in a mixture with the chemical entities can be effective at delaying the onset of release of NO from the surface e.g. as shown in Examples 26-29.

The surface having a coating comprising chemical entities can be sterilized, in particular when the surface is the surface of a medical device. Suitable sterilization processes include, but are not limited to, sterilization using ethylene oxide, vapour hydrogen peroxide, plasma phase hydrogen peroxide, dry heat, autoclave steam sterilization, chlorine dioxide sterilization, gamma ray sterilization or electron beam sterilization. Sterilization using ethylene oxide is the most commonly utilized, proven and readily available sterilization technique for implantable medical devices such as stents, stent grafts, balloons, indwelling catheters and balloon catheters. Thus, in one embodiment, a coating comprising chemical entities is stable to sterilization, in particular ethylene oxide sterilization. Thus, in one embodiment is provided a coated medical device as described herein which has been sterilized, e.g. ethylene oxide sterilized. In another embodiment is provided chemical entities as described herein which have been sterilized, e.g. ethylene oxide sterilized. Representative Evaluation Method B includes a method of ethylene oxide sterilization, and a method of assessing stability post-sterilization.

In one embodiment, at least 80%, such as at least 85%, 90% or 95% of NO release activity is retained following sterilization using Evaluation Method B.

In one embodiment, the chemical entities are free of polysiloxane.

### Therapeutic Methods

Chemical entities of the disclosure comprising S-nitrosothiol groups, and devices, in particular medical devices coated with the chemical entities comprising S-nitrosothiol groups are of use in medical therapy, wherein the chemical entities/coating comprising the chemical entities releases nitric oxide.

In one embodiment is provided a medical device with a surface having a coating layer comprising chemical entities comprising S-nitrosothiol groups as described hereinabove for use in a method of treating tissue in the human or animal body, wherein the chemical entities/coating comprising the chemical entities releases nitric oxide. The tissue to be treated includes any body cavity, space, or hollow organ passage(s) such as blood vessels, a fistula such as an arteriovenous fistula (AVF), lymphatic vessels, the urinary tract, the intestinal tract, nasal cavity, neural sheath, intervertebral regions, bone cavities, esophagus, intrauterine spaces, pancreatic and bile ducts, renal glomeruli, hepatic sinusoids, rectum, and those previously intervened body spaces that have implanted vascular grafts, stents, prosthesis, or other type of medical implants.

The medical device with a surface having a coating layer as described herein can be of use in the removal of obstructions such as emboli and thrombi from blood vessels, as a dilation device to restore patency to an occluded body passage, as an occlusion device to selectively deliver a means to obstruct or fill a passage or space, and as a centering mechanism for transluminal instruments like catheters.

In one embodiment is provided a chemical entity as described hereinabove for use in preventing or treating a condition which benefits from the delivery of nitric oxide.

In one embodiment is provided a chemical entity as described hereinabove for use in opening blood vessels and/or improving oxygen levels.

In another embodiment is provided a chemical entity as described hereinabove for use in the preventing or treating an atherosclerotic lesion.

In another embodiment is provided a chemical entity as described hereinabove for use in the preventing or treating an hypervascular lesion, such as an angioma, a hypervascular tumour.

In another embodiment is provided a chemical entity as described hereinabove for use in the preventing or treating angiogenesis (including vasculogenesis).

In another embodiment is provided a chemical entity as described hereinabove for use in promoting endothelialization, in particular of grafts/stent-grafts/stents.

In another embodiment is provided a chemical entity as described hereinabove for use in preventing or treating infection, in particular bacterial infection.

In another embodiment is provided a chemical entity as described hereinabove for use in maturing an arteriovenous fistula.

In another embodiment is provided a chemical entity as described hereinabove for use in preventing or treating a skin ulcer, e.g. a foot ulcer.

In another embodiment is provided a chemical entity as described hereinabove for use in acceleration of healing from the inflammation cycle.

In all of the above embodiments utilizing a chemical entity as described herein, the chemical entity can be administered as a pharmaceutical composition. Thus, in one embodiment, is provided a pharmaceutical composition comprising a chemical entity as described herein and one or more pharmaceutically acceptable diluents or carriers, including, but not limited to, tonicity agents, buffers, surfactants, stabilizing polymers, preservatives, co-solvents and/or viscosity building agents.

The chemical entity can be administered by any convenient method, e.g. by oral, parenteral, buccal, sublingual, nasal, rectal, intrathecal or transdermal administration, and the pharmaceutical compositions adapted accordingly.

The chemical entity can be administered topically to the target organ, e.g. the chemical entities can be formulated as solutions, suspensions, emulsions, ointments, gels or patches.

A suspension or solution of a chemical entity as described herein will contain a suitable liquid carrier(s). Typical parenteral compositions consist of a solution or suspension of the chemical entity in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

The chemical entity can also be administered by injection, e.g. by subcutaneous or intramuscular injection or by intravenous injection or infusion. Suitable diluents for injection include isotonic saline, isotonic dextrose, and water (e.g. sterile water for injection or bacteriostatic water for injection).

In another embodiment is provided a method of preventing or treating a condition which benefits from the delivery of nitric oxide, comprising administering a chemical entity as described herein to a subject in need thereof.

In another embodiment is provided a method for opening blood vessels and/or improving oxygen levels, comprising administering a chemical entity as described herein to a subject in need thereof.

In another embodiment is provided a method for the preventing or treating an atherosclerotic lesion, comprising administering a chemical entity as described herein to a subject in need thereof.

In another embodiment is provided a method for the preventing or treating an hypervascular lesion, such as an angioma, a hypervascular tumour, comprising administering a chemical entity as described herein to a subject in need thereof.

In another embodiment is provided a method for the preventing or treating angiogenesis (including vasculogenesis), comprising administering a chemical entity as described herein to a subject in need thereof.

In another embodiment is provided a method for promoting endothelialization, in particular of grafts/stent-grafts/stents, comprising administering a chemical entity as described herein to a subject in need thereof.

In another embodiment is provided a method for preventing or treating infection, in particular bacterial infection, comprising administering a chemical entity as described herein to a subject in need thereof.

In another embodiment is provided a method for maturing an arteriovenous fistula, comprising administering a chemical entity as described herein to a subject in need thereof.

In another embodiment is provided a method for preventing or treating a skin ulcer, e.g. a foot ulcer, comprising administering a chemical entity as described herein to a subject in need thereof.

In another embodiment is provided a method for acceleration of healing from the inflammation cycle, comprising administering a chemical entity as described herein to a subject in need thereof.

In one embodiment is provided a method for preventing or treating a condition which benefits from the delivery of nitric oxide, where a coated medical device as described herein is used. Suitably the coated medical device is implanted or in contact with the area to be treated, i.e. the area benefiting from the delivery of nitric oxide.

In another embodiment is provided a method for opening blood vessels and/or improving oxygen levels, where a coated medical device as described herein is used.

In another embodiment is provided a method for preventing or treating an atherosclerotic lesion, where a coated medical device as described herein is used.

In another embodiment is provided a method for preventing or treating an hypervascular lesion, such as an angioma, a hypervascular tumour, where a coated medical device as described herein is used.

In another embodiment is provided a method for preventing or treating angiogenesis, where a coated medical device as described herein is used. In these embodiments, the treating of angiogenesis may comprise inducing vasculogenesis about or within a coated medical device such as an implantable cell encapsulation device, an implantable sensor device, an implantable drug delivery device, and the like.

In another embodiment is provided a method for promoting endothelialization, in particular of grafts/stent-grafts/stents, where a coated medical device as described herein is used.

In another embodiment is provided a method for preventing or treating infection, in particular bacterial infection, where a coated medical device as described herein is used.

In another embodiment is provided a method for maturing an arteriovenous fistula, where a coated medical device as described herein is used. Suitably, the medical device coated with a chemical entity as described herein is a sandwich construct such as the Gore® Acuseal® vascular graft. In another embodiment is provided a method for preventing or treating a skin ulcer, e.g. a foot ulcer, where a coated medical device as described herein is used. Suitably, the medical device is a dressing or filler material coated with a chemical entity as described herein. Suitable dressings include hydrogel dressing and hydrocolloid dressings. Suitable fillers include but are not limited to synthetic biodegradable polymers, such as poly(dioxanone) (PDO), poly(glycolide) (PGA), poly(lactide) (PLA), poly(s-caprolactone), a poly(anhydride) such as poly(sebacic acid), a poly(hydroxyalkanoate) such as poly(3-hydroxybutyrate) (P30HB), or a copolymer of these polymers such as poly(glycolide)/trimethylene carbonate (PGA/TMC, e.g. 2:1 PGA/TMC), poly(lactide)/trimethylene carbonate (PLA/TMC) or poly(hydroxybutyrate/hydroxy valerate (PHB/PHV).

In another embodiment is provided a method for acceleration of healing from the inflammation cycle, where a coated medical device as described herein is used.

In one embodiment, the coating is an antibacterial coating.

In all therapeutic aspects and embodiments above, the chemical entities/coating comprising the chemical entities release nitric oxide.

### Methods for Preparing Chemical Entities and Coatings of the Disclosure

As discussed above, the chemical entities of the disclosure in thiol form are formed by reaction of components B and C. Thus, in one embodiment is provided a process comprising reacting components B and C as defined herein.

Suitably, the process is carried out by dissolving components B and C in an organic anhydrous solvent (e.g. chloroform, THF, ethanol or DMSO, or mixtures thereof). Suitable procedures for forming chemical entities of the disclosure are described in Examples 9-18.

As discussed above, the chemical entities in thiol form can be converted to the S-nitrosothiol form using reagents and techniques that are well known to a person having ordinary skill in the art.

As discussed above, chemical entities of the disclosure (in thiol form or in S-nitrosothiol form) can be applied to a surface to form a coating. Thus, in one embodiment is provided a method of forming a coating on a surface, wherein said method comprises the step of:
(a) contacting the surface with the chemical entity as defined herein.

Step (a) is suitably carried out by step (a-1) dissolving the chemical entity in a solvent to form a solution, and then step (a-2) coating the surface with the solution. The solution can be applied to the surface by a variety of methods e.g. pipetting, dipping, spraying, casting, rolling or brushing. Spray coating is a particularly suitable method. Solvents which may be used include water, aqueous buffer (e.g. PBS), acetone, alcohols (such as methanol, ethanol, propanol, isopropanol), THF, DMF, DMSO, EtOAc, dioxane, acetonitrile, chloroform, NMP or mixtures thereof. In at least some embodiments, the chemical entities are soluble in solvents which are benign and/or suitable in bulk manufacturing settings e.g. water, alcohols and acetone.

Following step (a-2), a drying step (a-3) may be required to remove the solvent. The drying may be, for example, via localized heating, or air drying, or drying under a gas stream (such as under argon). The drying step may be made considerably easier when solvents such as acetone, alcohols and water can be used in step (a-1).

Examples of this method are described in Example 25.

If step (a) (steps (a-1) and (a-2) and optionally (a-3)) is carried out using chemical entities in thiol form, then the method can further comprise step (b) of converting at least a proportion of the thiol (—SH) groups in the chemical entity coating to S-nitrosothiol (—SNO) groups. Step (b) therefore comprises reacting the surface of step (a), (a-2) or (a-3) under conditions such that at least a proportion of the thiol groups are converted to S-nitrosothiol groups, such conditions being well known to a person having ordinary skill in the art.

Under certain circumstances, in addition to forming a coating of a chemical entity as defined herein, it may be beneficial to include one or more additional coatings, as described hereinabove.

The additional coating(s) can be mixed with the chemical entities prior to application to the surface. Thus, in one embodiment is provided a method of forming a coating on a surface, wherein said method comprises the steps of:
(a) forming a mixture of a chemical entity as defined herein and an additional coating component;
(b) contacting the surface with the mixture of step (a).

Step (a) is suitably carried out by dissolving the chemical entity and additional coating component(s) in a solvent to form a solution, which in step (b) is then coated onto the surface. The solution can be applied to the surface as described in the method directly above. Solvents which may be used are as described in the method directly above. Suitable procedures for coating surfaces with a mixture of a chemical entity of the disclosure and an additional coating are described in Examples 26-29.

Following step (b), a drying step (b-1) may be required to remove the solvent. The drying may be, for example, via localized heating, or air drying, or drying under a gas stream (such as under argon).

If steps (a) and (b) (or step (b-1)) are carried out using chemical entities in thiol form, then the method can further comprise step (c) of converting at least a proportion of the thiol (—SH) groups in the chemical entity coating to S-nitrosothiol (—SNO) groups. Step (c) therefore comprises reacting the surface of step (b) or (b-1) under conditions such that at least a proportion of the thiol groups are converted to S-nitrosothiol groups, such conditions being well known to a person having ordinary skill in the art.

Alternatively, the additional coating(s) can be applied to the surface before chemical entities are applied to the surface. Thus, in one embodiment is provided a method of forming a coating on a surface, wherein said method comprises the steps of:
(a) contacting the surface with an additional coating component;
(b) contacting the surface of step (a) with the chemical entity as defined herein.

Step (a) can be carried out using any known coating method which is suitable for the particular additional coating(s). Step (b) is suitably carried out by step (b-1) dissolving the chemical entity in a solvent to form a solution, and then step (b-2) coating the surface of step (a) with the solution. The solution can be applied to the surface as described in the methods directly above. Solvents which may be used are as described in the methods directly above.

Following steps (a) and/or (b), a drying step may be required to remove the solvent. The drying may be, for example, via localized heating, or air drying, or drying under a gas stream (such as under argon).

If step (b) is carried out using chemical entities in thiol form, then the method can further comprise step (c) of converting at least a proportion of the thiol (—SH) groups in the chemical entity coating to S-nitrosothiol (—SNO) groups. Step (c) therefore comprises reacting the surface of step (b) under conditions such that at least a proportion of the thiol groups are converted to S-nitrosothiol groups, such conditions being well known to a person having ordinary skill in the art.

Alternatively, the additional coating(s) can be applied to the surface after the chemical entities have been applied to the surface. Thus, in one embodiment is provided a method of forming a coating on a surface, wherein said method comprises the steps of:
(a) contacting the surface with a chemical entity as described herein;
(b) contacting the surface of step (a) with the additional coating component(s).

Step (a) is suitably carried out by step (a-1) dissolving the chemical entity in a solvent to form a solution, and then step (a-2) coating the surface of step (a) with the solution. The solution can be applied to the surface as described in the methods directly above. Solvents which may be used are as described in the methods directly above. Step (b) can be carried out using any known coating method which is suitable for the particular additional coating(s).

Following steps (a) and/or (b), a drying step may be required to remove the solvent. The drying may be, for example, via localized heating, or air drying, or drying under a gas stream (such as under argon).

If step (a) is carried out using chemical entities in thiol form, then the method can further comprise step (c) of converting at least a proportion of the thiol (—SH) groups in the chemical entity coating to S-nitrosothiol (—SNO) groups. Step (c) therefore comprises reacting the surface of step (a) or step (b) under conditions such that at least a proportion of the thiol groups are converted to S-nitrosothiol groups, such conditions being well known to a person having ordinary skill in the art.

In another embodiment, the chemical entities (in thiol form or in S-nitrosothiol form) are formed in situ, when forming a coating on surface. Put another way, one of components B and C is first coated onto a surface, and then the remaining component B or C is reacted with the coated surface, to form the chemical entity coating. An example of this method is set out in Example 31, where ePTFE film was first coated with PEI (component C), and then the coated films were reacted with adipoyl-1,6-dipenicillamine dithiolactone (component B), resulting the formation of a crosslinked PEI polymer (chemical entity).

Thus, in one embodiment is provided a method of forming a coating on a surface, wherein said method comprises the step of:
(a) contacting the surface with component B; and
(b) reacting the coated surface of step (a) with component C, such that component B reacts with component C, to form a chemical entity coating.

In another embodiment is provided a method of forming a coating on a surface, wherein said method comprises the step of:
(a) contacting the surface with component C; and
(b) reacting the coated surface of step (a) with component B, such that component C reacts with component B, to form a chemical entity coating.

Embodiments described above with respect to the chemical entity embodiments are equally applicable to the process/method embodiments.

It should also be noted that medical devices prepared according to the methods and processes described herein are also considered to form part of the present disclosure.

### Additional Clauses

1. A chemical entity formed by reaction of components B and C, wherein component B is a compound of Formula (I):

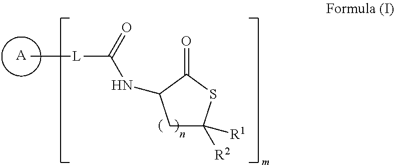

wherein,
A is a core moiety;
L is an optional linker;
R¹ and R² are independently selected from H and C₁₋₅ alkyl; or R¹ and R² together with the C atom to which they are attached combine to form a C₄₋₇ cycloalkyl ring or a 4-7 membered heterocyclic ring, both of which are optionally substituted on an available atom by one or more groups selected from C₁₋₂ alkyl and oxo;
n is 0 or 1; and
m is an integer of at least 1;
and,
component C is a moiety comprising one or more amine groups.
2. A surface having a coating comprising a chemical entity according to clause 1.
3. The chemical entity of clause 1, or the surface having a coating according to clause 2, wherein at least a proportion of the thiol (—SH) groups in the chemical entity have been converted to S-nitrosothiol (—SNO) groups.
4. A method of forming a coating on a surface, wherein said method comprises the step of: (a) contacting the surface with the chemical entity according to clause 1.
5. The method according to clause 4, further comprising step (b) of converting at least a proportion of the thiol (—SH) groups in the chemical entity to S-nitrosothiol (—SNO) groups.
6. The chemical entity according to clause 1, the surface having a coating according to clause 2 or clause 3, or the method according to clause 4 or clause 5, wherein core moiety A comprises one or more moieties independently selected from the group consisting of alkyl, spiroalkyl, aryl, heteroaryl, alkyl-aryl, a porphyrin, a polymer and a macrocycle, wherein alkyl, spiroalkyl, aryl, heteroaryl and alkyl-aryl are optionally substituted by one or more substituents (e.g. selected from the group consisting of alkyl, alkoxy, fluoroalkyl and fluoroalkoxy).
7. The chemical entity, surface having a coating, or method according to clause 6, wherein core moiety A comprises an alkyl group (e.g. C₁₋₂₀ alkyl) which is optionally substituted by one or more substituents (e.g. selected from the group consisting of alkyl, alkoxy, fluoroalkyl and fluoroalkoxy).
8. The chemical entity, surface having a coating, or method according to clause 7, wherein core moiety A comprises an alkyl group or is an alkyl group and m is 1 or 2, especially 2.
9. The chemical entity, surface having a coating, or method according to clause 8 wherein m is 2 and the core moiety A comprises or consists of a C₁₋₁₀ alkylene moiety, for example a C₁₋₆ alkylene moiety and especially a C₁₋₄ alkylene moiety such as —(CH₂)₄—, —(CH₂)₃—, —C(CH₃)₂—, —CH(CH₃)—, —CH₂—, —CH(CH₃)CH₂—, —CH₂CH(CH₃)— or —CH₂CH₂-.
10. The chemical entity, surface having a coating, or method according to clause 9 wherein A is —C(CH₃)₂— and m is 2.
11. The chemical entity, surface having a coating, or method according to clause 9 wherein A is —(CH₂)₄— and m is 2.
12. The chemical entity, surface having a coating, or method according to clause 6, wherein core moiety A comprises a spiroalkyl group (e.g. C₁₋₂₀ spiroalkyl) which is optionally substituted by one or more substituents (e.g. selected from the group consisting of alkyl, alkoxy, fluoroalkyl and fluoroalkoxy).
13. The chemical entity, surface having a coating, or method according to clause 6, wherein core moiety A comprises an aryl group (e.g. 5-20 membered aryl) which is optionally substituted by one or more substituents (e.g. selected from the group consisting of alkyl, alkoxy, fluoroalkyl and fluoroalkoxy).
14. The chemical entity, surface having a coating, or method according to clause 13, wherein the aryl group is phenyl, which is optionally substituted by one or more substituents selected from the group consisting of alkyl, alkoxy, fluoroalkyl and fluoroalkoxy.
15. The chemical entity according to clause 1, surface having a coating according to clause 2 or clause 3, or the method according to clause 4 or clause 5, wherein
m is 2 and core moiety A is:

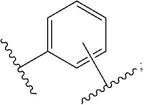

or
m is 3 and core moiety A is:

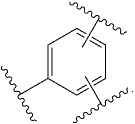

16. The chemical entity, surface having a coating or method according to clause 15 wherein: m is 2 and core moiety A is:

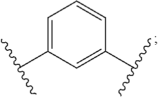 [SMILES: *c1cccc(*)c1]

or
m is 3 and core moiety A is:

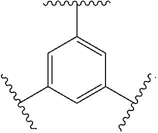 [SMILES: *c1cc(*)cc(*)c1]

17. The chemical entity, surface having a coating, or method according to clause 6, wherein core moiety A comprises an heteroaryl group (e.g. 5-10 membered heteroaryl) which is optionally substituted by one or more substituents (e.g. selected from the group consisting of alkyl, alkoxy, fluoroalkyl and fluoroalkoxy).
18. The chemical entity, surface having a coating, or method according to clause 6, wherein core moiety A comprises alkyl-aryl group (e.g. aryl substituted by alkyl (e.g. C₁₋₁₀ alkyl) or polyaryl linked by alkyl (e.g. C₁₋₅ alkyl)) which is optionally substituted by one or more substituents (e.g. selected from the group consisting of alkyl, alkoxy, fluoroalkyl and fluoroalkoxy).
19. The chemical entity, surface having a coating, or method according to clause 6, wherein core moiety A comprises a porphyrin e.g. tetra-4-aryl-meso.
20. The chemical entity, surface having a coating, or method according to clause 6, wherein core moiety A comprises a polymer, e.g. a polyamine compound (such as selected from the group consisting of polyethyleneimine, polyallylamine, polylysine, polyarginine and polyaminosilane), a dendrimer (such as a PAMAM dendrimer or a PPI-dendrimer) or a hyperbranched polymer.
21. The chemical entity, surface having a coating, or method according to clause 6, wherein core moiety A comprises a macrocycle, e.g. a cyclodextrin.
22. The chemical entity, surface having a coating, or method according to any one of clauses 1 to 21, wherein core moiety A comprises moieties that are labile.
23. The chemical entity, surface having a coating, or method according to clause 22, wherein core moiety A comprises moieties that are labile via hydrolysis or nucleophilic substitution, e.g. moieties containing one or more functional groups independently selected from the group consisting of an ester, an anhydride, an iminine, an imine, an acetal, a carbonate, a phosphazene, a phosphate ester, a urethane, a lactide and a lipolide.
24. The chemical entity, surface having a coating, or method according to clause 22, wherein core moiety A comprises moieties that are labile via reduction-oxidation e.g. moieties containing one or more functional groups independently selected from the group consisting of an oxide, a superoxide, ozone, an oxoacid, an oxyacid, a halite, a hypohalite and a hypohalous.
25. The chemical entity, surface having a coating, or method according to clause 22, wherein core moiety A comprises moieties that are labile via photolysis (e.g. by UV or visible light) e.g. moieties containing one or more functional groups independently selected from the group consisting of a benzoyl carbonyl and an azide.
26. The surface having a coating, or method according to any one of clauses 22 to 25, wherein the chemical entity is bioresorbable.
27. The chemical entity, surface having a coating, or method according to any one of clauses 1 to 26, wherein linker L comprises C₁₋₁₀ alkylene, a secondary amine or an amide.
28. The chemical entity, surface having a coating, or method according to any one of clauses 1 to 26, wherein linker L is absent.
29. The chemical entity, surface having a coating, or method according to any one of clauses 1 to 28, wherein n is 0.
30. The chemical entity, surface having a coating, or method according to any one of clauses 1 to 28, wherein n is 1.
31. The chemical entity, surface having a coating, or method according to any one of clauses 1 to 30, wherein R¹ and R² are independently selected from the group consisting of H and C₁₋₅ alkyl.
32. The chemical entity, surface having a coating, or method according to clause 31, wherein R¹ and R² are independently selected from the group consisting of CH₂CH₃ and CH₃; and in particular are both CH₃.
33. The chemical entity, surface having a coating, or method according to any one of clauses 1 to 30, wherein R¹ and R² together with the C atom to which they are attached combine to form a C₄₋₇ cycloalkyl ring or a 4-7 membered heterocyclic ring, both of which are optionally substituted on an available atom by one or more groups selected from C₁₋₂ alkyl and oxo.
34. The chemical entity, surface having a coating, or method according to clause 33, wherein R¹ and R² together with the C atom to which they are attached combine to form a cycloheptyl or cyclohexyl ring which is optionally substituted on an available atom by one or more groups selected from C₁₋₂ alkyl and oxo.
35. The chemical entity, surface having a coating, or method according to any one of clauses 1 to 14 or 17 to 34, wherein m is 1-5,000,000, 2-5,000,000, 1-1,000,000, 2-1,000,000, 1-500,000, 2-500,000, 1-400,000, 2-400,000, 1-200,000, 2-200,000, 1-100,000, 2-100,000, 1-50,000, 2-50,000, 1-20,000, 2-20,000, 1-10,000, 2-10,000, 1-5,000, 2-5,000, 1-2,000, 2-2,000, 1-1,000, 2-1,000, 1-500, 2-500, 1-100, 2-100, 1-50, 2-50, 1-20, 2-20, 1-10, 2-10, 1-5 or 2-5.
36. The chemical entity, surface having a coating, or method according to clause 35, wherein m is 2-500,000, 50-500,000, 100-500,000, 500-500,000, 1,000-500,000, 5,000-500,000, 1-200,000, 2-200,000, 50-200,000, 100-200,000, 500-200,000, 1,000-200,000, 5,000-200,000, 1-100,000, 2-100,000, 50-100,000, 100-100,000, 500-100,000, 1,000-100,000, 5,000-100,000, 1-50,000, 2-50,000, 50-50,000, 100-50,000, 500-50,000, 1,000-50,000, 5,000-50,000, 1-20,000, 2-20,000, 50-20,000, 100-20,000, 500-20,000, 1,000-20,000, 5,000-20,000, 1-5,000, 2-5,000, 50-5,000, 100-5,000, 500-5,000, 1,000-5,000, 1-20,000, 2-20,000, 50-20,000, 100-20,000, 500-20,000, 1,000-20,000, 5,000-20,000, 1-1,000, 2-1,000, 50-1,000, 100-1,000, or 500-1,000.
37. The chemical entity, surface having a coating, or method according to clause 36, wherein m is 1-1,000, 2-1,000, 50-1,000, 100-1,000, or 500-1,000.
38. The chemical entity, surface having a coating, or method according to clause 37, wherein m is 2, 3 or 4; and in particular is 2 or 3.
39. The compound, surface having a coating, or method according to clause 37, wherein m is 5-10 for example m is 5-8.
40. The chemical entity, surface having a coating, or method according to clause 35 or clause 36, wherein m is 5,000-100,000 for example m is 5,000-50,000 or 5,000-20,000.
41. The chemical entity according to clause 1, surface having a coating according to clause 2 or clause 3, or the method according to clause 4 or clause 5 wherein
component B is of Formula (Ia):

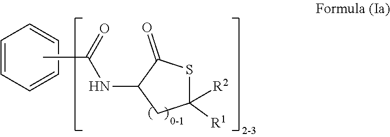

wherein,
R¹ and R² are independently selected from H and C₁₋₅ alkyl, e.g. selected from CH₃ and CH₂CH₃ (and in particular are both CH₃); or R¹ and R² together with the C atom to which they are attached combine to form a cycloheptyl or cyclohexyl ring which is optionally substituted on an available atom by one or more groups selected from C₁₋₂ alkyl and oxo.
42. The chemical entity, surface having a coating, or method according to clause 41, wherein,
component B is:

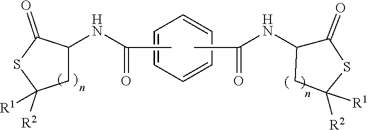

wherein n is 0 or 1, and R¹ and R² are independently selected from H and C₁₋₅ alkyl;
wherein, when n is 0, R¹ and R² are suitably both C₁₋₅ alkyl, in particular methyl or ethyl, e.g. are both methyl; and when n is 1, R¹ and R² are suitably both H.
43. The chemical entity, surface having a coating, or method according to clause 41, wherein,
component B is:

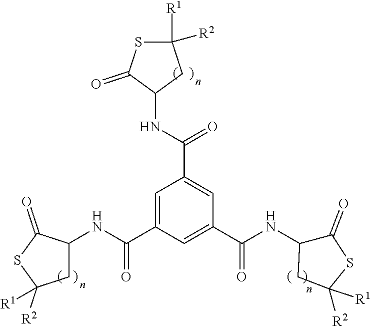

wherein n is 0 or 1, and R¹ and R² are independently selected from H and C₁₋₅ alkyl;
wherein, when n is 0, R¹ and R² are suitably both C₁₋₅ alkyl, in particular methyl or ethyl, e.g. are both methyl; and when n is 1, R¹ and R² are suitably both H.
44. The chemical entity, surface having a coating, or method according to clause 1, surface having a coating according to clause 2 or clause 3, or the method according to clause 4 or clause 5 wherein component B is:

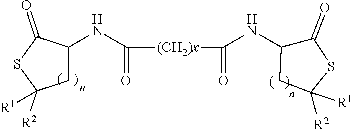

wherein n is 0 or 1, x is 1-10, for example 1-6 and R¹ and R² are independently selected from H and C₁₋₅ alkyl;
wherein, when n is 0, R¹ and R² are suitably both C₁₋₅ alkyl, in particular methyl or ethyl, e.g. are both methyl; and when n is 1, R¹ and R² are suitably both H.
45. The chemical entity, surface having a coating, or method according to clause 1, surface having a coating according to clause 2 or clause 3, or the method according to clause 4 or clause wherein component B is of Formula (Iaa):

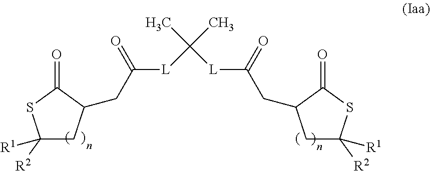

wherein L, n, R¹ and R² are as defined in clause 1.
46. The chemical entity, surface having a coating, or method according to clause 45 wherein L is absent and component B is of Formula (Iab):

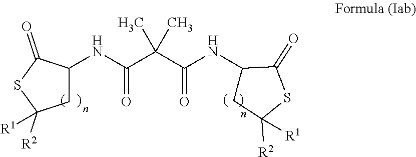

wherein n, R¹ and R² are as defined in clause 1.
47. The chemical entity, surface having a coating, or method according to clause 46 wherein L is absent, n is 0 and component B is of Formula (Iac):

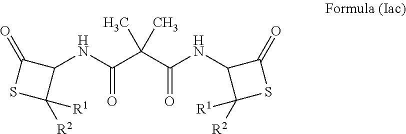

wherein R¹ and R² are as defined in clause 1.
48. The chemical entity, surface having a coating, or method according to any one of clauses to 47 wherein R¹ and R² are suitably independently selected from H and C₁₋₅ alkyl, suitably H, methyl or ethyl and more suitably H or methyl.
49. The chemical entity, surface having a coating, or method according to any one of clauses 1 to 48, wherein component B is a D-stereoisomer.
50. The chemical entity, surface having a coating, or method according to any one of clauses 1 to 48, wherein component B is an L-stereoisomer.
51. The chemical entity, surface having a coating, or method according to any one of clauses 1 to 48, wherein component B is a mixture (such as a racemic mixture) of D- and L-stereoisomers.
52. The chemical entity, surface having a coating, or method according to any one of clauses 1 to 51 wherein component C is a single compound or a mixture of compounds.
53. The chemical entity, surface having a coating, or method according to clause 52, wherein component C is a single monomeric compound (i.e. a single compound of well defined composition).
54. The chemical entity, surface having a coating, or method according to clause 52, wherein component C is a polymer.
55. The chemical entity, surface having a coating, or method according to clause 52, wherein component C is a mixture of two or more compounds, especially two compounds, selected from monomeric compounds and polymers.
56. The chemical entity, surface having a coating, or method according to clause 55, wherein component C is a mixture of two or more monomeric compounds, wherein, at least one or all of the two or more monomeric compounds comprise two or more amine groups e.g. two amine groups.
57. The chemical entity, surface having a coating, or method according to clause 55, wherein component C is a mixture of two or more polymers.
58. The chemical entity, surface having a coating, or method according to any one of clauses 54 to 57 wherein the polymer is formed from a single monomer and is, for example, polyethylene imine, polylysine, polypropylene oxide or polyethylene oxide.
59. The chemical entity, surface having a coating, or method according to any one of clauses 54 to 57 wherein the polymer is a copolymer formed from two or more monomers, for example two monomers and, for example, is a polymer formed from units of propylene oxide and ethylene oxide.
60. The chemical entity, surface having a coating, or method according to any one of clauses 1 to 59, wherein the amine groups of component C are independently primary or secondary amine groups, wherein, secondary amine groups may be present in a cyclic amine such as morpholine, piperidine or piperazine.
61. The chemical entity, surface having a coating, or method according to any one of clauses 1-60, wherein component C comprises a single amine group.
62. The chemical entity, surface having a coating, or method according to clause 61, wherein component C is selected from the group consisting of aniline, benzylamine, morpholine, diethylamine, dibutylamine, 1-ethylpropylamine, 2-aminopentane, piperidine, 4-methylpiperidine, pyrrolidine, t-butylamine, t-amylamine, propylamine, butylamine, amylamine, isopentylamine, hexylamine, heptylamine, octylamine, nonylamine, decylamine, dodecylamine, hexadecylamine, octadecylamine, 4-methylaniline, 2-fluorobenzylamine, 3-fluorobenzylamine, 4-fluorobenzylamine, 3-(trifluoromethyl)benzylamine, 4-(trifluoromethyl)benzylamine and 3,5-bis(trifluoromethyl)benzylamine, especially aniline, benzylamine and morpholine.
63. The chemical entity, surface having a coating, or method according to any one of clauses 1 to 60, wherein component C comprises two or more amine groups, wherein said amine groups are independently primary or secondary amine groups.
64. The chemical entity, surface having a coating, or method according to clause 63, wherein component C comprises a polyamine compound, for example, component C comprises one or more moieties independently selected from the group consisting of polyethyleneimine, polyallylamine, polylysine, polyarginine and polyaminosilane.
65. The chemical entity, surface having a coating, or method according to clause 64, wherein component C comprises polyethyleneimine e.g. branched polyethyleneimine having a molecular weight of 10-1,000 kDa e.g. 10-50 kDa or 50-100 kDa.
66. The chemical entity, surface having a coating, or method according to any one of clauses 1 to 65, wherein component C comprises a polyamine compound comprising a dendrimer or a hyperbranched polymer.
67. The chemical entity, surface having a coating, or method according to any one of clauses 1 to 63, wherein component C is a straight chain polymer comprising two amine groups, suitably primary amine groups or secondary amine groups, one at each terminus of the polymer chain.
68. The chemical entity, surface having a coating, or method according to any one of clauses 1 to 67, wherein component C is a compound of Formula (X):

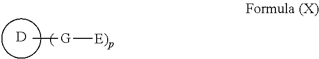

wherein,
D is a core moiety;
G is an optional linker;
E is NH₂; and
p is an integer of at least 1.
69. The chemical entity, the surface having a coating, or method according to clause 68, wherein core moiety D comprises one or more moieties independently selected from the group consisting of alkyl, spiroalkyl, aryl, heteroaryl, alkyl-aryl, a porphyrin, a polymer and a macrocycle, wherein alkyl, spiroalkyl, aryl, heteroaryl and alkyl-aryl are optionally substituted by one or more substituents (e.g. selected from the group consisting of alkyl, alkoxy, fluoroalkyl and fluoroalkoxy).
70. The chemical entity, the surface having a coating, or method according to clause 69, wherein core moiety D comprises one or more C₂₋₄ alkylene oxide units, for example ethylene oxide units or propylene oxide units (i.e. D is a polyoxyC₂₋₄ alkylene).
71. The chemical entity, the surface having a coating, or method according to clause 70, wherein component C is a single compound of Formula (X) wherein the average number of alkylene oxide units in the core moiety D is from about 2 to about 20, for example about 2 to 10, 3 to 7 or 3.5 to 6.
72. The chemical entity, the surface having a coating, or method according to clause 70, wherein component C is a mixture of a first compound of Formula (X) and a second compound of Formula (X).
73. The chemical entity, the surface having a coating, or method according to clause 72, wherein Component C is a mixture of a first compound of Formula (X) wherein D is a polymer which comprises an average number of alkylene oxide units from about 2 to about 20, for example about 2 to 10, 3 to 7 or 3.5 to 6, and a second compound of Formula (X) wherein D is a polymer which comprises an average number of alkylene oxide units from about 11 to about 100, for example about 20 to about 60 or 30 to 50.
74. The chemical entity, the surface having a coating, or method according to any one of clauses 68 to 73, wherein the linker G is absent and p is 2.
75. The chemical entity, the surface having a coating, or method according to clause 68, wherein core moiety D is a polyalkylsiloxane moiety substituted with one or more primary and/or secondary amine groups, especially one or more primary amine groups.
76. The chemical entity, surface having a coating, or method according to clause 69, wherein core moiety D comprises an alkyl group (e.g. C₁₋₂₀ alkyl) which is optionally substituted by one or more substituents (e.g. selected from the group consisting of alkyl, alkoxy, fluoroalkyl and fluoroalkoxy).
77. The chemical entity, surface having a coating, or method according to clause 69, wherein core moiety D comprises an aryl group (e.g. 5-20 membered aryl) which is optionally substituted by one or more substituents (e.g. selected from the group consisting of alkyl, alkoxy, fluoroalkyl and fluoroalkoxy).
78. The chemical entity, surface having a coating, or method according to clause 77, wherein the aryl group is phenyl, which is optionally substituted by one or more substituents selected from the group consisting of alkyl, alkoxy, fluoroalkyl and fluoroalkoxy.
79. The chemical entity, surface having a coating, or method according to clause 69, wherein core moiety D comprises a spiroalkyl group (e.g. C₁-20 spiroalkyl) which is optionally substituted by one or more substituents (e.g. selected from the group consisting of alkyl, alkoxy, fluoroalkyl and fluoroalkoxy); or
wherein core moiety D comprises an heteroaryl group (e.g. 5-10 membered heteroaryl) which is optionally substituted by one or more substituents (e.g. selected from the group consisting of alkyl, alkoxy, fluoroalkyl and fluoroalkoxy); or
wherein core moiety D comprises alkyl-aryl group (e.g. aryl substituted by alkyl (e.g. C₁₋₁₀ alkyl) or polyaryl linked by alkyl (e.g. C₁₋₅ alkyl)) which is optionally substituted by one or more substituents (e.g. selected from the group consisting of alkyl, alkoxy, fluoroalkyl and fluoroalkoxy); or
wherein core moiety D comprises a porphyrin e.g. tetra-4-aryl-meso; or
wherein core moiety D comprises a macrocycle, e.g. a cyclodextrin.
80. The chemical entity, surface having a coating, or method according to clause 69, wherein core moiety D comprises a polymer e.g. a polyamine compound (such as selected from the group consisting of polyethyleneimine, polyallylamine, polylysine, polyarginine and polyaminosilane), a dendrimer (such as a PAMAM dendrimer or a PPI-dendrimer) or a hyperbranched polymer.
81. The chemical entity, surface having a coating, or method according to any one of clauses 68 to 80, wherein core moiety D comprise moieties which are labile
82. The chemical entity, surface having a coating, or method according to clause 81, wherein core moiety D comprises moieties that are labile via hydrolysis or nucleophilic substitution, e.g. moieties containing one or more functional groups independently selected from the group consisting of an ester, an anhydride, an iminine, an imine, an acetal, a carbonate, a phosphazene, a phosphate ester, a urethane, a lactide and a lipolide; or that are labile via reduction-oxidation e.g. moieties containing one or more functional groups independently selected from the group consisting of an oxide, a superoxide, ozone, an oxoacid, an oxyacid, a halite, a hypohalite and a hypohalous; or
that are labile via photolysis (e.g. by UV or visible light) e.g. moieties containing one or more functional groups independently selected from the group consisting of a benzoyl carbonyl and an azide.
83. The chemical entity, surface having a coating, or method according to any one of clauses 68 to 82, wherein core moiety D comprises secondary amine groups.
84. The chemical entity, surface having a coating, or method according to any one of clauses 68 to 83, wherein core moiety D comprises C₁₋₁₀ alkylene, aryl, aryl-(CH₂)₁₋₁₀-aryl or —[CH₂CH₂NH]_{1-20,000}.
85. The chemical entity, surface having a coating, or method according to any one of clauses 68 to 84, wherein G comprises the moiety —(CH₂CH₂NH)₁₋₅—CH₂CH₂—.
86. The chemical entity, surface having a coating, or method according to any one of clauses 68 to 84, wherein G is absent.
87. The chemical entity, surface having a coating, or method according to any one of clauses 68 to 86, wherein E is N(R^{z})H, where each R^{z} is H or C₁₋₆ alkyl, for example, each R^{z} is independently H or C₁₋₃ alkyl, such as H, methyl or ethyl.
88. The chemical entity, surface having a coating, or method according to clause 87, wherein:
each E is NH₂; or
each E is NH(C₁₋₆ alkyl), for example NH(CH₃); or
p>1 and one or more E is NH₂ and one or more E is NH(C₁₋₆ alkyl); or
p is 2, both E groups are NH₂ groups or both E groups are NH(C₁₋₆ alkyl), such as NH(CH₃) or one E group is NH₂ and the other E group is NH(C₁₋₆ alkyl), such as NH(CH₃).
89. The chemical entity, surface having a coating, or method according to any one of clauses 68 to 88, wherein p is 1-5,000,000, 2-5,000,000, 1-1,000,000, 2-1,000,000, 1-500,000, 2-500,000, 1-400,000, 2-400,000, 1-200,000, 2-200,000, 1-100,000, 2-100,000, 1-50,000, 2-50,000, 1-20,000, 2-20,000, 1-10,000, 2-10,000, 1-5,000, 2-5,000, 1-2,000, 2-2,000, 1-1,000, 2-1,000, 1-500, 2-500, 1-100, 2-100, 1-50, 2-50, 1-20, 2-20, 1-10, 2-10, 1-5 or 2-5.
90. The chemical entity, surface having a coating, or method according to clause 89, wherein p is 1-100,000, 2-100,000, 2-50,000, 2-20, 2-12, 2-5, 2-4 or 2-3.
91. The chemical entity, surface having a coating, or method according to any one of clauses 1 to 90, wherein component C is of Formula (Xx):

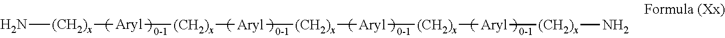

wherein each x is independently 0-10.
92. The chemical entity, surface having a coating, or method according to clause 91, wherein component C is of Formula (Xxx):

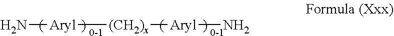

wherein x is 0-10; or
wherein component C is of Formula (Xxy):

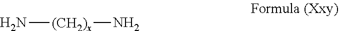

wherein x is 1-10, e.g. 3-8.
93. The chemical entity, surface having a coating, or method according to clause 92, wherein: component C is H₂N—(CH₂)₆—NH₂ or H₂N—(CH₂)₄—NH₂; or component C is of Formula (Xxz):

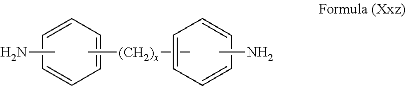

wherein x is 0-10, such as 1.
94. The chemical entity, surface having a coating, or method according to any one of clauses 1 to 90, wherein component C is of Formula (Xu):

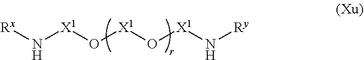

wherein X¹ is a straight or branched C₂₋₄ alkylene group, r is a number representing the average number of alkylene oxide units per molecule and each of R^{x} and R^{y} is H or C₁₋₆ alkyl, such as H or C₁₋₃ alkyl, for example H, methyl or ethyl, especially H or methyl.
95. The chemical entity, surface having a coating, or method according to claim 94, wherein the compound of Formula (Xu) is a compound of Formula (Xva):

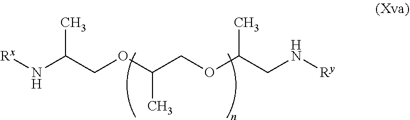

which is based on a core, D, comprising propylene oxide units; or
a compound of Formula (Xvb)

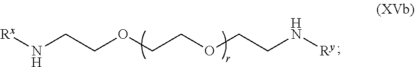

which is based on a core, D, comprising ethylene oxide units, wherein r, R^{x} and R^{y} are as defined in clause 94, for example r is 2 to 10.
96. The chemical entity, surface having a coating, or method according to clause 94, wherein in the compound of Formula (Xu) not all X¹ groups are the same, for example some X¹ groups are ethylene oxide units and other X¹ groups are propylene oxide units, and r is from about 11 to about 100, for example 20 to 60 or 30 to 50.
97. The chemical entity, surface having a coating, or method according to any one of clauses 1 to 90, wherein component C is an amine functionalized polyalkylsiloxane of Formula (Xt):

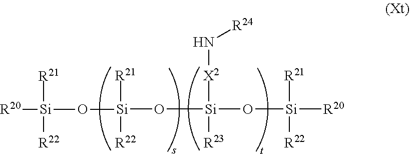

wherein each of R²⁰, R²¹, R²² and R²³ is independently C₁₋₆ alkyl, more suitably C₁₋₄ alkyl, R²⁴ is H or C₁₋₄ alkyl, especially H, X² is a straight or branched C₁₋₆ alkylene group, more suitably a straight or branched C₁₋₄ alkylene group, especially (CH₂)₂₋₄ and each of s and t is independently an integer of from about 10 to 400, more suitably from about 50 to 250.
98. The chemical entity, surface having a coating, or method according to any one of clauses 1 or 3 to 97, wherein the chemical entity comprises:

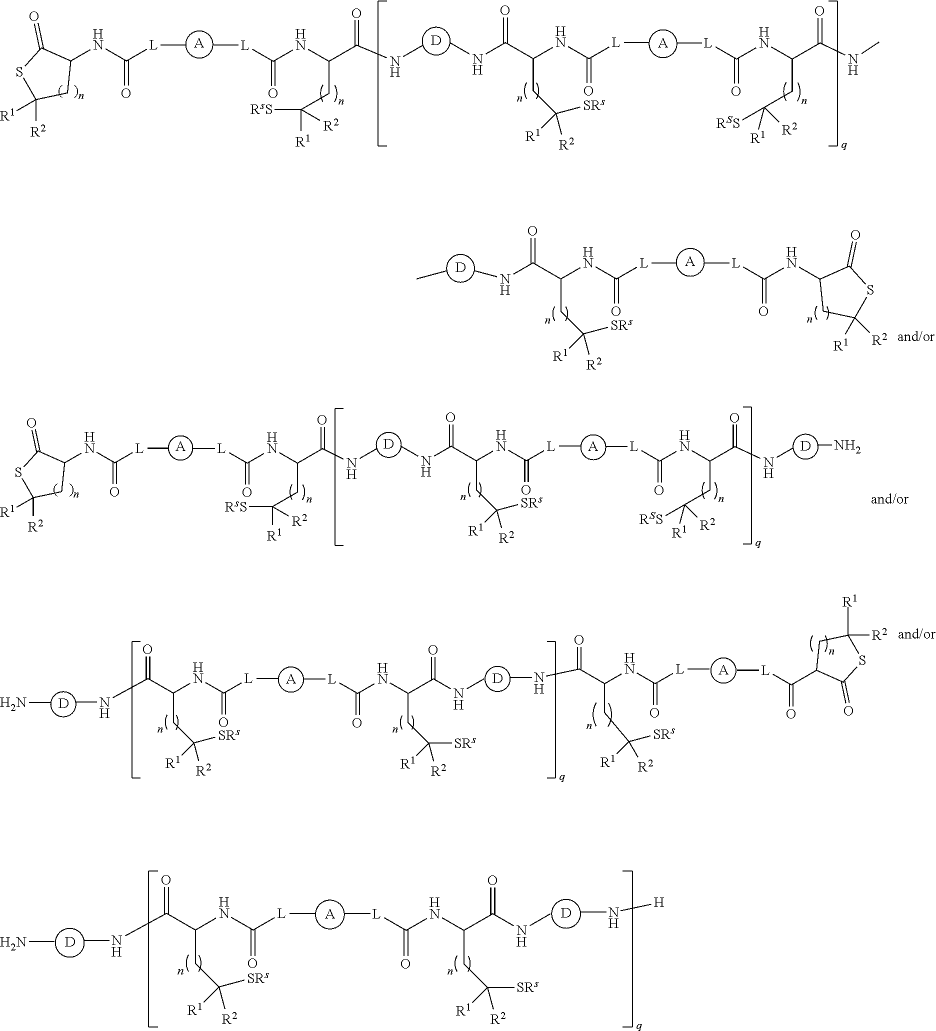

wherein,
A, L, n, R¹, R², and D are as defined in any one of clauses i to 97;
R⁵ is H or N═O; and
q is 10 to 1000, for example 15 to 500, 20 to 250 or 25 to 180.
99. The chemical entity, surface having a coating, or method according to clause 98, wherein the chemical entity comprises:

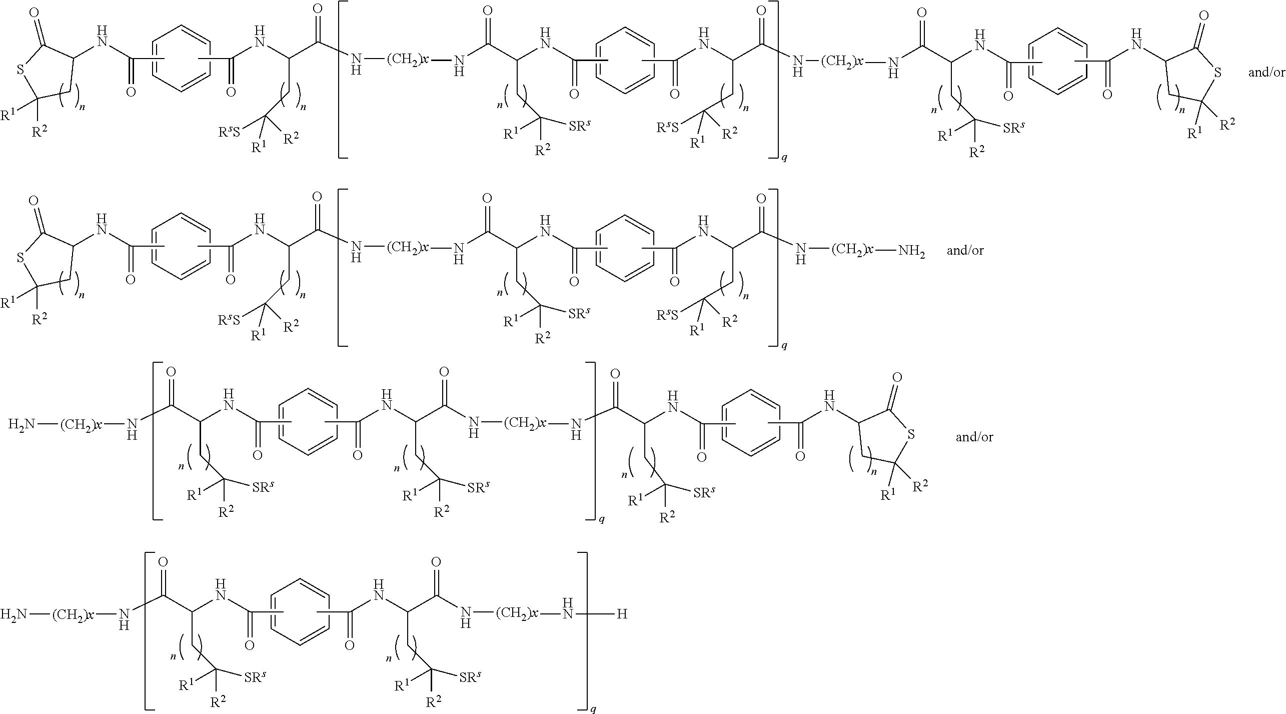

wherein n is as defined in any one of clauses 1 to 98;
x is 1-10;
R^{s} is H or N═O; and
q is 10 to 1000, for example 15 to 500, 20 to 250 or 25 to 180.
100. The chemical entity, surface having a coating, or method according to clause 98 wherein the chemical entity comprises:

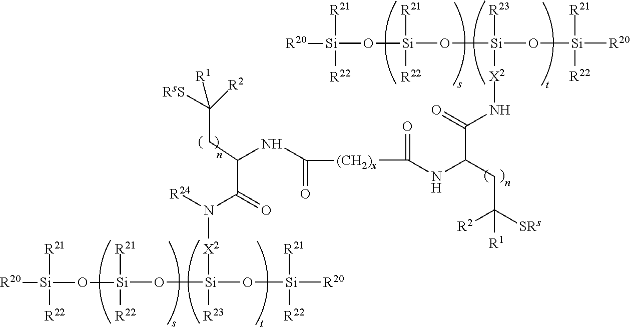

wherein R¹, R², R²⁰, R²¹, R²², R²³, n, X², s and t are as defined herein;
R^{s} is H or N═O; and
x is 1-10, suitably 1-6 and more suitably 1-4.

The polymer typically has a weight average molecular weight of about 50,000 to 200,000, for example about 100,000.
101. The chemical entity, surface having a coating, or method according to clause 98, wherein the chemical entity comprises:

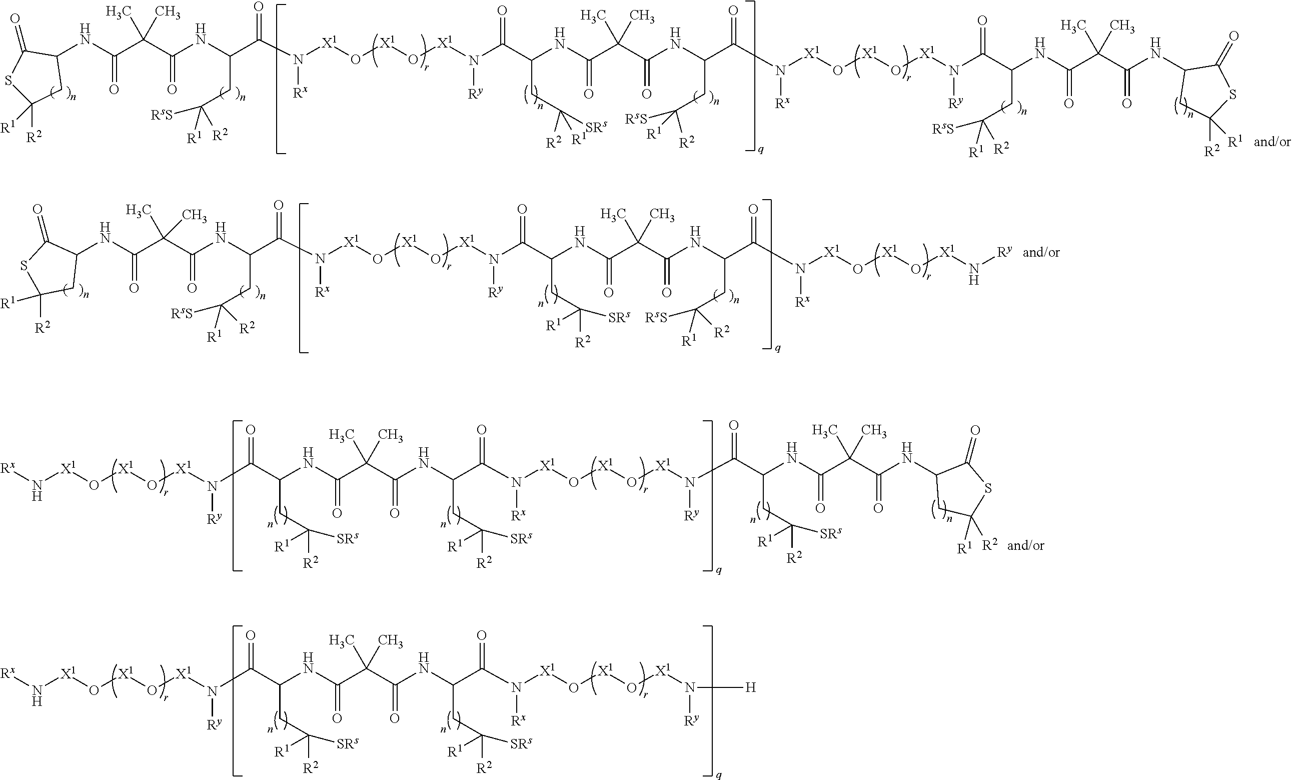

wherein n, X¹ and r are as defined in any one of clauses 1 to 99;
R^{s} is H or N═O;
each of R^{x} and RY is H or C₁₋₆alkyl and
q is 10 to 1000, for example 15 to 500, 20 to 250 or 25 to 180.
102. The chemical entity, surface having a coating, or method according to clause 98, wherein the chemical entity comprises:

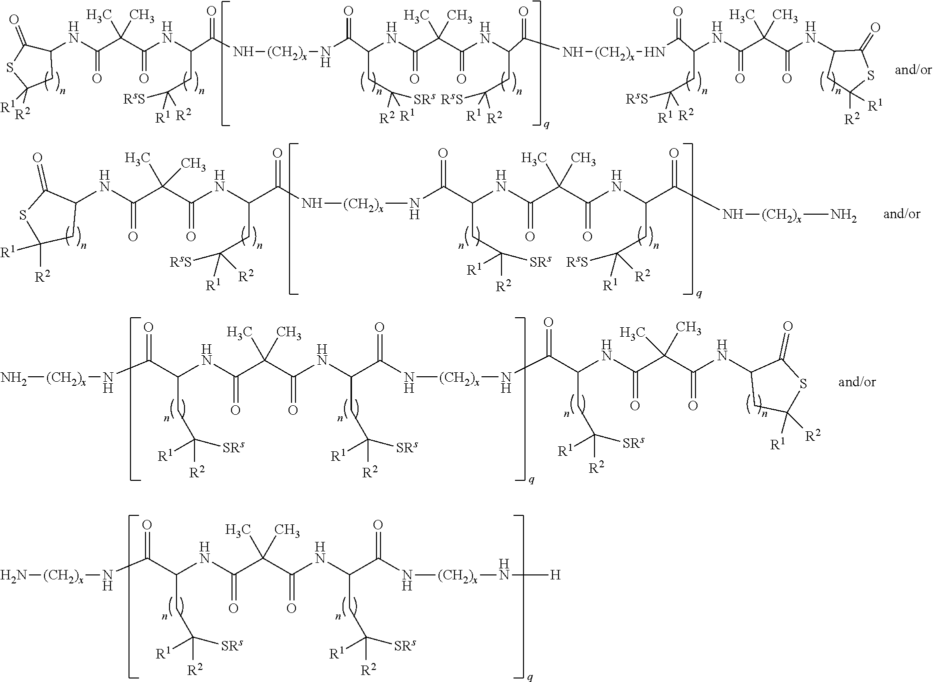

x is 1-10, for example 2-6;
R^{s} is H or N═O; and
q is 10 to 1000, for example 15 to 500, 20 to 250 or 25 to 180. 103. The chemical entity, surface having a coating, or method according to any one of clauses 1 to 102, wherein the chemical entity has 20-100% of thiol (—SH) groups, such as 30-100%, 40-100%, 50-100% or 60-100% of free SH groups, relative to the molar % of component B.
104. The chemical entity, surface having a coating, or method according to any one of clauses 3 to 103, wherein the chemical entity has 20-100% of S-nitrosothiol (—SNO) groups, such as 30-100%, 40-100%, 50-100% or 60-100% of free SH groups, relative to the molar % of component B.
105. A surface with a coating obtainable according to the method of any one of clauses 3 to 104.
106. A surface with a coating according to any one of clauses 3 to 105, wherein the surface comprises expanded polytetrafluoroethylene (ePTFE) or expanded polyethylene (ePE), in particular ePTFE.
107. A surface having a coating, or method according to any one of clauses 4 to 106, wherein the surface has one or more additional coatings, for example, one or more additional coatings are selected from the group consisting of a synthetic or naturally occurring organic or inorganic polymer or material, such as polyolefins, polyesters, polyurethanes, polyamides, polyether block amides, polyimides, polycarbonates, polyphenylene sulfides, polyphenylene oxides, polyethers, silicones, polycarbonates, polyhydroxyethylmethacrylate, polyvinyl pyrrolidone, polyvinyl alcohol, rubber, silicone rubber, polyhydroxyacids, polyallylamine, polyallylalcohol, polyacrylamide, polyacrylic acid, styrenic polymers, fluoropolymers, (e.g. expanded polytetrafluoroethylene (ePTFE), polytetrafluoroethylene (PTFE), fluorinated ethylene-propylene (FEP), perfluorocarbon copolymers (e.g. tetrafluoroethylene perfluoroalkylvinyl ether (TFE/PAVE) copolymers, copolymers of tetrafluoroethylene (TFE) and perfluoromethyl vinyl ether (PMVE)), copolymers of TFE with functional monomers that comprise acetate, alcohol, amine, amide and/or sulfonate, VDF-HFP), bioresorbables, such as poly(D,L-lactide), polyglycolids and copolymers thereof, non-woven, bioabsorbable web materials comprising a tri-block copolymer such as poly(glycolide-co-trimethylene carbonate) tri-block copolymer (PGA:TMC), nylon 12, nylon 11, nylon 9, nylon 6/9 and nylon 6/6, polyetheresteramide, polyethylene terephthalate and polybutylene terephthalate, polyester ethers, polyester elastomer copolymers, block copolymer elastomers such as those copolymers having styrene end blocks, and midblocks formed from butadiene, isoprene, ethylene/butylene, ethylene/propene, styrenic block copolymers including acrylonitrile-styrene and acrylonitrile-butadiene-styrene block copolymers, polystyrenes, poly(methyl)methacrylates, polyacrylonitriles, poly(vinylacetates), poly(vinyl alcohols), chlorine-containing polymers such as poly(vinyl) chloride, polyoxymethylenes, polycarbonates, polyamides, polyimides, polyurethanes, phenolics, amino-epoxy resins, polyesters, silicones, cellulose-based plastics, rubber-like plastics, expanded polyethylene, polyvinylchloride, polyurethane, silicone, polyethylene, polypropylene, polyurethane, polyglycolic acid, polyesters, polyamides, elastomers and their mixtures, blends and copolymers or derivatives.
108. The surface having a coating, or method according to clause 107, wherein at least one of the one or more additional coatings comprises a therapeutic agent.
109. The surface having a coating, or method according to clause 108, wherein the therapeutic agent is selected from the group consisting of an antithrombogenic agent, an anti-calcification agent, a hemostatic agent, an anti-angiogenic agent, an angiogenic agents, an anti-microbial agent, an anti-proliferative agent, a proliferative agent and an anti-inflammatory agent, or a combination thereof.
110. The surface having a coating, or method according to clause 109, wherein the therapeutic agent is an antithrombogenic agent, for example heparin e.g. an additional coating is the Carmeda® BioActive Surface (CBAS®).
111. The surface having a coating, or method according to any one of clauses 106 to 110, wherein at least one of the one or more additional coatings is a subsequent coating.
112. The surface having a coating, or method according to any one of the clauses 3 to 110, wherein the surface is the surface of a medical device.
113. The surface having a coating, or method according to clause 112, wherein the medical device comprises expanded polytetrafluoroethylene (ePTFE) or expanded polyethylene (ePE), in particular ePTFE.
114. The surface having a coating, or method according to clause 112 or clause 113, wherein the medical device is selected from the group consisting of stents including bifurcated stents, balloon expandable stents and self-expanding stents, stent-grafts including bifurcated stent-grafts, grafts including vascular grafts, bifurcated grafts and early cannulation grafts, dialators, vascular occluders, embolic filters, vascular filters, embolectomy devices, catheters including microcatheters, central venous catheters, peripheral intravenous catheters, indwelling catheters and hemodialysis catheters, artificial blood vessels, sheaths including retractable sheaths, blood indwelling monitoring devices, artificial heart valves, pacemaker electrodes, guidewires, cardiac leads, cardiopulmonary bypass circuits, cannulae, plugs, drug delivery devices, balloons, tissue patch devices, blood pumps, artificial pancreas devices, cell encapsulation devices, implantable sensor devices, embolic particles, drug releasing particles and liquid embolics.
115. A medical device comprising a surface having a coating according to any one of clauses 3 to 114.
116. The chemical entity according to any one of clauses 3 to 104, for use in the preventing or treating a condition which benefits from the delivery of nitric oxide.
117. The chemical entity for use according to clause 116, for use in opening blood vessels and/or improving oxygen levels.
118. The chemical entity for use according to clause 116, for use in the preventing or treating an atherosclerotic lesion or a hypervascular lesion.
119. The chemical entity for use according to clause 116, for use in the preventing or treating angiogenesis (including vasculogenesis).
120. The chemical entity for use according to clause 116, for use in promoting endothelialization, in particular of grafts/stent-grafts/stents.
121. The chemical entity for use according to clause 116, for use in preventing or treating infection, in particular bacterial infection.
122. The chemical entity for use according to clause 116, for maturing an arteriovenous fistula.
123. The chemical entity for use according to clause 116, for preventing or treating a skin ulcer, e.g. a foot ulcer.
124. The chemical entity for use according to clause 116, for acceleration of healing from the inflammation cycle.
125. A method for preventing or treating a condition which benefits from the delivery of nitric oxide, where a medical device according to clause 115 is used.
126. A method for opening blood vessels and/or improving oxygen levels, where a medical device according to clause 115 is used.
127. A method for preventing or treating an atherosclerotic lesion or a hypervascular lesion, where a medical device according to clause 115 is used.
128. A method for preventing or treating angiogenesis (including vasculogenesis), where a medical device according to clause 115 is used.
129. A method for promoting endothelialization, in particular of grafts/stent-grafts/stents, where a medical device according to clause 115 is used.
130. A method for preventing or treating infection, in particular bacterial infection, where a medical device according to clause 115 is used.
131. A method for maturing an arteriovenous fistula, where a medical device according to clause 115 is used.
132. A method for preventing or treating a skin ulcer, e.g. a foot ulcer, where a medical device according to clause 115 is used.
133. A method for acceleration of healing from the inflammation cycle, where a medical device according to clause 115 is used.
134. A surface having a coating, use or a method according to any one of clauses 4 to 114, wherein the coating is an antibacterial coating.
135. A surface having a coating according to any one of clauses 4 to 114, wherein the coating is stable to sterilization, in particular ethylene oxide sterilization.
136. A compound of formula (I):

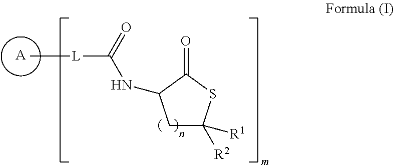

wherein,
A is a core moiety;
L is an optional linker;
R¹ and R² are independently selected from H and C₁₋₅ alkyl; or R¹ and R² together with the C atom to which they are attached combine to form a C₄₋₇ cycloalkyl ring or a 4-7 membered heterocyclic ring, both of which are optionally substituted on an available atom by one or more groups selected from C₁₋₂ alkyl and oxo;
n is 0 or 1; and
m is an integer of at least 1.
137. The compound of formula (I) according to clause 136, wherein core moiety A comprises one or more moieties independently selected from the group consisting of alkyl, spiroalkyl, aryl, heteroaryl, alkyl-aryl, a porphyrin, a polymer and a macrocycle, wherein alkyl, spiroalkyl, aryl, heteroaryl and alkyl-aryl are optionally substituted by one or more substituents (e.g. selected from the group consisting of alkyl, alkoxy, fluoroalkyl and fluoroalkoxy).
138. The compound of formula (I) according to clause 137, wherein core moiety A comprises an alkyl group (e.g. C₁₋₂₀ alkyl) which is optionally substituted by one or more substituents (e.g. selected from the group consisting of alkyl, alkoxy, fluoroalkyl and fluoroalkoxy).
139. The compound of formula (I) according to clause 137, wherein core moiety A comprises an alkyl group or is an alkyl group and m is 1 or 2, especially 2.
140. The compound of formula (I) according to clause 139, wherein core moiety A comprises or consists of a C₁₋₁₀ alkylene moiety, for example a C₁₋₆ alkylene moiety and especially a C₁₋₃ alkylene moiety such as —C(CH₃)₂—, —CH(CH₃)—, —CH₂—, —CH(CH₃)CH₂—, —CH₂CH(CH₃)— or —CH₂CH₂—.
141. The compound of formula (I) according to clause 140 wherein core moiety A is —C(CH₃)₂— and m is 2.
142. The compound of formula (I) according to clause 137, wherein core moiety A comprises a spiroalkyl group (e.g. C₁₋₂₀ spiroalkyl) which is optionally substituted by one or more substituents (e.g. selected from the group consisting of alkyl, alkoxy, fluoroalkyl and fluoroalkoxy).
143. The compound of formula (I) according to clause 137, wherein core moiety A comprises an aryl group (e.g. 5-10 membered aryl) which is optionally substituted by one or more substituents (e.g. selected from the group consisting of alkyl, alkoxy, fluoroalkyl and fluoroalkoxy).
144. The compound of formula (I) according to clause 143, wherein the aryl group is phenyl, which is optionally substituted by one or more substituents selected from the group consisting of alkyl, alkoxy, fluoroalkyl and fluoroalkoxy.
145. The compound of formula (I) according to clause 137, wherein m is 2 and core moiety A is:

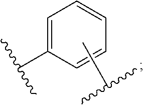

or
m is 3 and core moiety A is:

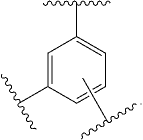

146. The compound of formula (I) according to clause 145, wherein
m is 2 and core moiety A is:

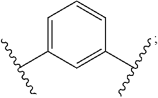 [SMILES: *c1cccc(*)c1]

or
m is 3 and core moiety A is:

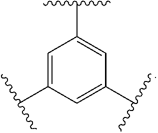 [SMILES: *c1cc(*)cc(*)c1]

147. The compound of formula (I) according to clause 137, wherein core moiety A comprises an heteroaryl group (e.g. 5-10 membered heteroaryl) which is optionally substituted by one or more substituents (e.g. selected from the group consisting of alkyl, alkoxy, fluoroalkyl and fluoroalkoxy).
148. The compound of formula (I) according to clause 137, wherein core moiety A comprises an alkyl-aryl group (e.g. aryl substituted by alkyl (e.g. C₁₋₁₀ alkyl) or polyaryl linked by alkyl (e.g. C₁₋₅ alkyl)) which is optionally substituted by one or more substituents (e.g. selected from the group consisting of alkyl, alkoxy, fluoroalkyl and fluoroalkoxy).
149. The compound of formula (I) according to clause 137, wherein core moiety A comprises a porphyrin e.g. tetra-4-aryl-meso.
150. The compound of formula (I) according to clause 137, wherein core moiety A comprises a polymer, e.g. a polyamine compound (such as selected from the group consisting of polyethyleneimine, polyallylamine, polylysine, polyarginine and polyaminosilane), a dendrimer (such as a PAMAM dendrimer or a PPI-dendrimer) or a hyperbranched polymer.
151. The compound of formula (I) according to clause 137, wherein core moiety A comprises a macrocycle, e.g. a cyclodextrin.
152. The compound of formula (I) according to any one of clauses 136 to 151, wherein core moiety A comprise moieties that are labile.
153. The compound of formula (I) according to clause 152, wherein core moiety A comprises moieties that are labile via hydrolysis or nucleophilic substitution, e.g. moieties containing one or more functional groups independently selected from the group consisting of an ester, an anhydride, an iminine, an imine, an acetal, a carbonate, a phosphazene, a phosphate ester, a urethane, a lactide and a lipolide.
154. The compound of formula (I) according to clause 152, wherein core moiety A comprises moieties which are labile via reduction-oxidation e.g. moieties containing one or more functional groups independently selected from the group consisting of an oxide, a superoxide, ozone, an oxoacid, an oxyacid, a halite, a hypohalite and a hypohalous.
155. The compound of formula (I) according to clause 152, wherein core moiety A comprises moieties which are labile via photolysis (e.g. by UV or visible light) e.g. moieties containing one or more functional groups independently selected from the group consisting of a benzoyl carbonyl and an azide.
156. The compound of formula (I) according to any one of clauses 136 to 155, wherein linker L comprises C₁₋₁₀ alkylene, a secondary amine or an amide.
157. The compound of formula (I) according to any one of clauses 136 to 155, wherein linker L is absent.
158. The compound of formula (I) according to any one of clauses 136 to 157, wherein n is 0. 159. The compound of formula (I) according to any one of clauses 136 to 157, wherein n is 1. 160. The compound of formula (I) according to any one of clauses 136 to 159, wherein R¹ and R² are independently selected from H and C₁₋₅ alkyl.
161. The compound of formula (I) according to clause 160, wherein R¹ and R² are independently selected from the group consisting of CH₂CH₃ and CH₃; and in particular are both CH₃.
162. The compound of formula (I) according to any one of clauses 136 to 159, wherein R¹ and R² together with the C atom to which they are attached combine to form a C₄₋₇ cycloalkyl ring or a 4-7 membered heterocyclic ring, both of which are optionally substituted on an available atom by one or more groups selected from C₁₋₂ alkyl and oxo.
163. The compound of formula (I) according to clause 162, wherein R¹ and R² together with the C atom to which they are attached combine to form a cycloheptyl or cyclohexyl ring which is optionally substituted on an available atom by one or more groups selected from C₁₋₂ alkyl and oxo.
164. The chemical entity, surface having a coating, or method according to any one of clauses 136 to 144 or 147 to 163, wherein m is 1-5,000,000, 2-5,000,000, 1-1,000,000, 2-1,000,000, 1-500,000, 2-500,000, 1-400,000, 2-400,000, 1-200,000, 2-200,000, 1-100,000, 2-100,000, 1-50,000, 2-50,000, 1-20,000, 2-20,000, 1-10,000, 2-10,000, 1-5,000, 2-5,000, 1-2,000, 2-2,000, 1-1,000, 2-1,000, 1-500, 2-500, 1-100, 2-100, 1-50, 2-50, 1-20, 2-20, 1-10, 2-10, 1-5 or 2-5.
165. The chemical entity, surface having a coating, or method according to clause 164, wherein m is 2-500,000, 50-500,000, 100-500,000, 500-500,000, 1,000-500,000, 5,000-500,000, 1-200,000, 2-200,000, 50-200,000, 100-200,000, 500-200,000, 1,000-200,000, 5,000-200,000, 1-100,000, 2-100,000, 50-100,000, 100-100,000, 500-100,000, 1,000-100,000, 5,000-100,000, 1-50,000, 2-50,000, 50-50,000, 100-50,000, 500-50,000, 1,000-50,000, 5,000-50,000, 1-20,000, 2-20,000, 50-20,000, 100-20,000, 500-20,000, 1,000-20,000, 5,000-20,000, 1-5,000, 2-5,000, 50-5,000, 100-5,000, 500-5,000, 1,000-5,000, 1-20,000, 2-20,000, 50-20,000, 100-20,000, 500-20,000, 1,000-20,000, 5,000-20,000, 1-1,000, 2-1,000, 50-1,000, 100-1,000, or 500-1,000.
166. The chemical entity, surface having a coating, or method according to clause 165, wherein m is 1-1,000, 2-1,000, 50-1,000, 100-1,000, or 500-1,000.
167. The compound of formula (I) according to clause 166, wherein m is 2, 3 or 4; and in particular is 2 or 3.
168. The compound of formula (I) according to clause 164 or clause 165, wherein m is 5,000-100,000 for example m is 5,000-50,000 or 5,000-20,000.
169. The compound of formula (I) according to clause 136, which is of Formula (Ia):

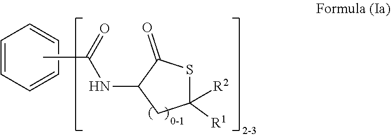

wherein,
R¹ and R² are independently selected from H and C₁₋₅ alkyl, e.g. selected from CH₃ and CH₂CH₃ (and in particular are both CH₃); or R¹ and R² together with the C atom to which they are attached combine to form a cycloheptyl or cyclohexyl ring which is optionally substituted on an available atom by one or more groups selected from C₁₋₂ alkyl and oxo.
170. The compound of Formula (I) according to clause 136, which is of Formula (Iaa):

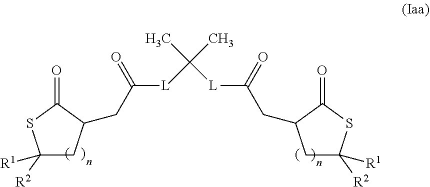

where L, n, R¹ and R² are as defined in clause 1.
171. The compound of Formula (I) according to clause 170, which is of Formula (Iab):

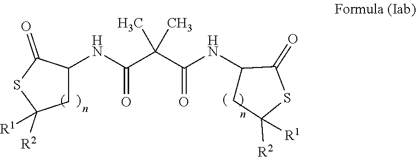

wherein n, R¹ and R² are as defined in clause 1.
172. The compound of Formula (I) according to clause 171, which is of Formula (Iac):

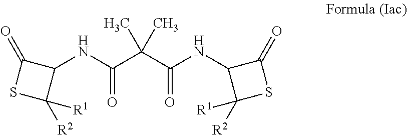

wherein R¹ and R² are as defined in clause 1.
173. A process for the preparation of a compound of Formula (I):

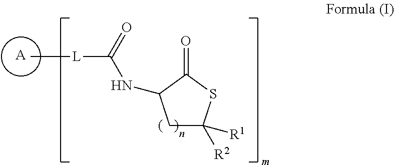

wherein,
A is a core moiety;
L is an optional linker;
R¹ and R² are independently selected from H and C₁₋₅ alkyl; or R¹ and R² together with the C atom to which they are attached combine to form a C₄₋₇ cycloalkyl ring or a 4-7 membered heterocyclic ring, both of which are optionally substituted on an available atom by one or more groups selected from C₁₋₂ alkyl and oxo;
n is 0 or 1;
m is an integer of at least 1;
the process comprising reacting a compound of Formula (IV) with a compound of formula (V) under suitable conditions to form a compound of Formula (I):

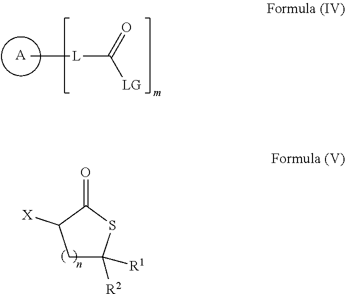

wherein,
for Formula (IV):
LG is a leaving group or an electrophilic group, or moiety C═O-LG is an electrophilic group;
A is a core moiety;
L is an optional linker; and
m is an integer of at least 1;
and for Formula (V):
n is 0 or 1;
R¹ and R² are independently selected from H and C₁₋₆ alkyl; or R¹ and R² together with the C atom to which they are attached combine to form a C₄₋₇ cycloalkyl ring or a 4-7 membered heterocyclic ring, both of which are optionally substituted on an available atom by one or more groups selected from C₁₋₂ alkyl and oxo; and
X is NH₂ or NH₃⁺Y⁻, wherein Y⁻ is a suitable anion e.g. Cl⁻;
174. The process according to clause 173, wherein LG is a leaving group (e.g. selected from the group consisting of halo (such as chloro or bromo), OMs and OTs) or an electrophilic group (e.g. selected from the group consisting of azide, epoxide and isocycanate); or the moiety C═O-LG is an electrophilic group (e.g. moiety C═O-LG comprises an ester (such as a succinimidyl ester), a carbonate (such as a succinimidyl carbonate) or an anhydride).
175. The process according to clause 173 or clause 174, wherein the reaction is carried out in the presence of aqueous base and/or buffer.

Chemical entities and coatings comprising such chemical entities, as described herein, in at least some embodiments, are expected to have one or more of the following merits or advantages:
suitable nitric oxide release characteristics, e.g. as measured using Evaluation Methods Ai and Aii;
good adherence to a surface of a substrate such as the surface of a medical device e.g. as measured using Evaluation Method C;
uniform and uninterrupted coating (i.e. free of flaws) when coated on the surface of a substrate such as the surface of a medical device;
low brittleness when coated on the surface of a substrate such as the surface of a medical device;
compatibility with additional therapeutic agents, such as heparin;
soluble in solvents which are benign and/or suitable in bulk manufacturing settings; and
compatibility with a range of substrate materials conventionally used in the manufacture of medical devices.

### Definitions and Abbreviations

Boc tert-butyloxycarbonyl
DCM dichloromethane
DIPEA diisopropylethylamine
Da Dalton (=g/mol)
EDTA ethylenediaminetetraacetic acid
ePE expanded polyethylene
ePTFE expanded polytetrafluoroethylene
ESI electrospray ionization
GPC gel permeation chromatography
h hour
HFP hexafluoropropylene
HMDA hexamethylenediamine
HPLC high performance liquid chromatography
IPA isopropyl alcohol
LS light scattering
Mₙ number average molecular weight
M_{w} weight average molecular weight
M_{z} z average molecular weight
MALDI matrix assisted laser desorption ionization
Ms methanesulfonyl
MS mass spectrometry
NMP N-methyl-2-pyrrolidone
PBS phosphate buffered saline
PDMS polydimethylsiloxane
PEI polyethyleneimine
PPD p-phenylenediamine
PPI poly(propyleneimine)
Q-ToF quadropole time-of-flight
RT room temperature
ToF-SIMS time-of-flight secondary ion mass spectrometry
Ts toluenesulfonyl
VDF vinylidene fluoride

### EXAMPLES

### General Procedures

### Chemicals

D/L-homocysteine thiolactone HCl salt (Compound (5)) was obtained from Sigma-Aldrich (PN 53530). Benzene-1,3-dicarbonyloxysuccinimide was obtained from Sigma-Aldrich (PN BOG00213). PEI was obtained from Sigma Aldrich, catalogue number 408727 (average Mw˜25,000 by LS, average Mn˜10,000 by GPC, branched). D-penicillamine (Compound (1)) was obtained from Sigma-Aldrich (PN P4875). Isophthaloyl chloride was obtained from Sigma-Aldrich (PN I19403). Benzene-1,3-dicarbonyloxysuccinimide was obtained from Sigma-Aldrich (PN BOG00213). 1,3,5-Benzenetricarbonyl trichloride was obtained from Sigma-Aldrich (PN 147532). VDF-HFP fluoropolymer was obtained from Sigma-Aldrich (PN 427150).

### Materials

Expanded polytetrafluoroethylene (ePTFE) film (C401939418/10932368) was obtained from W. L. Gore & Associates, Inc. Stainless steel foil was obtained from Trinity Brand Industries, Inc. (PN 24S-3165). Pebax® nylon film was obtained from W. L. Gore & Associates (02219-03 TMVR). Expanded polyethylene (ePE) film was made as described in U.S. Pat. No. 9,926,416 or U.S. Ser. No. 10/577,468. Berkeley Life Nitric Oxide Saliva Test Strips were obtained from Berkeley Life Professional, Chicago Illinois.

### Analytical Methods

### NMR Analysis

Samples for ¹H solution NMR collection were prepared by dissolving approximately 15 mg of sample in 1 mL of deuterated solvent. A Bruker BioSpin Neo 300 MHz system was used to collect ¹H NMR data at 300.13 MHz. A Bruker BioSpin 5 mm BBFO probe was installed in a standard bore 7.05T Bruker BioSpin ultra-shielded superconducting magnet. Temperature during NMR acquisition was 300K (26.9° C.). Software used for data acquisition and data processing was Topspin 1.3 or higher (Bruker). The spectra were referenced to the residual proton peak from the deuterated solvent.

### FTIR Spectroscopy

FTIR spectroscopy was carried out on an Agilent 670 spectrometer (Agilent Technologies, Santa Clara, CA USA) or a Thermo Scientific Nicolet iS50 spectrometer (Thermo Fisher Scientific, Waltham, MA USA) equipped with an attenuated total reflectance (ATR) diamond crystal cell. Each spectrum was recorded with at least 32 scans and a resolution of 4 cm⁻¹.

### Liquid Chromatography-Mass Spectrometry

Waters Xevo TQ-S LC Conditions:
Column: 2.1 mm×50 mm, 1.7 m, 130A, ACQUITY UPLC BEH C8
Column Oven: 30° C.
Injection: 5° L
Solvent A: Water+0.1% Formic Acid
Solvent B: Acetonitrile+0.1% Formic Acid

| Gradient: | Time(min) | % B | Flow (mL/min) |
|---|---|---|---|
| | 0.00 | 50.0 | 0.400 |
| | 10.00 | 50.0 | |
| Run time | 10 minutes | | |

Waters PDA Detector Conditions:
PDA Detector Type: UPLC LG 500 nm
Run Time: 10.0 min
Scan Range: 192-400 nm
Sample Rate: 10 points/sec
Resolution: 2.4 nm
Waters Xevo TQ-S LC-MS/MS Conditions:

| ESI Positive or Negative mode | |
|---|---|
| Source (ES): | Analyzer: |
| Capillary (kV): 3.00 | LM1 Resolution: 2.8 |
| Cone (V): 26.00 | HM 1 Resolution: 14.8 |
| Source Offset (V): 50.0 | MS Mode Collision Energy: 4.00 |
| Source Temperature (° C.): 150 | MS/MS Mode Collision Energy: |
| | 33.00 |
| Desolvation Temperature (C): 600 | LM 2 Resolution: 2.8 |
| Collision Gas Flow (mL/min): 0.17 | HM 2 Resolution: 14.6 |
| Nebuliser Gas Flow (Bar): 7.00. | |

### Molecular Weight Measurement by End Group Analysis

An estimated number average molecular weight was determined by a colorimetric method based on ASTM D7409 by carboxylic acid end group titration in n-methyl pyrrolidone at room temperature. Prior to running the carboxylate end group analysis ninhydrin test was performed to show that amine end groups were not present.

### Molecular Weight by Gel Permeation Chromatography

Polymer samples were diluted in dimethylsulfoxide. The diluted solutions were then measured on a Malvern PanAnalytic OmniSec Gel Permeation Chromatography-Light Scattering Instrument using a Dual Angle Light Scattering Detector using a refractive index detector. The gel permeation chromatography column was run in an Agilent 1260 Infinity II liquid chromatography system.

### Evaluation Methods

Ai. Nitric Oxide (NO) Release

The ability of a chemical entity as described herein, or surface having a coating comprising a chemical entity as described herein, to release nitric oxide (NO) is assessed using commercially available Berkely nitric oxide test strips (“Berkeley Life Nitric Oxide Saliva Test Strips”). Instructions for use for such test strips are provided with the packaging of the Berkely nitric oxide test strips, and additionally are described in WO2013/023217A1 and WO2014/039794A2 (incorporated herein by reference).The test strips provide a semi-quantitative measure of NO-derived analytes (primarily aqueous nitrite anion) rather than NO itself, because as is known to the art (Ignarro et al., 1993), aqueous NO has a very short half-life of only seconds, whereas aqueous nitrite anion (which derives from aqueous NO) has a half-life of several hours. As such, the nitrite anion is an appropriate surrogate to measure aqueous NO concentrations. The test strips comprise a nitrite detection pad containing dry chemical reagents responsive to different nitric oxide analytes, thereby, indicating concentrations of nitric oxide. The test strips are capable of detecting a concentration range of the nitric oxide analyte from 0 to 400 μmol/L nitrite with visibly distinct intensities of colorimetric sub-ranges expressed as a red colour (i.e., approximately 700 nm wavelength) of increasing intensity, qualitatively corresponding to micromolarity, specifically, but not limited to: 0 μM, 1 μM to 25 μM, 25 μM to 100 μM, 100 μM to 200 μM, 200 μM to 350 μM, and greater than 400 μM nitrite.

The surface having a coating to be assessed is submerged in a test solution of PBS, 10.0 mM, pH 7.4, with 100 μM EDTA. NO concentration is assessed at given timepoints by dipping the test strip in the solution. After 5 seconds, the test strip is removed, the strip is folded over and the sample pad is gently pressed against the test pad and held for 10 seconds. The ends are separated and compared to the Berkeley Test Nitric Oxide Scale, where the visibly distinct colorimetric sub-ranges comprise 6 subjective levels of intensity of red wavelengths (i.e., approximately 700 nm wavelength), corresponding to 0 μM, 25 μM, 100 μM, 200 μM, 350 μM, and 400 μM of nitric oxide-derived analytes, depending upon the qualitative intensity of red subjectively observed by a user. Thus, once the strip has been allowed to develop, the colour intensity of the strip is subjectively determined and the concentration of nitrate anion (and hence NO release) is determined. As shown in the Examples below, because each strip is assigned to one of the 6 subjective intensity levels (the sixth being no response), the resulting NO release plot has discrete jumps rather than a continuous curve, although in practice continuous release is expected to be occurring.

### Aii Measurement of NO by Thermal Gravimetric Analysis (TGA)

The amount of nitric oxide bound to the thiol functionality is determined by the mass loss by thermal desorption measured on a TGA. Prior to the experiment the identity of the initial thermal desorption (with its onset between 750C and 1020C depending on the monomer unit) is measured by thermal desorption FT-IR. By thermal desorption FT-IR the initial mass loss is characteristic by predominately nitric oxide with a fraction of thiol decomposition product not apparent in the parent non-nitrosylated polymer. The nitric oxide amount is determined by TGA using a Q500 TGA by TA instruments. The sample is heated from room temperature to 400° C. using a high resolution scan at 5° C./minute until a mass loss is detected. The NO mass loss is calculated from the derivative curve with the first mass loss to the inflection point between the first and second mass loss peaks in the derivative of mass loss curves.

### Aiii NO Elution Measurements

Absorbance NO Measurement with Griess Reagent

Samples previously weighed are added to scintillation vials. To the vials is added 15 mL of 10 mM PBS with 100 μM EDTA (pH 7.2). The samples are then incubated at 37° C. protected from light for specified timepoints. At the time points 50 μL of elution media is removed and added to a 96 well plate. Six standard solutions of sodium nitrite in 10 mM PBS with 100 μM EDTA (pH 7.2) (140, 70, 30, 10, 2 and 0 μM sodium nitrite) are then added to the 96 well plate. To each sample and standard is added 50 μL of PBS and 50 μL each of Griess Reagent A and B (VWR product number 102965-266). The 96 well plate is then incubated at room temperature (22-25° C.) for 10 minutes, and then the absorbance spectra at 540 nm is taken on a Molecular Devices SpectraMax 190 plate reader. A calibration curve is calculated from the sodium nitrite standards, and the elution time points are calculated using the calibration standard curve fitting parameter.

### NO Measurement by NO Analyzer

Nitric oxide elution is measured using a Thermo Scientific 42i NOₓ Analyzer. The nitrosylated polymer samples are added to PBS and the solution is heated to 37° C. Measurements are taken on Days 1, 2, 3, 7 and then once weekly thereafter.

A standard solution of 5 mM sodium nitrite is prepared. Five different volumes (10-50 uL) of each standard are injected into the sample chamber containing a solution of vanadium trichloride and HCl, which converts the nitrite to nitric oxide and is passed into the NOx Analyzer under flow of argon. The chemiluminescence signal from the reaction of nitric oxide with ozone is then detected.

The samples are added to fresh PBS at 37° C. within the sample chamber. Headspace analysis is performed wherein the PBS solution containing a sample was sealed in the vessel for a set amount of time and then the system is opened and a flow of argon is introduced to the system to drive the released Nitric Oxide through the NOx Analyzer. The nitric oxide released is analyzed using the chemiluminescence signal and the amount is determined using the calibration curve prepared.

### B. Stability to Ethylene Oxide (EO)

A sample of a chemical entity as described herein or of a surface having a coating comprising a chemical entity as described herein is placed in a breathable polyethylene pouch (e.g. a Tyvek pouch) and subjected to at least 12 hours preconditioning at 43° C. and 65% relative humidity followed by 12 hours exposure to 600 mg/L ethylene oxide at 52° F. and 25% relative humidity. The chamber is then aerated at 32° F. for at least 12 hours until ethylene oxide concentration was less than 0.25 ppm. After sterilization, the NO release profile of the sample or surface is assessed using Evaluation Method Ai.

### C. Adherence to Medical Device

Adhesion testing of the NO-releasing coating onto a surface, involves nanoscratch testing, imaging of the scratch region, and nanoindent testing, to record quasi-static reduced modulus values, which are then converted into adhesive critical failure load values (a routine test procedure well known to the art). Testing is carried out using a Hysitron 950 Triboindenter (Bruker Inc., Eden Prairie, Minn.). Five samples of each nitric oxide-releasing coating formulation are tested at random locations. This technique is used to investigate the amount or thickness or distribution of the coating over the surface, and determine where the amount or thickness or distribution of the coating is variable over the surface. This technique is also used to examine a coating comprising one or more additional coatings, by measuring the modulus and/or adhesion strength of the coating as a function of the nanoindentation depth. Furthermore, this technique is also used to examine the incorporation of a therapeutic agent in the coating. Along with nanomechanical testing, ASTM D3359 “Test Methods for Measuring Adhesion by Tape” can be used to look at adhesion of the NO releasing coating from the underlying substrate.

### General Procedures

A. Synthesis of Compounds of Formula (I)—from an Acid Chloride

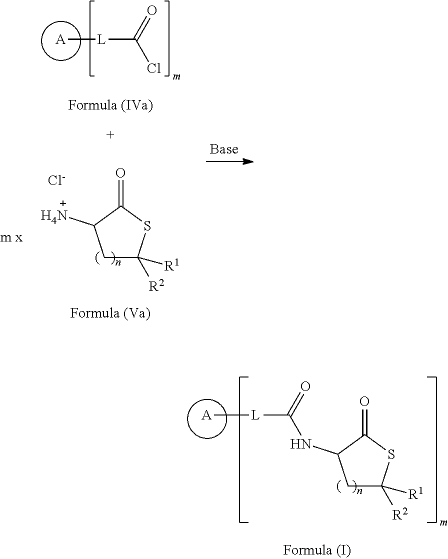

To a stirred CHCl₃ or DCM solution/suspension of poly-functional acid chloride (compound of Formula (IVa), e.g. obtained from sources described in the following Examples) and thiolactone-HCl salt (compound of formula (Va), e.g. compound (4) prepared as described in Example 1, or compound (5) which is commercially available), in the appropriate stoichiometric ratio (m×), was added dropwise via syringe, 2 eq. of DIPEA per eq. of compound of formula (Va). After several minutes, the solution became clear, and was stirred for 30 more minutes. The solution was then washed with 1 M HCl (3×), followed by water (1×). The organic layer was then dried over anhydrous Na₂SO₄, filtered, and the solvent rotary evaporated under reduced pressure to give the compound of formula (I), typically as a white, microcrystalline powder.

B. Synthesis of Compounds of Formula (I)—from a Succinimidyl Ester

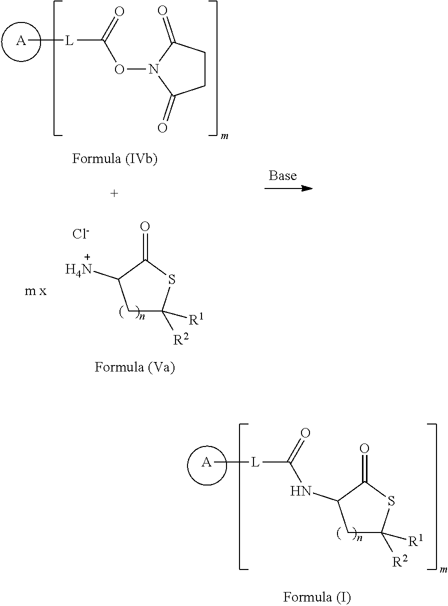

To a vigorously stirred MeCN solution/suspension of poly-functional succinimidyl ester (compound of Formula (IVb), obtained from sources described in the following Examples) and thiolactone-HCl salt (compound of formula (Va), e.g. compound (4) prepared as described in Example 1, or compound (5) which is commercially available) in the appropriate stoichiometric molar ratio (m×), was added an equivalent volume of brine (250 g/L NaCl). To the resultant emulsion, 1 M sodium carbonate was added all at once, and the mixture vigorously stirred for 15 minutes at RT, after which, the layers were allowed to separate. The aqueous layer was discarded, and the organic layer was washed two more times with brine and dried over anhydrous Na₂SO₄. The solution was then filtered, and the solvent rotary evaporated under reduced pressure to give the compound of formula (I), typically as a white, microcrystalline powder.

### Synthesis of Intermediates

For all Examples shown below, where the structures contain one or more chiral centres, the absolute stereochemistry is not shown, but is indicated in the intermediate or chemical entity name, or can be derived from the indicated stereochemistry of the starting material. It should be noted that where an Example relates to a D-stereoisomer, the process described is also suitable for synthesizing the L-stereoisomer, and mixtures (including racemic mixtures) of the D- and L-stereoisomer, and where an Example relates to a mixture of D- and L-stereoisomers, the process described is also suitable for synthesizing the L-stereoisomer and D-stereoisomer individually. When structures contain more than one chiral centre, in certain cases a number of different diastereomers can be formed, depending on the number of chiral centres. Such structures may be chiral or non-chiral (meso).

### Example 1: Synthesis of D-Penicillamine Thiolactone HCl Salt (4)

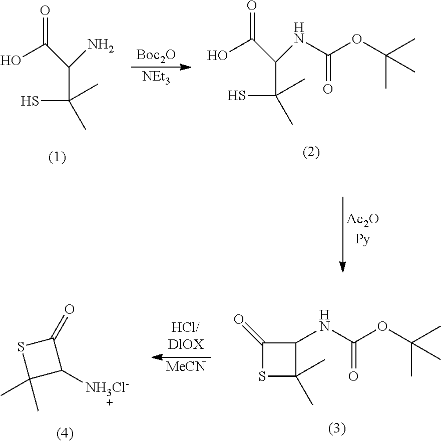

D-penicillamine (1) was N-protected with a Boc group as described in the procedure set out in EP1165532A1 (incorporated herein by reference) for L-penicillamine. Specifically, D-penicillamine ((1), 15 g, 0.1 mol) was suspended in methanol (250 ml) and cooled to 0° C. Triethylamine (14 ml, 0.1 mol) was added, followed by solid di-tert-butyl dicarbonate (24 g, 0.11 mol) all at once. The reaction mixture was stirred at 25° C. overnight, after which the solvents were removed by rotary evaporation to produce a residue. The residue was dissolved in EtOAc (100 ml) and washed with 1 M HCl (100 ml), water (50 ml) and brine (50 ml), dried over anhydrous MgSO₄, filtered, and rotary evaporated under reduced pressure down to a clear, viscous residue. Approximately 20 mL of hexane was added to the viscous residue to form an azeotrope with the residual EtOAc and the solvents rotary evaporated under reduced pressure to give Boc-protected penicillamine (2) as a fluffy, white solid.

Compound (2) was cyclized to the corresponding thiolactone (3) based on a procedure described in Hopkins et al. (2018) for the synthesis of N-acetylpenicillamine. Specifically, compound (2) was dissolved in 40 mL of pyridine and cooled to 0° C. on an ice bath. Meanwhile, 40 mL of pyridine and 40 mL of acetic anhydride were mixed in a separate container and placed in a freezer (−5° C.). Both solutions were allowed to cool for 0.5 h before being combined and allowed to stir overnight at RT. The combined solution was then rotary evaporated under reduced pressure at 40° C. until a crude solid remained. The crude solid was then dissolved in 80 mL of CHCl₃ and washed with 1 M HCl (3×50 ml), and the organic and aqueous layers allowed to separate. The organic layer was isolated and dried over anhydrous MgSO₄ and filtered. The CHCl₃ was removed under rotary evaporation to give compound (3) as a white solid and dried overnight under vacuum.

The deprotection step to yield ammonium salt (4) was carried out by dissolving compound (3) in 200 mL of MeCN at 25° C. 37.5 mL of 4.0 M HCl in dioxane (1.5 eq.) was then added to the solution dropwise via syringe with stirring. The solution initially became clear, then after approximately 5-10 minutes, a reaction product began precipitating to give a white, cloudy suspension. The suspension was stirred for 45 minutes, 100 mL of Et₂O was added, and the suspension stirred 5-10 minutes more. A white, microcrystalline product was collected on a frit and washed with additional Et₂O and dried under vacuum overnight to yield D-penicillamine thiolactone HCl salt (4).

Synthesis of compounds of Formula (I)

### Example 2: Synthesis of N¹,N⁶-bis(2,2-dimethyl-4-oxothietan-3-yl)adipamide (6)

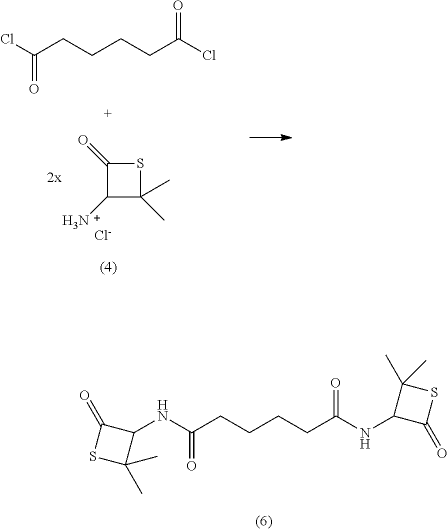

Adipoyl chloride (2.4 g, 0.013 mol) was reacted with two equivalents of D-penicillamine thiolactone HCl salt (Example 1, compound (4), 4.4 g, 0.026 mol) according to General Procedure A. Title compound (6) was isolated as a white solid. ¹H NMR (400 MHz, DMF-d,) d 8.92 (d, 2H, J=8.67 Hz), 5.76 (d, 2H, J=8.79 Hz), 2.29 (m, 4H), 1.79 (s, 6H), 1.67 (s, 6H), 1.62 (m, 4H). FTIR-ATR (cm⁻¹) vₘₐₓ=1754.

### Example 3: Synthesis of N¹,N⁸-bis(2-oxotetrahydrothiophen-3-yl)octanediamide (7)

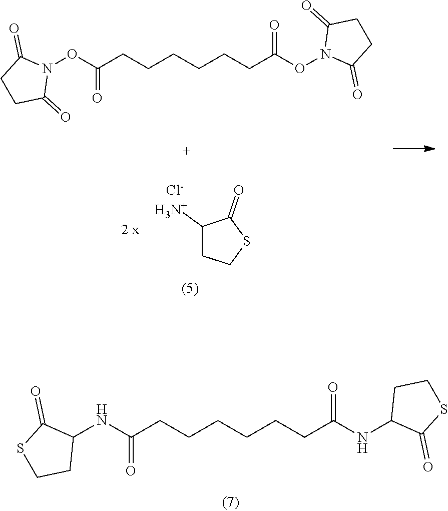

Disuccinimidyl suberate (1.0 g, 2.7 mmol) was reacted with two equivalents of D/L-homocysteine thiolactone HCl salt (Compound (5), 0.83 g, 5.4 mmol) according to General Procedure B. Title compound (7) was isolated as a white solid. ¹H NMR (400 MHz, DMSO-d₆) δ 8.12 (d, 2H, J=8.41 Hz), 4.59 (m, 2H), 3.44-3.22 (m, 4H), 2.40 (m, 2H), 2.14-1.97 (m, 6H), 1.48 (m, 4H), 1.25 (m, 4H). FTIR-ATR (cm⁻¹) vₘₐₓ=1648.

D/L-homocysteine thiolactone HCl salt (Compound (5) may be obtained by the method of Martens et al, 1991.

### Example 4: Synthesis of N¹,N³-bis(2,2-dimethyl-4-oxothietan-3-yl)isophthalamide (8)

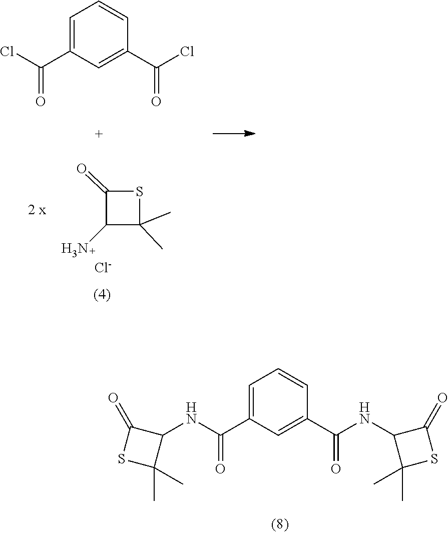

Isophthaloyl chloride (500 mg, 2.46 mmol) was reacted with two equivalents of D-penicillamine thiolactone HCl salt (Example 1, compound (4), 826 mg, 4.92 mmol) according to General Procedure A. Title compound (8) was isolated as a white solid. ¹H NMR (400 MHz, CDCl₃) δ 8.09 (d, 2H, J=8.82 Hz), 7.92 (t, 1H, J=1.47 Hz), 7.84 (dd, 2H, J=7.79, 1.67 Hz), 7.41 (t, 1H, J=7.77 Hz), 5.96 (d, 2H, J=8.86 Hz), 1.93 (s, 6H), 1.79 (s, 6H). FTIR-ATR (cm⁻¹) vₓ=1748.

### Example 5: Synthesis of N¹,N³-bis(2-oxotetrahydrothiophen-3-yl)isophthalamide (9)

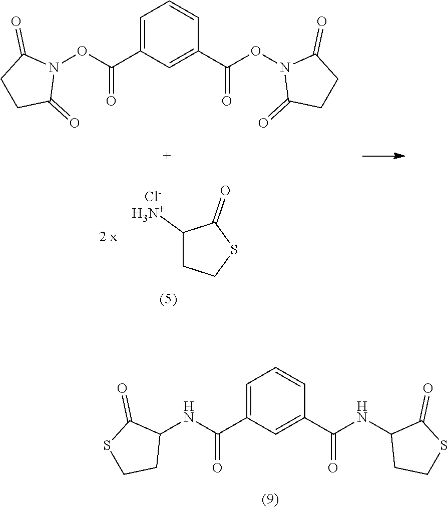

Benzene-1,3-dicarbonyloxysuccinimide (2.0 g, 5.6 mmol) was reacted with two equivalents of D/L-homocysteine thiolactone HCl salt (Compound (5), 1.7 g, 11 mmol) according to the method set out in General Procedure B. Title compound (9) was isolated as a white solid. ¹H NMR (400 MHz, DMSO-d₆) δ 8.79 (d, 2H, J=8.2 Hz), 8.37 (t, 1H, J=1.67 Hz), 8.01 (dd, 2H, J=5.97, 1.80 Hz), 7.59 (t, 1H, J=7.75 Hz), 4.84 (m, 2H), 3.53-3.31 (m, 4H), 2.58-2.47 (m, 1H), 2.35 (m, 3H). FTIR-ATR (cm⁻¹) vₘₐₓ=1694.

### Example 6: Synthesis of N¹,N³,N⁵-tris(2,2-dimethyl-4-oxothietan-3-yl)benzene-1,3,5-tricarboxamide (10)

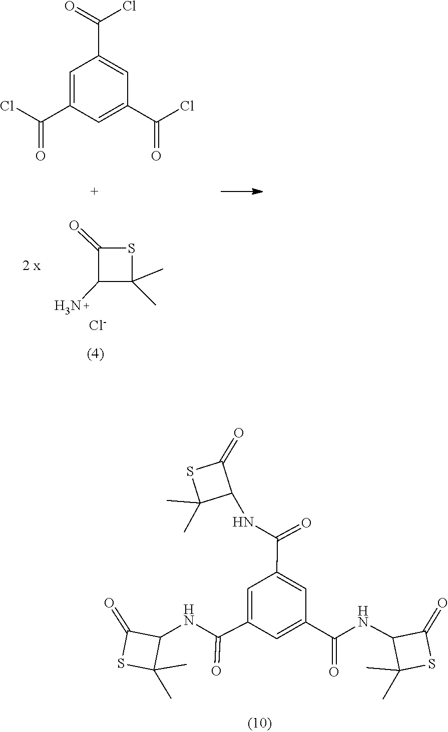

1,3,5-Benzenetricarbonyl trichloride (500 mg, 1.9 mmol) was reacted with three equivalents of D-penicillamine thiolactone HCl salt (Example 1, compound (4), 950 mg, 5.7 mmol) according to General Procedure A, except that in this case, the compound (10) precipitated from the reaction solution. After 2 hours of stirring, the product was collected on a frit and washed with 1M HCl followed by water then dried. Title compound (10) was isolated as a white solid. ¹H NMR (400 MHz, DMF-d₇) d 9.94 (d, 3H, J=8.38 Hz), 8.77 (s, 3H), 6.02 (d, 3H, J=8.46 Hz), 1.92 (s, 9H), 1.79 (s, 9H). FTIR-ATR (cm⁻¹) vₘₐₓ=1655.

### Example 7: Synthesis of N¹,N³,N⁵-tris(2-oxotetrahydrothiophen-3-yl)benzene-1,3,5-tricarboxamide (11)

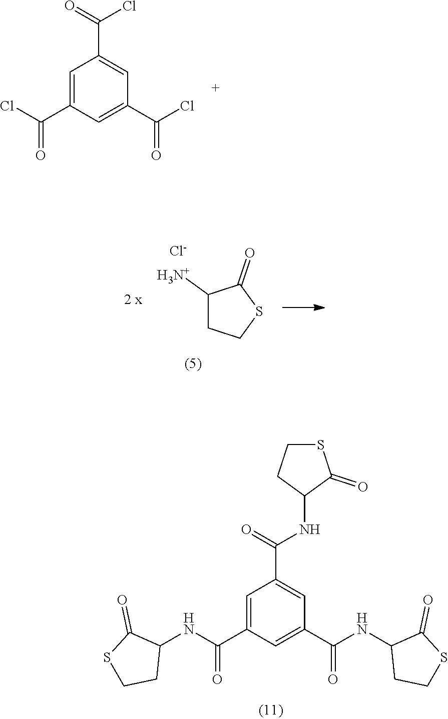

1,3,5-Benzenetricarbonyl trichloride (3.4 g, 12.8 mmol) was reacted with three equivalents of D/L-homocysteine thiolactone HCl salt (Compound (5), 5.9 g, 38 mmol) according to General Procedure B. Title compound (11) was isolated as a white solid. ¹H NMR (400 MHz, DMSO-d₆) δ 9.16 (d, 3H, J=8.32 Hz), 8.50 (s, 3H), 4.90 (m, 3H), 3.55-3.42 (m, 3H), 3.40-3.28 (m, 3H), 2.57-2.46 (m, 3H), 2.42-2.25 (m, 3H). FTIR-ATR (cm⁻¹) vₘₐₓ=1638.

### Example A1: Synthesis of N¹,N³-bis(2,2-dimethyl-4-oxothietan-3-yl)-2,2-dimethylmalonamide (31)

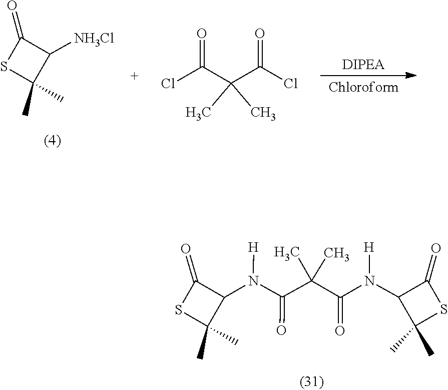

To a 500 mL roundbottom flask was added 10 g of 2,2′-dimethylmalonyl chloride, 20.8 g of D-penicillamine thiolactone hydrochloride (Example 1, compound (4), with 200 mL of chloroform. To an addition funnel was added 42 mL of di-isopropylethylamine (DIPEA). The reactor was purged with argon and cooled to 4° C. with an ice bath. The DIPEA was added dropwise to the thiolactone suspension. With the addition of the DIPEA the reaction solution was then allowed to slowly come to room temperature and stirred for 1 hour. The reaction media was then added to a separatory funnel and extracted with 1.2 L of 1N hydrochloric acid. The chloroform solution was dried over magnesium sulfate and filtered. The chloroform was evaporated on a rotary evaporator until 50 mL of chloroform remained. To the solution was added 25 mL of diethyl ether resulting in the crystallization of the product. The title compound (31) was collected on a glass frit and then dried under vacuum. ¹H NMR δ1.61 (s) δ1.70 (s) δ1.95 (s) δ5.65 (d) δ7.54 (d).

### Hydrolysis and S-Nitrosothiol Derivatization of Compound of Formula (I)

### Test Example 8: Hydrolysis and S-nitrosothiol Derivatization of Compound 6 to form 2,2′-(adipoylbis(azanediyl))bis(3-methyl-3-(nitrosothio)butanoic acid) (13)

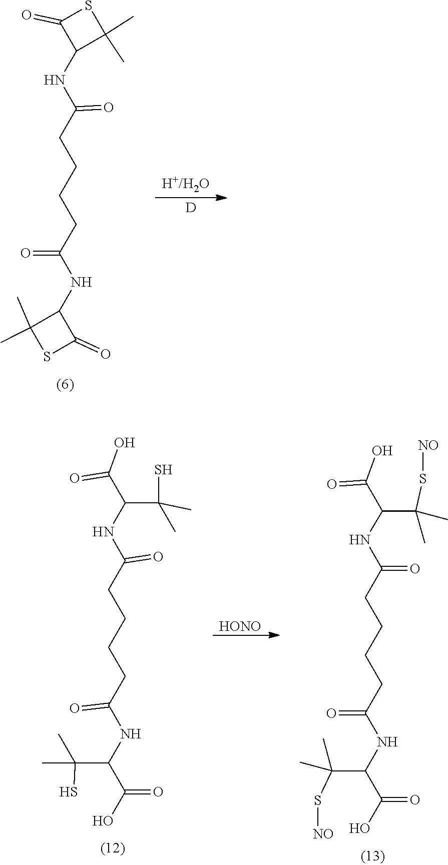

200 mg of N¹,N⁶-bis(2,2-dimethyl-4-oxothietan-3-yl)adipamide (Example 2, compound (6)) was suspended in 5 mL of water and a few drops of concentrated HCl added. The suspension was heated at reflux for 2 h to give a clear solution of the hydrolysis product compound (12) (2,2′-(adipoylbis(azanediyl))bis(3-mercapto-3-methylbutanoic acid)) ESI-MS m/z calculated for C₁₆H₂₈N²O₆S₂ 408.1 (M−H)⁻; found, 407.0.

After cooling to RT, water and HCl were added to the clear solution to a final volume of 10 mL at 1 M in HCl, then cooled to 0° C. 1 mL of conc. H₂SO₄ was added to form a suspension, and the suspension was cooled to 0° C. in an ice bath. Sodium nitrite (3 eq. per thiol residue of compound (6)) was dissolved in a few milliliters of water (forming nitrous acid (HONO) in situ) and added all at once to the suspension. The ice bath was removed, and the suspension stirred in the dark for 1 h. To the resulting green suspension was added 30 mL of DCM, and the green suspension was vigorously stirred as an emulsion for 15-20 minutes. The emulsion was then poured into a separatory funnel and the bright green, DCM layer was poured over Na₂SO₄ and placed in a refrigerator (5° C.) for 30 minutes. After filtering the DCM layer, the DCM solvent was rotary evaporated under reduced pressure to give the S-nitrosothiol derivative compound (13) as a green solid. Compound (13): ¹H NMR (400 MHz, DMSO-d₆) δ 13.13 (s, 2H), 8.40 (d, 2H, J=9.51 Hz), 5.18 (d, 2H, J=9.51 Hz), 2.15 (m, 4H), 1.96 (s, 6H), 1.93 (s, 6H), 1.40 (m, 4H).

### Reaction of Components B and C to Form “Thiol/—SH” Chemical Entities

### Example 9: Reaction of compound 6 with benzylamine to form N1,N6-bis(1-(benzylamino)-3-mercapto-3-methyl-1-oxobutan-2-yl)adipamide (14)

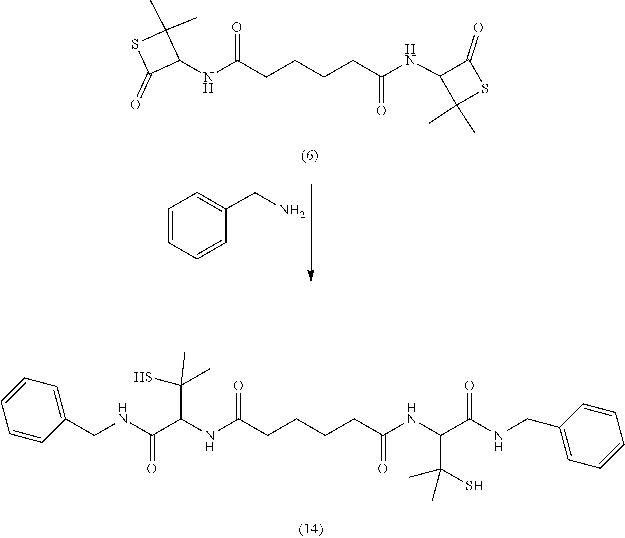 [SMILES: CC1(C)SC(=O)C1NC(=O)CCCCC(=O)NC1C(=O)SC1(C)C ; NCc1ccccc1 ; CC(C)(S)C(NC(=O)CCCCC(=O)NC(C(=O)NCc1ccccc1)C(C)(C)S)C(=O)NCc1ccccc1]

To a stirred suspension of N1,N6-bis(2,2-dimethyl-4-oxothietan-3-yl)adipamide (Example 2, compound (6)) in CHCl₃, was added a 10% excess of benzylamine (based on molar % of compound (6)). The suspension was stirred at RT overnight, and the resulting clear solution was washed twice with 1 M HCl. The organic layer was dried over Na₂SO₄, filtered, the solvent rotary evaporated under reduced pressure to give compound (14) as a white solid. ¹H NMR (400 MHz, CDCl₃) δ 7.86 (t, 2H, J=5.55 Hz), 7.37 (d, 2H, J=9.61 Hz), 7.19 (m, 6H), 7.11 (m, 4H), 4.88 (d, 2H, J=9.70 Hz), 4.29 (dd, 2H, J=15.02, 8.85 Hz), 3.99 (dd, 2H, J=15.02. 9.94 Hz), 2.47 (s, 2H), 2.33 (m, 4H), 1.79-1.52 (m, 4H), 1.49 (s, 6H), 1.36 (s, 6H). ESI-MS m/z calculated for C₃₀H₄₂N₄O4S₂ 586.3 (M−H)⁻; found, 586.2. FTIR-ATR (cm⁻¹) vₘₐₓ=1640.

### Example 10: Reaction of compound 6 with aniline to form N1,N6-bis(3-mercapto-3-methyl-1-oxo-1-(phenylamino)butan-2-yl)adipamide (15)

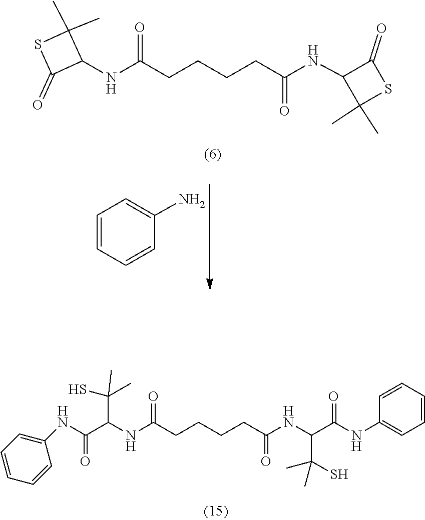 [SMILES: CC1(C)SC(=O)C1NC(=O)CCCCC(=O)NC1C(=O)SC1(C)C ; Nc1ccccc1 ; CC(C)(S)C(NC(=O)CCCCC(=O)NC(C(=O)Nc1ccccc1)C(C)(C)S)C(=O)Nc1ccccc1]

To a stirred suspension of N¹,N⁶-bis(2,2-dimethyl-4-oxothietan-3-yl)adipamide (Example 2, compound (6)) in CHCl₃, was added, a 3-fold excess of aniline (based on molar % of compound (6)). The suspension was stirred at 25° C. in the dark for 3 days, and the resulting clear solution was washed with twice with 1 M HCl. The organic layer was dried over Na₂SO₄, filtered, the solvent rotary evaporated under reduced pressure to give compound (15) as a white solid. ¹H NMR (400 MHz, CDCl₃) δ 9.17 (s, 2H), 7.40-7.30 (m, 6H), 7.13, (m, 4H), 7.01 (t, 2H, J=7.35 Hz), 4.98 (d, 2H, J=9.42 Hz), 2.57 (s, 2H), 2.31 (m, 4H), 1.78-1.50 (m, 4H), 1.60 (s, 6H), 1.43 (s, 6H).

ESI-MS m/z calculated for C₂₈H₃₈N₄O₄S₂ 558.2 (M−H)⁻; found, 557.6. FTIR-ATR (cm⁻¹) vₘₐₓ=1641.

### Example 11: Reaction of compound 6 with morpholine to form N1,N6-bis(3-mercapto-3-methyl-1-morpholino-1-oxobutan-2-yl)adipamide (6a)

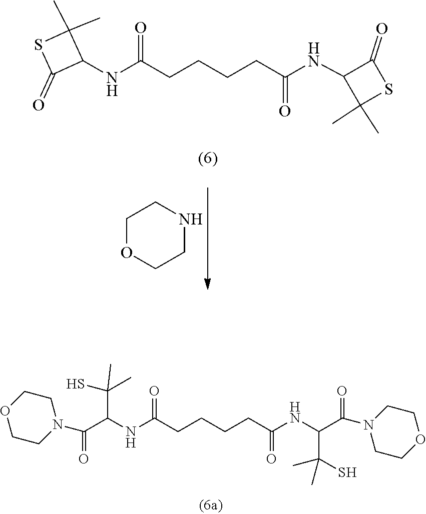 [SMILES: CC1(C)SC(=O)C1NC(=O)CCCCC(=O)NC1C(=O)SC1(C)C ; C1COCCN1 ; CC(C)(S)C(NC(=O)CCCCC(=O)NC(C(=O)N1CCOCC1)C(C)(C)S)C(=O)N1CCOCC1]

To a stirred suspension of adipoyl-1,6-dipenicillamine dithiolactone (Example 2, compound (6)) in chloroform, was added, a 3-fold excess of morpholine (based on molar % of compound (6)). The suspension was stirred overnight at RT, and the resulting clear solution was washed with twice with 1 M HCl. The organic layer was dried over Na₂SO₄, filtered, the solvent rotary evaporated under reduced pressure to give compound (6a) as a white solid. ¹H NMR (400 MHz, DMSO-d₆) δ 8.08 (d, 2H, J=9.42 Hz), 4.96 (d, 2H, J=9.43 Hz), 3.68-3.40 (m, 16H), 2.94 (s, 2H), 2.19 (m, 4H), 1.46 (m, 4H), 1.36 (s, 6H), 1.32 (s, 6H). ESI-MS m/z calculated for C₂₄H₄₂N₄O₆S₂ 546.3 (M−H)⁻; found, 545.1. FTIR-ATR (cm⁻¹) vₘₐₓ=1618.

### Example 12: Reaction of compound 8 with benzylamine to form N1,N3-bis(1-(benzylamino)-3-mercapto-3-methyl-1-oxobutan-2-yl)isophthalamide (8a)

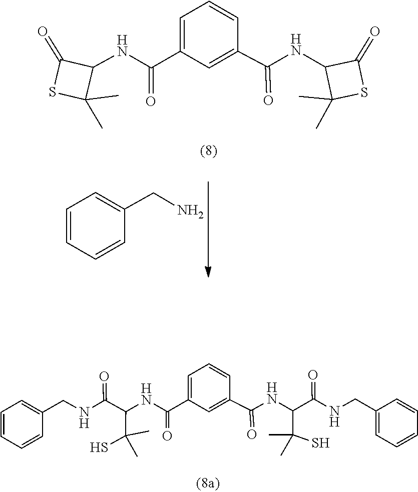 [SMILES: CC1(C)SC(=O)C1NC(=O)c1cccc(C(=O)NC2C(=O)SC2(C)C)c1 ; NCc1ccccc1 ; CC(C)(S)C(NC(=O)c1cccc(C(=O)NC(C(=O)NCc2ccccc2)C(C)(C)S)c1)C(=O)NCc1ccccc1]

To a stirred suspension of N¹,N³-bis(2,2-dimethyl-4-oxothietan-3-yl)isophthalamide (Example 4, compound (8)) in CHCl₃, was added a 10% excess of benzylamine (based on molar % of compound (8)). The solution/suspension was stirred at RT overnight, and the resulting clear solution was washed twice with 1M HCl. The organic layer was dried over Na₂SO₄, filtered, and the solvent rotary evaporated under reduced pressure to give compound (8a) as a white solid. ¹H NMR (400 MHz, DMSO-d₆) δ 8.74, (t, 2H, J=5.83 Hz), 8.33-8.25 (m, 3H), 8.03 (dd, 2H, J=7.76, 1.63 Hz), 7.59 (t, 1H, J=7.76 Hz), 7.37-7.17 (m, 10H), 4.79 (d, 2H, J=9.35 Hz), 4.33 (d, 4H, J=5.80 Hz), 2.97 (s, 2H), 1.46 (s, 6H), 1.43 (s, 6H). ESI-MS m/z calculated for C₃₂H₃₈N₄O₄S₂ 606.2 (M−H)⁻; found, 604.9. FTIR-ATR (cm⁻¹) vₘₐₓ=1636.

### Example 13: Reaction of compound 8 with aniline to form N1,N3-bis(3-mercapto-3-methyl-1-oxo-1-(phenylamino)butan-2-yl)isophthalamide (8b)

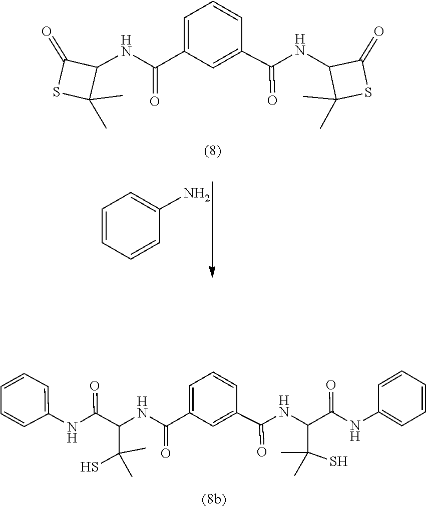 [SMILES: CC1(C)SC(=O)C1NC(=O)c1cccc(C(=O)NC2C(=O)SC2(C)C)c1 ; Nc1ccccc1 ; CC(C)(S)C(NC(=O)c1cccc(C(=O)NC(C(=O)Nc2ccccc2)C(C)(C)S)c1)C(=O)Nc1ccccc1]

To a stirred suspension of N¹,N³-bis(2,2-dimethyl-4-oxothietan-3-yl)isophthalamide (Example 4, compound (8)) in chloroform, was added, a 3-fold excess of aniline (based on molar % of compound (8)). The suspension was stirred overnight at RT, and the resulting clear solution was washed with twice with 1 M HCl. The organic layer was dried over Na₂SO₄, filtered, the solvent rotary evaporated under reduced pressure to give compound (8b) as a white solid. ¹H NMR (400 MHz, DMSO-d₆) δ 10.31 (s, 2H), 8.45, (d, 2H, J=9.12 Hz), 8.29 (t, 1H, J=1.54 Hz), 8.04 (dd, 2H, J=7.75, 1.72 Hz), 7.66-7.56 (m, 5H), 7.32 (m, 4H), 7.07 (t, 2H, J=7.38 Hz), 4.96 (d, 2H, J=9.13 Hz), 3.00 (s, 2H), 1.53 (s, 6H), 1.49 (s, 6H). ESI-MS m/z calculated for C₃₀H₃₄N₄O₄S₂ 578.2 (M−H)⁻; found, 577.0. FTIR-ATR (cm⁻¹) vₘₐₓ=1497.

### Example 14: Reaction of compound 8 with morpholine to form N1,N3-bis(3-mercapto-3-methyl-1-morpholino-1-oxobutan-2-yl)isophthalamide (8c)

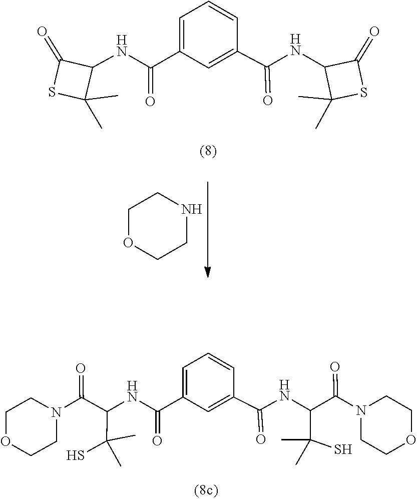 [SMILES: CC1(C)SC(=O)C1NC(=O)c1cccc(C(=O)NC2C(=O)SC2(C)C)c1 ; C1COCCN1 ; CC(C)(S)C(NC(=O)c1cccc(C(=O)NC(C(=O)N2CCOCC2)C(C)(C)S)c1)C(=O)N1CCOCC1]

To a stirred suspension of N¹,N³-bis(2,2-dimethyl-4-oxothietan-3-yl)isophthalamide (Example 4, compound (8)) in chloroform, was added a 3-fold excess of morpholine (based on molar % of compound (8)). The suspension was stirred overnight at RT, and the resulting clear solution was washed with twice with 1 M HCl. The organic layer was dried over Na₂SO₄, filtered, the solvent rotary evaporated under reduced pressure to give compound (8c) as a white solid. ¹H NMR (400 MHz, DMSO-d₆) δ 8.59 (d, 2H, J=9.04 Hz), 8.25 (t, 1H, J=1.51 Hz), 8.02 (dd, 2H, J=7.76, 1.72 Hz), 7.56 (t, 1H, J=7.76 Hz), 5.18 (d, 2H, J=9.03 Hz), 3.79-3.44 (m, 16H), 3.13 (s, 2H), 1.47 (s, 6H), 1.44 (s, 6H). ESI-MS m/z calculated for C₂₆H₃₈N₄O₆S₂ 566.2 (M−H)⁻; found, 566.2. FTIR-ATR (cm⁻¹) vₘₐₓ=1625.

### Example 15: Reaction of compound 10 with benzylamine to form N1,N3,N5-tris(1-(benzylamino)-3-mercapto-3-methyl-1-oxobutan-2-yl)benzene-1,3,5-tricarboxamide (10a)

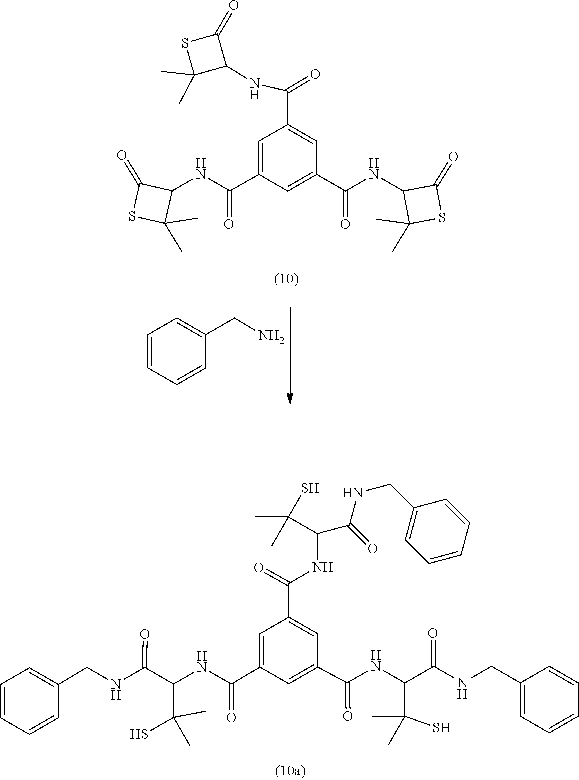 [SMILES: CC1(C)SC(=O)C1NC(=O)c1cc(C(=O)NC2C(=O)SC2(C)C)cc(C(=O)NC2C(=O)SC2(C)C)c1 ; NCc1ccccc1 ; CC(C)(S)C(NC(=O)c1cc(C(=O)NC(C(=O)NCc2ccccc2)C(C)(C)S)cc(C(=O)NC(C(=O)NCc2ccccc2)C(C)(C)S)c1)C(=O)NCc1ccccc1]

To a stirred suspension of N¹,N³, N⁵-tris(2,2-di methyl-4-oxothietan-3-yl) benzene-1, 3,5-tricarboxamide (Example 6, Compound (10)) in chloroform, was added a 10% excess of benzylamine (based on molar % of compound (10)). The suspension was stirred overnight at RT, and the resulting clear solution was washed with twice with 1 M HCl. The organic layer was dried over Na₂SO₄, filtered, the solvent rotary evaporated under reduced pressure to give compound (10a) as a white solid. ¹H NMR (400 MHz, CDCl₃) δ 8.21 (s, 3H), 7.92 (d, 3H, J=8.75 Hz), 7.46, (m, 3H), 7.31-7.19 (m, 15H), 4.80 (d, 3H, J=8.80 Hz), 4.60 (dd, 3H, J=14.70, 6.20 Hz), 4.28 (dd, 3H, J=14.69, 4.56 Hz), 2.50 (s, 3H), 1.6 (s, 9H), 1.41 (s, 9H). ESI-MS m/z calculated for C₄₅H₅₄N₆O₆S₃ 870.3 (M−H)⁻; found, 869.2. FTIR-ATR (cm⁻¹) vₘₐₓ=1643.

### Example 16: Reaction of Compound 10 with Morpholine to Form N1,N3,N5-Tris(3-Mercapto-3-methyl-1-morpholino-1-oxobutan-2-yl)benzene-1,3,5-tricarboxamide (10b)

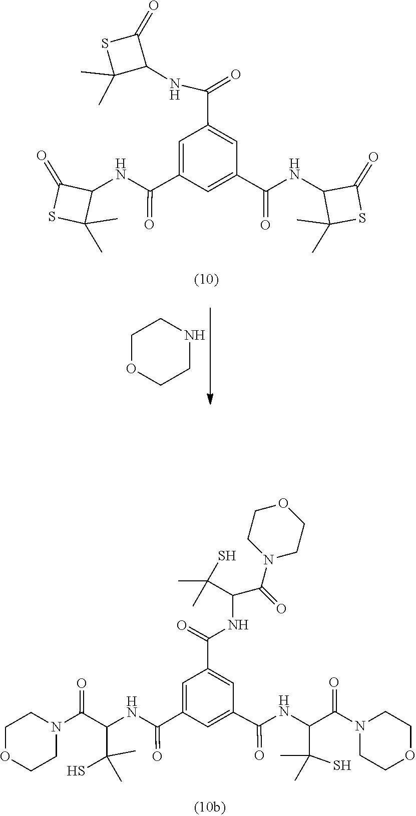 [SMILES: CC1(C)SC(=O)C1NC(=O)c1cc(C(=O)NC2C(=O)SC2(C)C)cc(C(=O)NC2C(=O)SC2(C)C)c1 ; C1COCCN1 ; CC(C)(S)C(NC(=O)c1cc(C(=O)NC(C(=O)N2CCOCC2)C(C)(C)S)cc(C(=O)NC(C(=O)N2CCOCC2)C(C)(C)S)c1)C(=O)N1CCOCC1]

To a stirred suspension of N1,N3,N5-tris(2,2-dimethyl-4-oxothietan-3-yl)benzene-1,3,5-tricarboxamide (Example 6, Compound (10)) in chloroform, was added a 3-fold excess of morpholine (based on molar % of compound (8)). The suspension was stirred overnight at RT to give compound (10b) as a white solid. ¹H NMR (400 MHz, DMSO-d₆) δ 8.81 (d, 3H, J=9.01 Hz), 8.36 (s, 3H), 5.20 (d, 3H, J=9.03 Hz), 3.80-3.44 (m, 24H), 3.13 (s, 3H), 1.48 (s, 9H), 1.45 (s, 9H). ESI-MS m/z calculated for C₃₆H₅₄NO₉S₃ 810.3 (M−H)⁻; found, 809.0. FTIR-ATR (cm⁻¹) vₘₐₓ=1627.

### Polymer Examples

The following examples relate to polymers. The structures provided for the polymers in the schemes below show the polymer chains without the terminal groups, where n represents the number of repeating units. Where a figure is provided for the number of monomer units, this represents an average number of monomer units in the polymer and the units may be present in any order in the polymer.

### Example 17: Copolymerization of Compound 6 with Hexamethylenediamine (HMDA) to Form Copolymer (19)

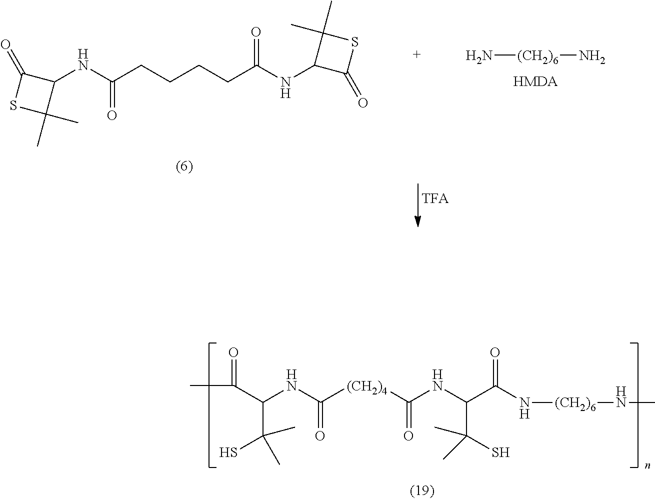

To a stirred solution of N¹,N⁶-bis(2,2-dimethyl-4-oxothietan-3-yl)adipamide (Example 2, compound (6)) in THF, an equimolar amount of HMDA in THF was added dropwise. The solution was stirred at 25° C. for 3 days to give copolymer (19) as a cloudy, white suspension. The ¹H NMR (DMSO-d₆) spectrum of copolymer (19) is shown in FIG. 2. ¹H NMR (400 MHz, DMSO-d₆) δ 8.15-7.73 (m, 2H), 4.56-4.37 (m, 1H), 3.19-2.88 (m, 2H), 2.77-2.63 (m, 1H), 2.36-2.04 (m, 2H), 1.56-1.08 (m, 12H). FTIR-ATR (cm⁻¹) vₘₐₓ=1637.

### Example 18: Reaction of compound 6 with an amine-functionalized polydimethylsiloxane to form N1,N6-bis(3-mercapto-3-methyl-1-((3-(1,1,1,3,5,5,7,7,7-nonamethyltetrasiloxan-3-yl)propyl)amino)-1-oxobutan-2-yl)adipamide (21)

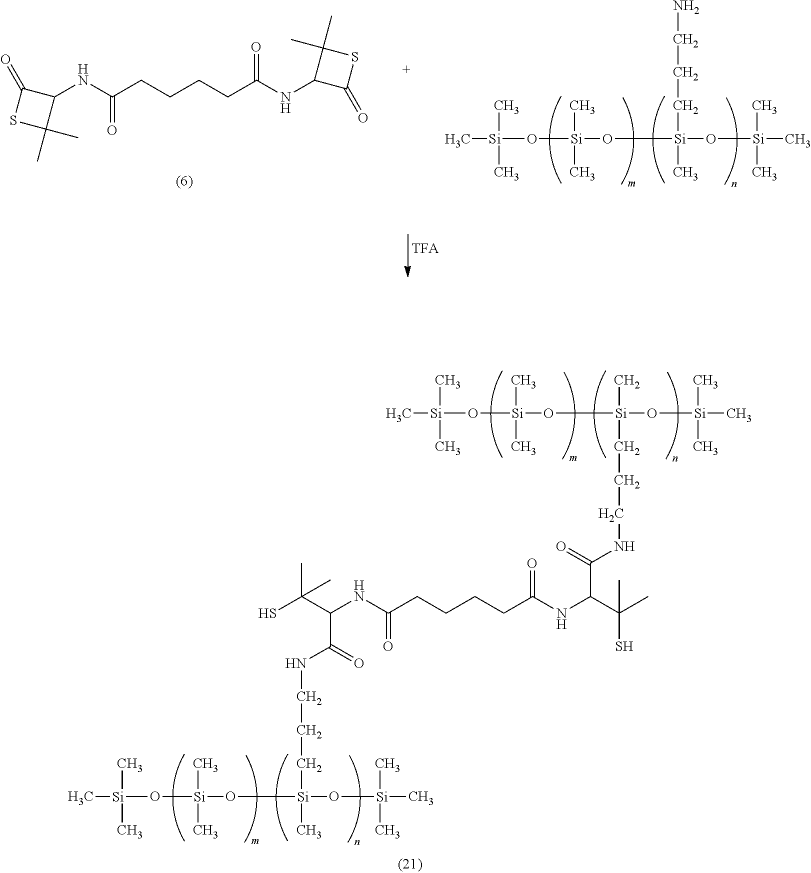

To a stirred solution of 500 mg amine-functionalized PDMS (Gelest AMS-163, described as (6-7% aminopropylmethylsiloxane)-dimethylsiloxane copolymer, 1800-2200 cSt, Mw 50,000 g/mol) in 15 mL CHCl₃ was added 70 mg N¹,N⁶-bis(2,2-dimethyl-4-oxothietan-3-yl)adipamide (Example 2, compound (6)), and the cloudy solution stirred overnight at RT. The following day the cloudy solution had become a clear gel The reactants formed the crosslinked product as a silicone polymer (21), as evidenced by the formation of the clear gel.

### Example A2—Reaction of Compound (6) with 1,3-diamino-2,2-dimethylpropane to form poly(N¹-(1-((2,2-dimethyl-3-(methylamino)propyl)amino)-3-mercapto-3-methyl-1-oxobutan-2-yl)-N6-(3-mercapto-3-methyl-1-(methylamino)-1-oxobutan-2-yl)adipamide) (32)

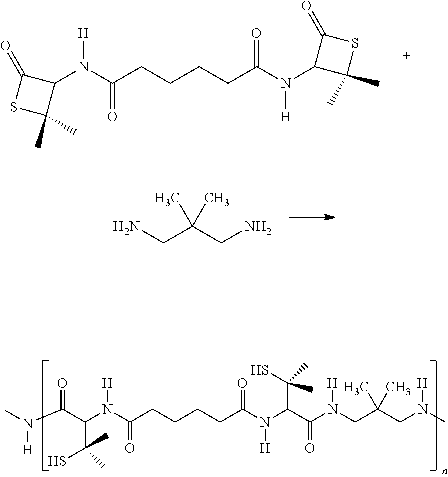

To a 100 mL round bottom flask was added 40 mL of dimethylsulfoxide. The 1,6-bis(thiolactone)adipic amide (5 g) and 1,3-diamino-2,2-dimethylpropane (1.373 g Sigma Aldrich 22690). The solution was stirred for 3 days at 60° C. and the viscous solution was added to 800 mL of water/methanol (4:1) in a dropwise manner. The precipitated polymer was flocculated with 30 g of sodium chloride, filtered and washed with 500 g of deionized water. The polymer precipitate was dried under vacuum. ¹H NMR: δ0.75 (s) δ1.02 (d) δ1.28 (s-broad) δ2.16 (s-broad) δ2.49 (s) δ2.71 (s) δ2.91 (s-broad) δ3.27 (s) δ4.52 (d) δ8.04 (d) GPC-LS Mₙ:16,849 dalton, M_{w}: 43,456 dalton DSC: T_{g} (midpoint) 84.6° C.

The gel permeation chromatography/light scattering plot for this polymer is shown in FIG. 13. Refractive index and light scattering detectors were used and average values for the polymer were obtained as follows: average Mₙ=16,849, average M_{w}=43,456, average M_{z}=80,399 and average M_{w}/Mₙ=2.597.

### Example A3—Reaction of Compound (31) with bis(diamino polypropylene oxide) to form poly(N¹-(2-mercapto-2-methyl-4-oxopentan-3-yl)-N³-(3-mercapto-3-methyl-1-((1-(2-(methylamino)propoxy)propan-2-yl)amino)-1-oxobutan-2-yl)-2,2-dimethylmalonamide) (33)

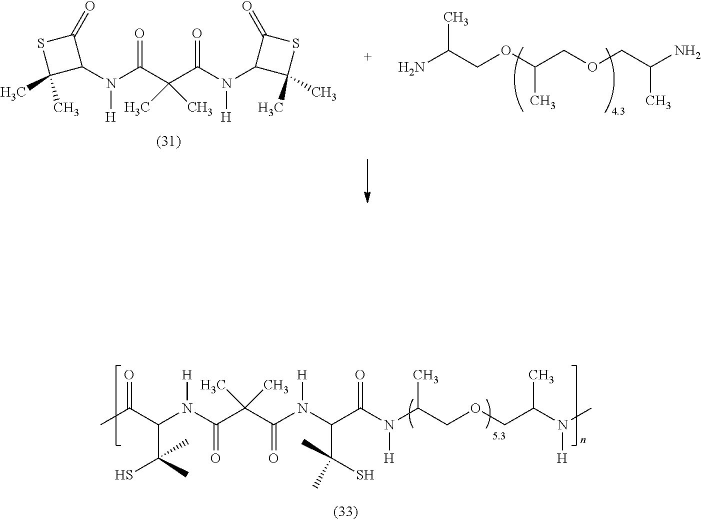

To a round bottom flask was added 2,2′dimethylmalonyl thiolactone (Example A1, compound (31), 5.Og). The monomer was dissolved into dimethyl sulfoxide (40 mL). To the solution was added bis(diamino polypropylene oxide) (M_{w}: 430 g/mol; Sigma Aldrich 406651) and the mixture was stirred at 60° C. for 72 hours. The viscous solution was then added dropwise into deionized water forming a light yellow polymer. The polymer was then re-dissolved into acetone and reprecipitated for a second time into deionized water, and the light yellow polymer (33) dried under vacuum. <Mₙ>:40,890 DSC (midpoint) T₉: 22° C. ¹H NMR (broad): 60.97 (s) δ1.04 (s) δ1.22 (s) δ1.32 (s) δ2.49 (s) δ2.73 (s) δ3.48 (s) δ3.56 (s) δ4.50 (s) δ7.28 (d) δ8.08 (d) δ8.75 (s).

The polymer (33) is referred to in the examples below and in the figures as dimethylmalonylpenicillamine PPO-400 polymer.

### Example A4—Reaction of Compound (31) and bis(diamino polypropylene oxide) with bis(diamino propylene oxide-block polyethylene oxide-block propylene oxide) to form block copolymer P(N¹-(33-mercapto-16,22-bis(2-mercaptopropan-2-yl)-3,6,13,19,19,25,29,33-octamethyl-15,18,20,23,31-pentaoxo-5,8,11,27-tetraoxa-2,14,17,21,24,30-hexaazatetratriacontan-32-y)-N³-(3-mercapto-3-methyl-1-(methylamino)-1-oxobutan-2-yl)-2,2-dimethylmalonamideolyl) (35)

The reaction scheme for formation of this polymer is shown in Scheme 1 below.

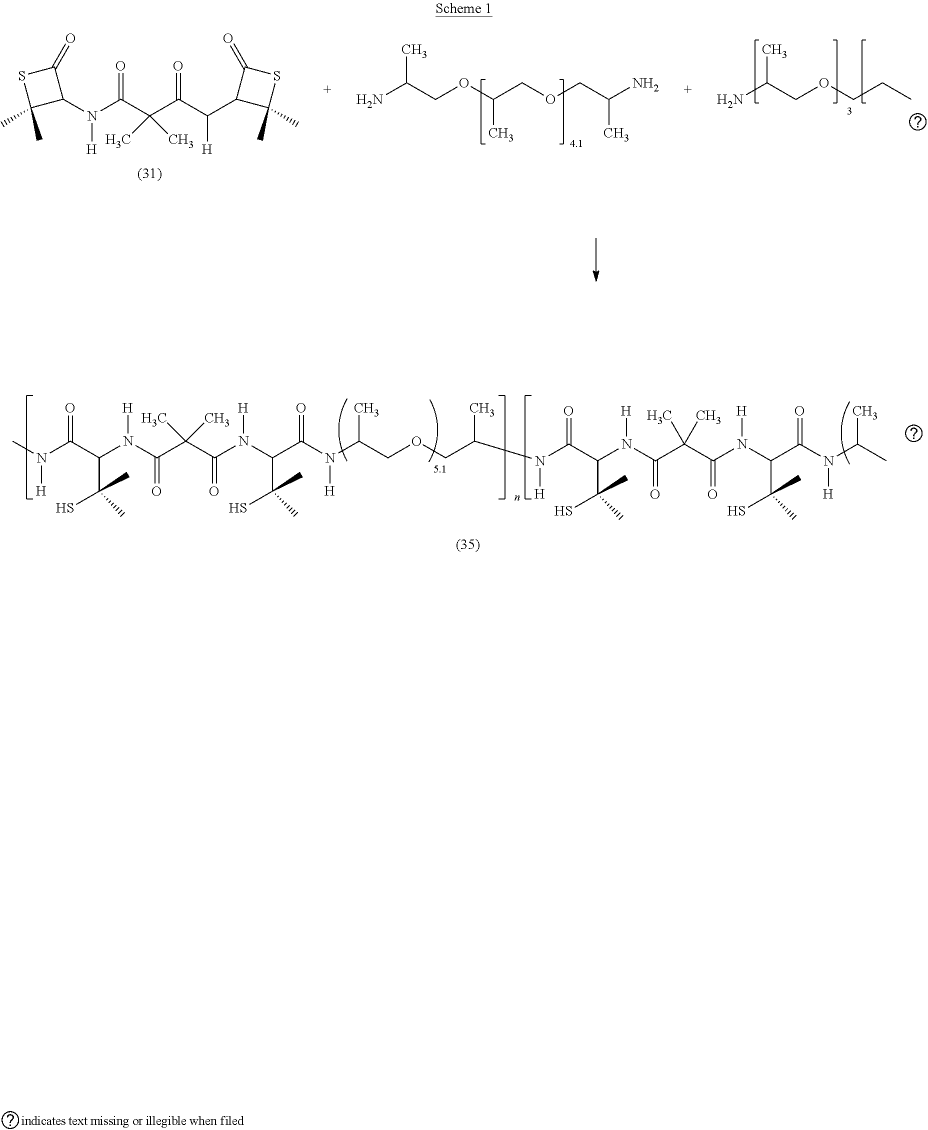

To a round bottom flask was added 2,2′-dimethylmalonyl thiolactone (Example A1, compound (31), 5.000 g) dissolved in 40 mL dimethyl sulfoxide. To the solution was then added bis(diamino polypropylene oxide), M_{w}: 430 g/mol; Sigma Aldrich 406678 (2.790 g) and bis(diamino propylene oxide-block polyethylene oxide-block propylene oxide) M_{w}: 600, Sigma Aldrich 14526 (4.184 g). The solution was stirred at 60° C. for 72 hours forming a viscous solution. The solution was then added dropwise to 800 mL of deionized water. The precipitated polymer was flocculated with 25 grams of sodium chloride and collected by vacuum filtration and washed with an excess of deionized water. The polymer (35) was then dried under vacuum. DSC: Tg (midpoint): 3.6° C. ¹H NMR: δ1.08 61.16 (d-broad) δ1.46 (d-broad) δ2.56 (s) δ2.84 (s) δ3.46 (t-broad) δ4.48 (t-broad) δ4.61 (s) δ7.5 (d-broad) δ8.19 (d-broad).

The polymer is cross linked and therefore has an infinite molecular weight.

### Conversion of “Thiol/—SH” Chemical Entities to “S-Nitrosothiol/—SNO” Chemical Entities

### Example 19: S-nitrosothiol derivatization of compound 14 to form N¹,N⁶-bis(1-(benzylamino)-3-mercapto-3-methyl-1-oxobutan-2-yl)adipamide (16)

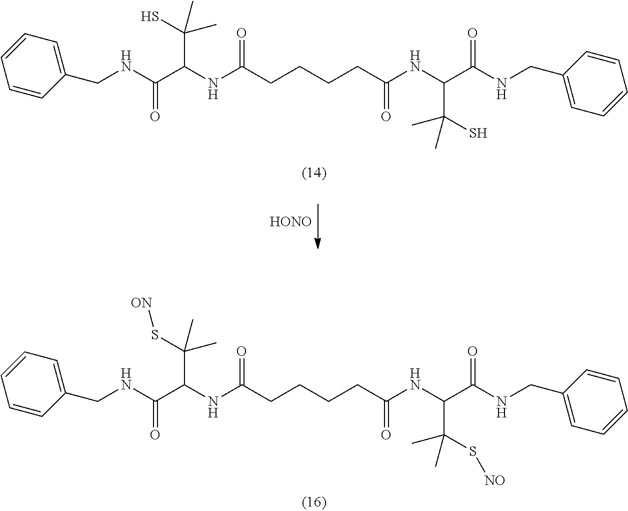

Approximately 200 mg of compound (14) (Example 9) was dissolved in 10 mL of MeOH, followed by 10 mL of 1 M HCl, and the resulting suspension cooled to 0° C. in an ice bath. Then, 2 mL of conc. H₂SO₄ was added, and the suspension again cooled to 0° C. in the ice bath. Sodium nitrite (3 eq. per thiol of compound (14)) was dissolved in a few milliliters of water (forming nitrous acid (HONO) in situ) and added all at once. 10 mL of DCM was added, and the ice bath removed, and the suspension was vigorously stirred in the dark for 1.5 h. The resulting emulsion was then poured into a separatory funnel, and the bright green DCM layer was poured over Na₂SO₄ and placed in a refrigerator (5° C.) for 30 minutes. The DCM layer was filtered, and the DCM solvent was rotary evaporated under reduced pressure to give compound (16) as a green solid. ¹H NMR (400 MHz, CDCl₃) δ 8.25 (s, 2H), 7.87 (d, 2H, J=9.53 Hz), 7.19-7.08 (m, 6H), 6.97-6.90 (m, 4H), 5.68 (d, 2H, J=10.04 Hz), 4.07 (dd, 2H, J=14.92, 5.94 Hz), 3.70 (dd, 2H, J=14.63, 3.62 Hz), 2.35 (m, 4H), 2.05 (s, 6H), 2.02 (s, 6H), 1.76-1.35 (m, 4H). FTIR-ATR (cm¹) vₘₐₓ=1639.

### Example 20: S-nitrosothiol derivatization of compound 15 to form S,S′-((adipoylbis(azanediyl))bis(2-methyl-4-oxo-4-(phenylamino)butane-3,2-diyl)) dinitrothioite (17)

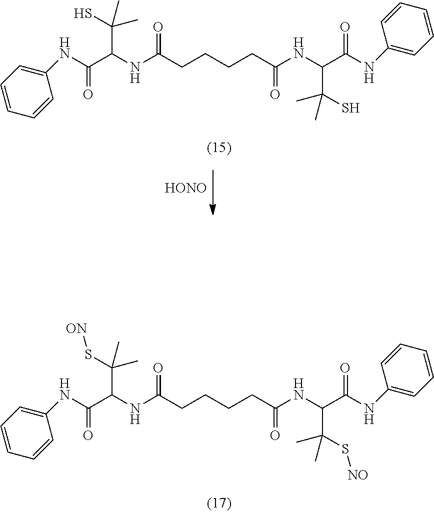

Approximately 200 mg of compound (15) (Example 10) was dissolved in 10 mL of MeOH, followed by 10 mL of 1 M HCl, and the resulting suspension cooled to 0° C. in an ice bath. Then, 2 mL of conc. H₂SO₄ was added, and the suspension again cooled to 0° C. in the ice bath. Sodium nitrite (3 eq. per thiol of compound (15)) was dissolved in a few milliliters of water (forming nitrous acid (HONO) in situ) and added all at once. 10 mL of DCM was added, and the ice bath removed, and the suspension was vigorously stirred in the dark for 1.5 h. The resulting emulsion was then poured into a separatory funnel and the bright green DCM layer was poured over Na₂SO₄ and placed in a refrigerator (5° C.) for 30 minutes. The DCM layer was filtered, and the DCM solvent was rotary evaporated under reduced pressure to give compound (17) as a green solid. ¹H NMR (400 MHz, DMSO-d₆) δ 10.47 (s, 2H), 8.43 (d, 2H, J=9.59 Hz), 7.59 (m, 4H), 7.31 (m, 4H), 7.08 (m, 2H), 7.43 (d, 2H, J=9.59 Hz,), 2.18 (m, 4H), 2.03 (m, 6H), 1.98 (m, 6H), 1.39 (m, 4H). FTIR-ATR (cm⁻¹) vₘₐₓ=1496.

### Example 21: S-nitrosothiol Derivatization of Compound (8c) to form S,S′-((isophthaloylbis(azanediyl))bis(2-methyl-4-morpholino-4-oxobutane-3,2-diyl)) dinitrothioite (8d)

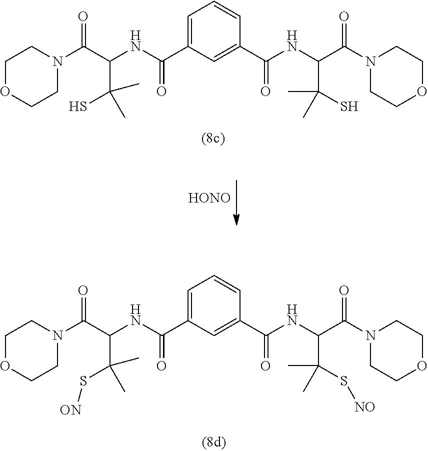

Approximately 200 mg of compound (8c) (Example 14) was dissolved in 10 mL of MeOH, followed by 10 mL of 1 M HCl, and the resulting suspension cooled to 0° C. in an ice bath. Then, 2 mL of conc. H₂SO₄ was added, and the suspension again cooled to 0° C. in the ice bath. Sodium nitrite (3 eq. per thiol of compound (8c)) was dissolved in a few milliliters of water (forming nitrous acid (HONO) in situ) and added all at once. 10 mL of DCM was added, and the ice bath removed, and the suspension was vigorously stirred in the dark for 1.5 h. The resulting emulsion was then poured into a separatory funnel and the bright green DCM layer was poured over Na₂SO₄ and placed in a refrigerator (5° C.) for 30 minutes. The DCM layer was filtered, and the DCM solvent was rotary evaporated under reduced pressure to give compound (8d) as a green solid. ¹H NMR (400 MHz, DMSO-d₆) δ 9.10 (d, 2H, J=9.19 Hz), 8.16 (t. 1H, J=1.54 Hz), 7.94 (dd, 2H, J=7.76, 1.71 Hz), 7.51 (t, 1H, J=7.76 Hz), 5.86, (d, 2H, J=9.21 Hz), 3.63-3.42, (m, 16H), 2.12 (s, 6H), 2.04 (s, 6H). FTIR-ATR (cm⁻¹) vₘₐₓ=1631.

### Example 22: S-nitrosothiol Derivatization of Compound 10a to form S,S′,S″-(((benzene-1,3,5-tricarbonyl)tris(azanediyl))tris(4-(benzylamino)-2-methyl-4-oxobutane-3,2-diyl)) trinitrothioite (18)

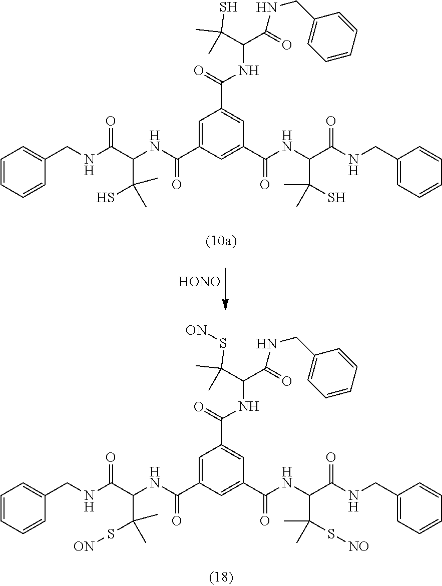

Approximately 200 mg of compound (10a) (Example 15) was dissolved in 10 mL of MeOH, followed by 10 mL of 1 M HCl, and the resulting suspension cooled to 0° C. in an ice bath. Then, 2 mL of conc. H₂SO₄ was added, and the suspension again cooled to 0° C. in the ice bath. Sodium nitrite (3 eq. per thiol of compound (10a)) was dissolved in a few milliliters of water (forming nitrous acid (HONO) in situ) and added all at once. 10 mL of DCM was added, and the ice bath removed, and the suspension was vigorously stirred in the dark for 1.5 h. The resulting emulsion was then poured into a separatory funnel and the bright green DCM layer was poured over Na₂SO₄ and placed in a refrigerator (5° C.) for 30 minutes. The DCM layer was filtered, and the DCM solvent was rotary evaporated under reduced pressure to give compound (18) as a green solid. ¹H NMR (400 MHz, CDCl₃) δ 8.21 (s, 3H), 7.92 (d, 3H, J=8.76 Hz), 7.46 (t, 3H, J=4.88 Hz), 7.30-7.19 (m, 12H), 4.80 (d, 3H, J=8.81 Hz), 4.60 (dd, 3H, J=14.64, 6.13 Hz), 4.28 (dd, 3H, J=14.64, 4.63 Hz), 2.50 (s, 3H), 1.60 (s, 9H), 1.41 (s, 9H). FTIR-ATR (cm⁻¹) vₘₐₓ=1496.

### Example 23: S-nitrosothiol derivatization of copolymer 19 to form S-(2-methyl-3-(6-((2-methyl-2-(nitrosothio)-4-oxopentan-3-yl)amino)-6-oxohexanamido)-4-((6-(methylamino)hexyl)amino)-4-oxobutan-2-yl) nitrothioite (20)

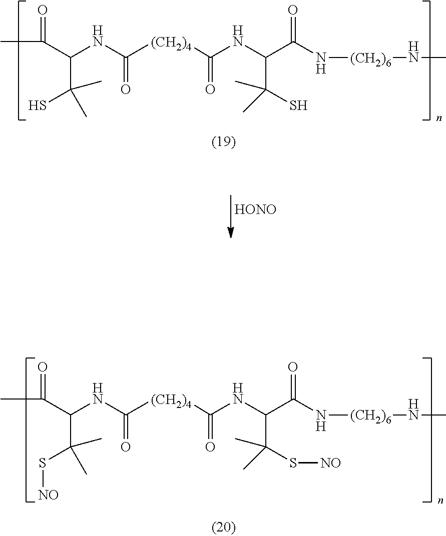

To approximately 200 mg of copolymer (19) (Example 17) suspended in 10 mL of THF, 10 mL of 1 M HCl was added, and the suspension cooled to 0° C. in an ice bath. Then, 2 mL of conc. H₂SO₄ was added, and the suspension again cooled to 0° C. in the ice bath. Sodium nitrite (3 eq. per thiol of compound (19)) was dissolved in a few milliliters of water (forming nitrous acid (HONO) in situ) and added all at once. The ice bath was removed, and the suspension stirred in the dark for 3 h. The resulting green suspension was then collected on a frit and washed with water and dried overnight under vacuum to give copolymer (20). ¹H NMR (400 MHz, DMSO-d₆) δ 8.46-8.14 (m, 2H), 5.24-5.09 (m, 1H), 3.23-2.86 (m, 2H), 2.37-2.02 (m, 2H), 2.02-1.80 (m, 6H), 1.22-1.07 (m, 6H). FTIR-ATR (cm⁻¹) vₘₐₓ=1641. The FTIR spectra of both copolymers (19) and (20) is overlayed in FIG. 3.

### Example 24: S-nitrosothiol Derivatization of Silicone Polymer (21) to form S,S′-((adipoylbis(azanediyl))bis(2-methyl-4-((3-(1,1,1,3,5,5,7,7,7-nonamethyltetrasiloxan-3-yl)propyl)amino)-4-oxobutane-3,2-diyl)) dinitrothioite (22)

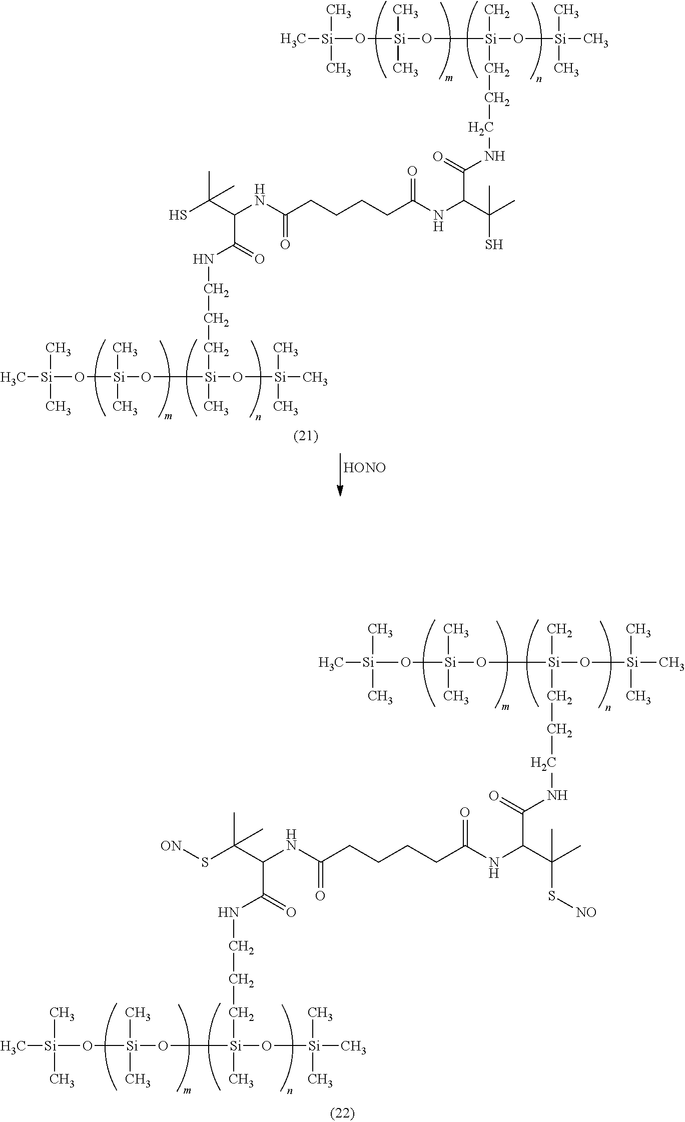

Silicone polymer (21) as prepared according to Example 18 was converted to the S-nitrosothiol silicone polymer (22) by adding 5 mL of glacial acetic acid followed by tert-butyl nitrite (2 eq. per eq. silicone (21)), and the gel began to turn green immediately. The green gel was periodically shaken vigorously over the course of an hour while being kept in the dark, to yield S-nitrosothiol silicone polymer (22), which was observed to release nitric oxide, using Evaluation Method Ai.

### Example A5: S-nitrosothiol derivatization of Poly(N¹-(2-mercapto-2-methyl-4-oxopentan-3-yl)-N³-(3-mercapto-3-methyl-1-((1-(2-(methylamino)propoxy)propan-2-yl)amino)-1-oxobutan-2-yl)-2,2-dimethylmalonamide) (33) to form nitrosylated polymer (37)

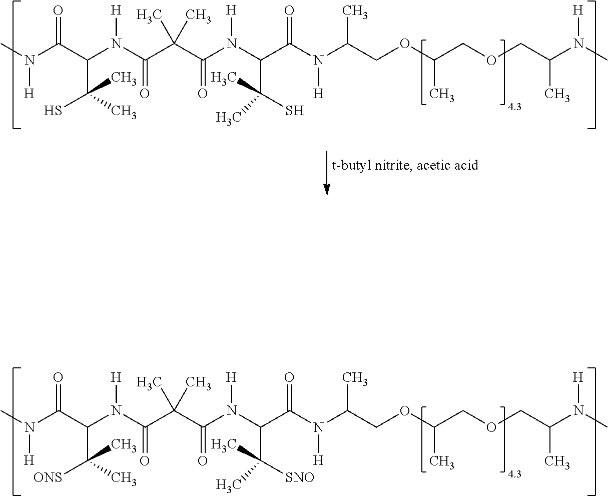

The dimethylmalonyl penicillamine PPO (400) polymer (33) of Example A3 (2 g) was dissolved in acetone (40 mL) forming a light yellow solution. To the solution was added tert-butylnitrite (Sigma Aldrich 235385, 299 μL) with acetic acid (500 μL). The solution was stirred overnight with the roundbottom flask wrapped in aluminum foil to protect from light, with the solution changing from yellow to dark green characteristic of the formation of a S-nitrosothiol, described in Hopkins and Frost (2020). The solution was then stirred for 12 hours at room temperature after which the acetone was then removed under vacuum to leave the nitrosylated polymer (37). T_{g}: (midpoint) 21.2° C. FT-IR Loss of —SH stretch 2551 cm⁻¹, development of S—NO stretch: 1553 cm⁻¹.

The FT-IR spectrum (FIG. 14) showed the loss of —SH stretch at 2551 cm⁻¹ and the development of the S—NO stretch: 1553 cm⁻¹. In FIG. 14, the dashed line is the spectrum for the non-nitrosylated form and the solid line is the spectrum for the nitrosylated form.

A TGA (FIG. 15) was taken to determine the extent of nitrosylation as shown in Evaluation method Aii above. In FIG. 15, the solid line represents the non-nitrosylated form and the dashed line represents the nitrosylated form. The weight fraction of nitrosylation is 4.868 wt % or 68% fractional conversion.

Samples of the nitrosylated polymer of PPO-400 dimethylmalonyl penicillamine (33) dissolved in acetone at 5 wt % were then diluted to 2.5 wt % with acetone. The polymers were then coated by drop-coating onto a glass substrate and tested for NO elution using the Griess Reagent NO elution test method (Evaluation Method Aiii above). FIG. 16 shows the elution of NO from the polymer over a period of 27 days (648 hours). It can be seen that the plot has a substantially constant gradient, indicating first order release of NO from the polymer.

### Preparation of Surface Coatings of S-Nitrosothiol Chemical Entities

### Example 25: Preparation of NO-Releasing Coating on ePTFE Films

In separate experiments, chloroform solutions of S-nitrosothiol-containing compound 16 (Example 19) and compound 17 (Example 20) were deposited dropwise onto ePTFE films secured on embroidery hoops, allowing the solvents to evaporate as the solutions were deposited. Once the solvents had evaporated and the resulting coatings were dry, 10 mL of a PBS buffer solution comprising 10 mM phosphate buffered saline with 100 μM EDTA, was poured over the individual coated films in round, polyethylene containers with screw-cap lids and placed in a 37° C. water bath. The PBS buffer solutions were tested periodically for NO release according to Evaluation Method Ai. The NO release profile for the film coated with compound 16 is shown in FIG. 4, and for the film coated with compound 17, in FIG. 5.

The results shown in FIG. 4 demonstrate immediate release of NO from the coating comprising compound 16, reaching steady state of NO release at 5 days. The results shown in FIG. 5 demonstrate immediate release of NO from the coating comprising compound 17, reaching a steady state concentration of nitrite anion at 7 days. The results shown in FIGS. 4 and 5 demonstrate that coatings comprising compounds of the present disclosure are effective at immediately releasing NO from the coatings deposited on ePTFE films, for several days.

### Example 26: Preparation of NO-Releasing Coating on ePTFE Films with Additional Fluoropolymer Coating

In separate experiments, acetone solutions of 50 mg of the S-nitrosothiol-containing compound 16 (Example 19), compound 18 (Example 22), and copolymer 20 (Example 23) were added to individual mixtures of 200 mg of VDF-HFP fluoropolymer in 10 mL of acetone. The green mixtures were then poured onto 3-inch disks of ePTFE films secured on embroidery hoops, and the solvents evaporated under a stream of argon. Once the resulting coatings were dry, 10 mL of the PBS buffer solutions were individually poured over the coated films in round, polyethylene containers with screw-cap lids and placed in a 37° C. water bath. The PBS buffer solutions were tested periodically for NO release according to Evaluation Method Ai. The NO release profile for the film coated with VDF-HFP fluoropolymer and compound 16 is shown in FIG. 6, the NO release profile for the film coated with VDF-HFP fluoropolymer and compound 18 is shown in FIG. 7, and the NO release profile for the film coated with VDF-HFP fluoropolymer and copolymer 20 is shown in FIG. 11.

The results shown in FIG. 6 demonstrate 2 day delayed release of NO from the coating comprising VDF-HFP fluoropolymer and compound 16, reaching a steady state concentration of nitrite anion at 8 days. The release profile is similar to the release profile of the coating comprising compound 16 when applied to the ePTFE film (FIG. 4), but with an initial delayed release of 2 days, demonstrating the inclusion of the VDF-HFP fluoropolymer was effective at delaying the onset of release.

The results shown in FIG. 7 demonstrate 2 day delayed release of NO from the coating comprising VDF-HFP fluoropolymer and compound 18, reaching a steady state concentration of nitrite anion at 12 days. The results demonstrate the inclusion of the VDF-HFP fluoropolymer was effective at delaying the onset of release.

The results shown in FIG. 11 demonstrate 6 day delayed release of NO from the coating comprising VDF-HFP fluoropolymer compound 20, reaching a steady state concentration of nitrite anion at 9 days. The results demonstrate the inclusion of the VDF-HFP fluoropolymer was effective at delaying the onset of release.

When compared to the release profiles of Example 25, which demonstrated immediate release of NO from their respective coated compounds, the release profiles of Example 26 demonstrated delayed release, demonstrating the inclusion of a fluoropolymer in the coating was effective at delaying the onset of release.

### Example 27: Preparation of NO-Releasing Coating on Stainless Steel with Additional Fluoropolymer

In separate experiments, an acetone solution comprising 50 mg of the S-nitrosothiol-containing compound 18 (Example 22) was added to a solution of 200 mg of VDF-HFP fluoropolymer in 10 mL of acetone. The green solution was then poured onto a stainless steel foil. Once the solvent was evaporated and the coating was dry, 10 mL of PBS buffer solution was poured over the coated foil in a polyethylene container with a screw-cap lid and placed in a 37° C. water bath. The PBS buffer solution was tested periodically for NO release according to Evaluation Method Ai. The NO release profile for the foil coated with VDF-HFP fluoropolymer and compound 18 is shown in FIG. 8.

The results shown in FIG. 8 demonstrate 9 day delayed release of NO from the coating comprising VDF-HFP fluoropolymer and compound 18, reaching a steady state concentration of nitrite anion at 14 days. The results demonstrate the inclusion of the VDF-HFP fluoropolymer was effective at delaying the onset of release of NO from a stainless steel foil.

### Example 28: Preparation of NO-Releasing Coating on a Pebax® Nylon Film with Additional Fluoropolymer

In separate experiments, an acetone solution of 50 mg of the S-nitrosothiol-containing compound 18 (Example 22), was added to a solution of 200 mg of VDF-HFP fluoropolymer in 10 mL of acetone. The green solution was then poured onto Pebax® nylon film. The acetone was evaporated to form a coating; once the coating was dry, 10 mL of PBS buffer solution was poured over the coated film in a polyethylene container with a screw-cap lid and placed in a 37° C. water bath. The PBS buffer solution was tested periodically for NO release according to Evaluation Method Ai. The NO release profile for the nylon coated with VDF-HFP fluoropolymer and compound 18 is shown in FIG. 9.

The results shown in FIG. 9 demonstrate 4 day delayed release of NO from the coating comprising VDF-HFP fluoropolymer and compound 18, reaching a steady state concentration of nitrite anion at 13 days. The results demonstrate the inclusion of the VDF-HFP fluoropolymer was effective at delaying the onset of release of NO from a Pebax® nylon film, similar to the results for a coating comprising VDF-HFP fluoropolymer and compound 18 when applied onto an ePTFE film (FIG. 7), and when applied onto a stainless steel foil (FIG. 8).

### Example 29: Preparation of NO-Releasing Coating on an ePE Film with Additional Fluoropolymer

In separate experiments, an acetone solution of 50 mg of the S-nitrosothiol-containing of compound 18 (Example 22) was added to a solution of 200 mg of VDF-HFP fluoropolymer in 10 mL of acetone. The green solution was then poured onto an ePE film. The acetone was evaporated to form a coating; once the coating was dry, 10 mL of PBS buffer solution was poured over the coated film in a polyethylene container with a screw-cap lid and placed in a 37° C. water bath. The PBS buffer solution was tested periodically for NO release according to Evaluation Method Ai. The NO release profile for the ePE coated with VDF-HFP fluoropolymer and compound 18 is shown in FIG. 10.

The results shown in FIG. 10 demonstrate 2 day delayed release of NO from the coating comprising VDF-HFP fluoropolymer and compound 18, reaching a steady state concentration of nitrite anion at 15 days. The results demonstrate the inclusion of the VDF-HFP fluoropolymer was effective at delaying the onset of release of NO from an ePE film, similar to the results for a coating comprising VDF-HFP fluoropolymer and compound 18 when applied onto an ePTFE film (FIG. 7), when applied onto a stainless steel foil (FIG. 8), and when applied onto a Pebax® nylon film (FIG. 9).

### Example 30: Preparation of NO-Releasing Silicone Coating on ePTFE

The S-nitrosothiol silicone polymer (22) prepared according to Example 24 was poured onto a 3-inch disk of ePTFE film secured on an embroidery hoop, and the solvent evaporated under a stream of argon. Once the solvent was evaporated and the resulting coating allowed to dry, 10 mL of PBS buffer solution was poured over the coated film in a round, polyethylene container with screw-cap lid and placed in a 37° C. water bath. The PBS buffer solution was tested periodically for NO release according to Evaluation Method Ai. The NO release profile for the film coated with polymer 22 is shown in FIG. 12.

The results shown in FIG. 12 demonstrate immediate first release of NO from the coating comprising VDF-HFP fluoropolymer and compound 18, reaching a steady state concentration of nitrite anion at 7 days.

### Example A6—Coating Vascular Graft and Stent Graft with Nitrosylated Polymer (37) of Example A5

To a stainless steel 8 mm mandrel was added a 8 mmm×50 mm vascular graft or stent graft. A Sonotek DES4000 spray coater was fitted with a IVEK 3500001-46 nozzle. The nitrosylated 2,2′dimethylmalonylpenacillamine bis(diamino polypropylene oxide) (37) was dissolved into acetone (0.5 wt %) and a 20 mL syringe was filled with the nitrosylated polymer solution. The stent and vascular graft were both spray coated on the last 1 cm with 2.0 mL nitrosylated polymer solution at 0.25 mL/min using a nitrogen carrier gas at 3.5 psi while the stent or graft was rotated at 100 rpm's. The coating was then dried at 23° C. for 8 minutes, before coating the end of the stent or graft a second time with an additional 2 mL of nitrosylated polymer solution. The coating was then dried an additional 8 minutes at room temperature resulting in a green polymer coating.

FIG. 17 is a series of scanning electron microscope (SEM) images of the surfaces of the coated and uncoated vascular grafts. FIGS. 17a and 17b are images at 50× magnification of the uncoated and coated grafts respectively and FIGS. 17c and 17d are images at 2000× magnification of the coated and uncoated grafts respectively.

The images show that the polymer coating forms a uniform coating, which is free of defects, on the graft or stent graft.

### Example A7—Adhesion and Brittleness Testing of Coating of Nitrosylated Polymer (37) of Example A5 to Vascular Graft

The sample adhesion was tested using ASTM F1842-15 using a Gardco Cross Hatch/WFT Temper 2 ASTM test kit.

A vascular graft was coated with a 0.5 wt % solution of nitrosylated polymer (37) in acetone. The vascular graft was coated along 125 mm of its length with 10 mL of solution using the procedure described above with a 1 mm/sec translation rate. The coating adhesion was tested per ASTM F1842-15 using a Gardco Cross Hatch/WFT Temper 2 ASTM test kit. The resistance for cracking of the coating was tested by passing the coated stent graft around a stainless steel cone with an outer diameter of 15 mm per ISO 7198. The coating was then evaluated by SEM. The thinner coating has a coating structure similar to the underlying node and fibril structure of the substrate.

FIGS. 18A and 18B are SEM images at 30× and 200× magnification respectively of the vascular graft coated with the nitrosylated polymer (37) after being passed round the 15 mm cone. It can be seen that the coating remains intact and that the surface is more uniform than that of the uncoated graft shown in FIGS. 17A and C.

### In Situ Formation of Chemical Entity Coatings

### Example 31: In Situ Formation of Thiol-Containing Polymer on a Coated Surface

A 3-inch disk of ePTFE film was secured on an embroidery hoop and submersed in a 4% aqueous PEI solution for 15 minutes. The film was rinsed with deionized water, then placed in 800 mL of fresh, deionized water for 15 minutes with stirring. The film was then divided into two samples. The first sample was placed in 50 mL of a methanol solution containing 20 mg of adipoyl-1,6-dipenicillamine dithiolactone (Example 2, compound (6)), resulting in the formation of a crosslinked PEI polymer on the surface of the ePTFE film. The second sample was used as is (i.e., there was an uncross-linked PEI polymer on the surface of the ePTFE film).

Both samples were then stained red using Ponceau S (a dye which stains PEI coatings with a red color) and rinsed in deionized water. Both samples stained red, indicating the deposition of PEI. Then, each stained sample was separately placed in boiling isopropyl alcohol (IPA) for 15 minutes and subsequently rinsed with additional IPA. Boiling IPA effectively removes uncross-linked PEI coatings from a surface, due to such coatings being only physisorbed on the surface, whereas boiling IPA does not remove crosslinked PEI coatings from a surface, due to such coatings being chemically crosslinked on the surface. The second uncross-linked PEI sample was no longer stained red, demonstrating the boiling IPA had effectively removed the uncross-linked (i.e., only physisorbed) PEI from the ePTFE film, whereas the first crosslinked PEI sample remained stained red, indicating that the PEI was resistant to removal from the ePTFE surface by boiling IPA, demonstrating the PEI coating was chemically crosslinked on the surface, as a crosslinked PEI coating, by reaction of the PEI with adipoyl-1,6-dipenicillamine dithiolactone (Example 2, compound (6)).

### Analysis of Chemical Entities and Coatings

### Example A8: Mass Spectrometry Analysis of Chemical Entities and Coatings of the Invention

Mass spectrometry can be used to determine whether a chemical entity, for example a chemical entity forming a coating, has been formed by reaction of components B and C as defined herein. When the chemical entity or coating formed from the chemical entity is soluble in a convenient solvent, HPLC-mass spectrometry may be used. In cases where a chemical entity or coating is not soluble in a convenient solvent, it is more appropriate to use MALDI and/or ToF-SIMS.

HPLC and MS analysis were carried out using the following instruments and conditions.

### Agilent 1260 HPLC Conditions:

Column: 2.1 mm×100 mm, 3.5 m, Zorbax Eclipse XDB-C8
Column Oven: 50° C.
Injection: 3 μL
Solvent A: Aqueous 10 mM Ammonium Acetate
Solvent B: Acetonitrile
Flow (ml/min): 0.4

| Gradient: | Time(min) | % B |
|---|---|---|
| | 0.00 | 20.0 |
| | 0.50 | 20.0 |
| | 1.00 | 25.0 |
| | 2.00 | 55.0 |
| | 3.00 | 80.0 |
| | 4.00 | 90.0 |
| | 5.00 | 95.0 |
| | 7.00 | 98.0 |
| Stop time | 10 minutes | |
| Post time | 6 minutes | |

### Agilent 6540 Q-TOF-MS Conditions

Agilent JetStream electrospray ionization positive mode
Positive Calibration 2 GHz Extended Dynamic Range
Data Acquisition: Profile; MS Abs. Threshold: 200; MS Rel. Threshold 0.01%
Gas Temp.: 320° C., Flow: 11.0 L/min., Nebulizer: 45 psi
Sheath gas flow: 12 L/min; Sheath gas temp.: 350° C.
Fragmentor: 150 V Skimmer 60 V Octapole RFV: 750 V
Capillary: 3000 V; Scan Rate: 1.5 scans/s; Scan Range: m/z 80-1000
Nozzle voltage=0 V
Reference Correction: Enabled, Reference masses: m/z 121.00509, 922.0098
0.1% acetic acid (aq.) added post column, 0.1 mL/min

### Sample Preparation

Samples were prepared by mass and dilution in acetonitrile to a concentration of 1 μg/mL.

ESI spectra of Compound (14) and Compound (38) were determined. Compound (38) was formed using a similar method to compound (14) by reaction of hexamethyldiamine and two equivalents of N-acetyl penicillamine thiolactone as shown in the scheme below:

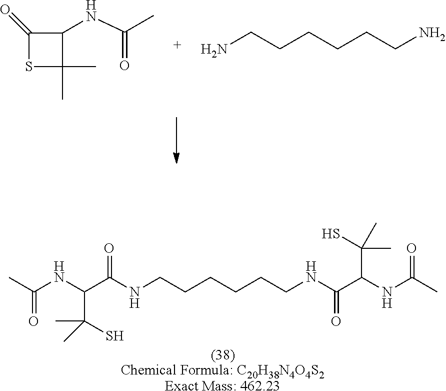 [SMILES: CC(=O)NC1C(=O)SC1(C)C ; NCCCCCCN ; CC(=O)NC(C(=O)NCCCCCCNC(=O)C(NC(C)=O)C(C)(C)S)C(C)(C)S ; "(38)Chemical Formula: C20H38N4O4S2Exact Mass: 462.23"]

Compound (14) is an example of a compound of Embodiment 3 of FIG. 1A, while Compound (38) is an example of a compound of Embodiment 2 of FIG. 1A.

MS analysis of both samples under the conditions set out above resulted in fragmentation into the thiolactone and amine starting materials (reverse polymerization). It was therefore possible to identify molecular fragments that indicate the presence of structural units derived from component B given by Formula (I).

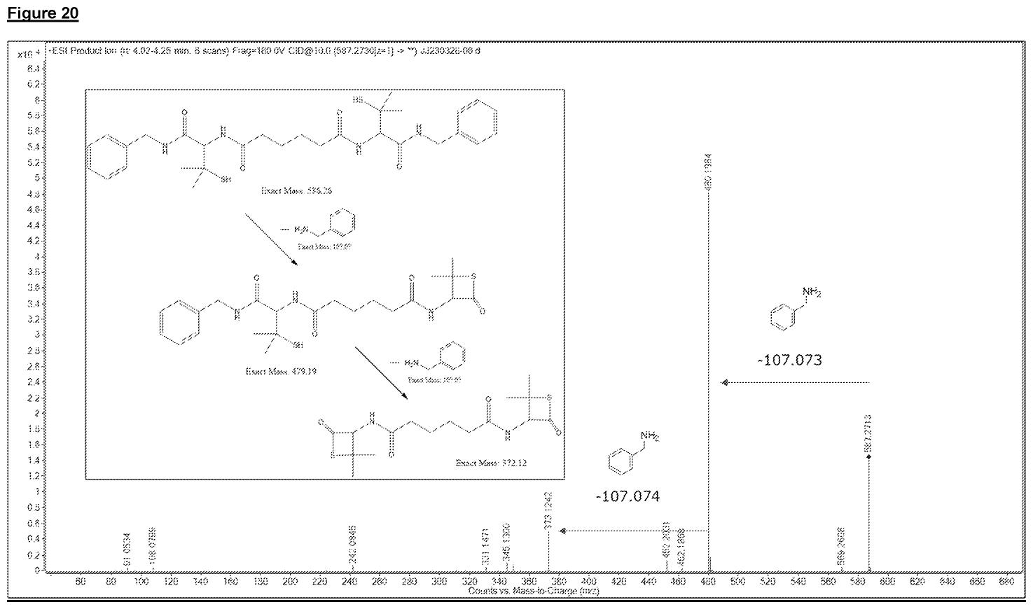

FIG. 19 is the ESI mass spectrum of Compound (14), and shows the M+H⁺ and M+Na⁺ ion peaks. FIG. 20 is a mass spectrum of Compound (14) carried out under Q-ToF-MS/MS conditions as described above such that fragmentation of the compound occurs. The spectrum has a peak at 587.2713 representing M+H⁺ for the unfragmented compound (14) and peaks at 480.1984 and 373.1242, representing respectively fragmented compound (14) from which one or both of the amine fragments of mass 107.07 have been extracted. There is also a peak at 108.0799 which represents M+H⁺ of the amine fragment.

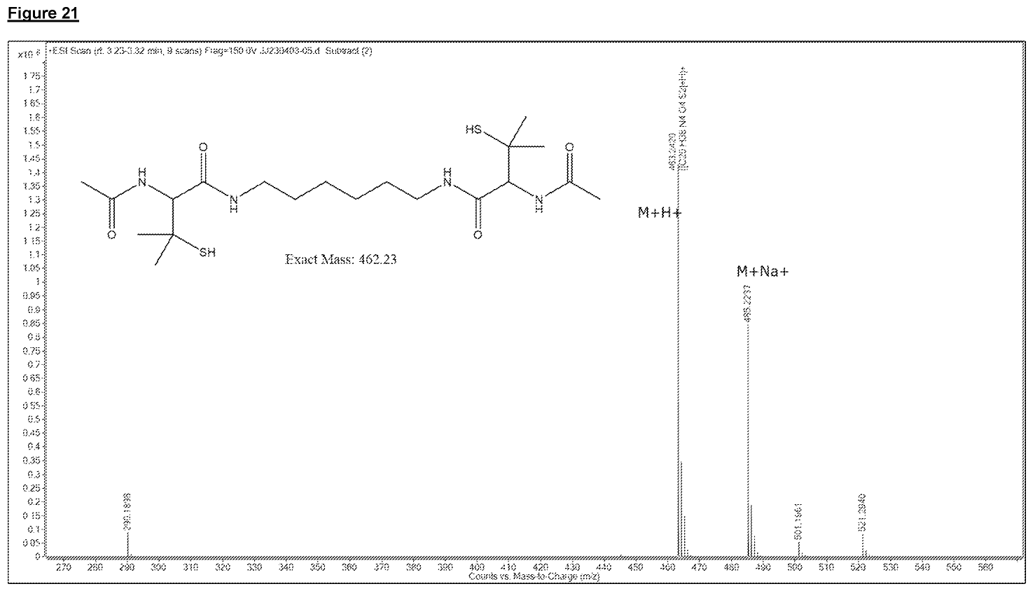

FIG. 21 is an ESI mass spectrum of Compound 38 showing peaks at 463.2420 and 485.2337 representing M+H⁺ and M+Na⁺ respectively.

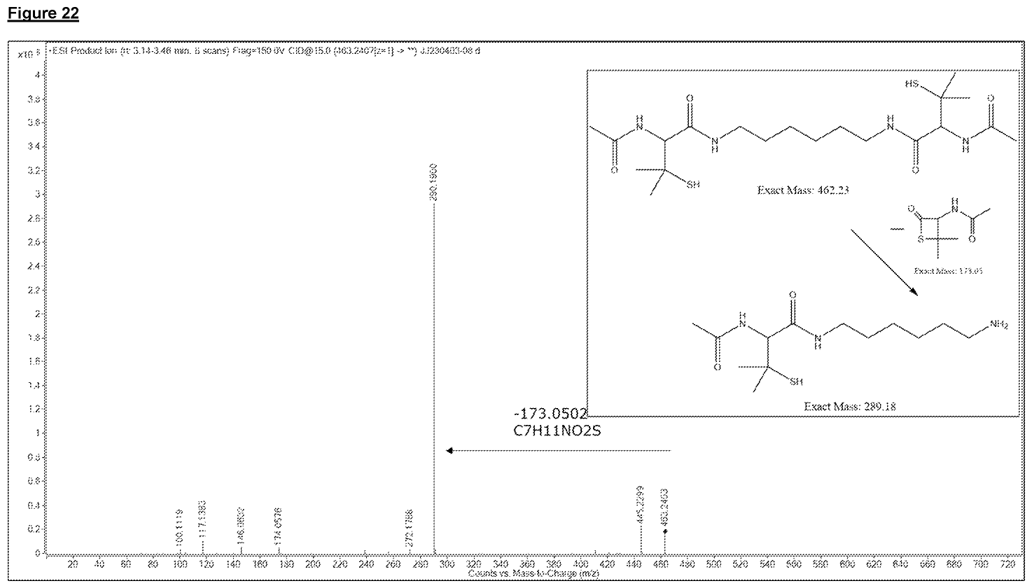

FIG. 22 is a Q-ToF mass spectrum of Compound 38 following fragmentation and has a peak at 463.2403, which represents M+H⁺ of the unfragmented compound and a peak at 290.1900 representing M+H⁺ of a fragment which has lost one thiolactone fragment of mass 173.05.

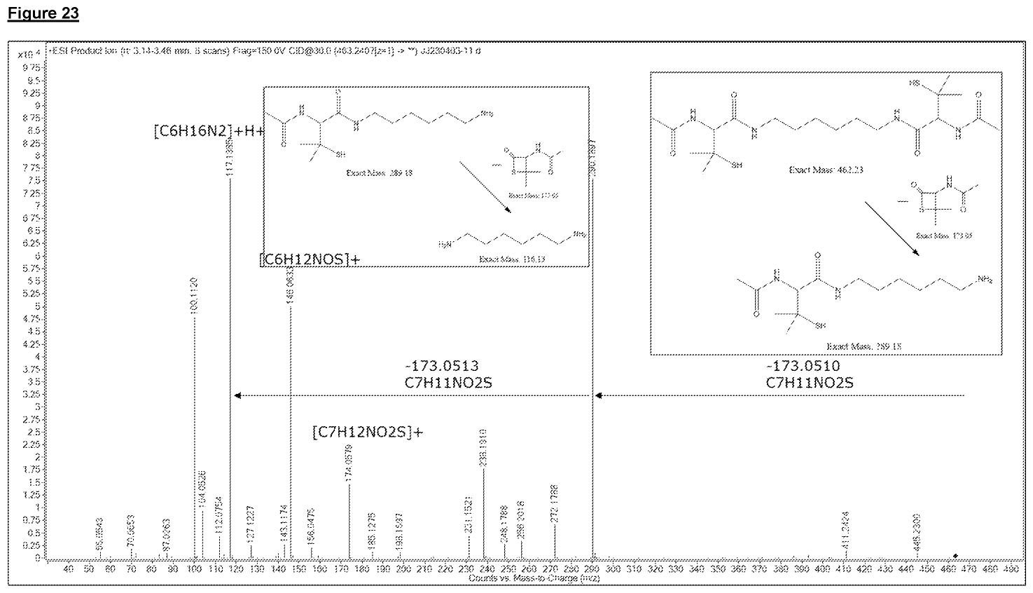

FIG. 23 is an ESI mass spectrum of Compound (38) following fragmentation under harsher conditions and shows peaks at 290.1897, 174.057 and 117.13 corresponding to M+H⁺ for a fragment which has lost one thiolactone fragment, for the thiolactone fragment and for the remaining amine fragment after loss of both thiolactone fragments.

A comparison of FIGS. 20, 22 and 23 therefore demonstrates that it is possible to show that a material or coating contains penicillamine-type moieties that are bonded through the N-terminus to a central core moiety A as for Compound (14). Furthermore, as demonstrated in FIGS. 20, 22 and 23, this analysis distinguishes between structures of the type of Compound (14) and those similar to Compound (38), which consist of exclusively C-bonded penicillamine-type moieties such as N-acetyl penicillamine.

Chemical entities and coatings that may be examined by MS methods are likely to exist as the nitrosothiol form. Nitrosothiols can be converted to thiols prior to analysis by treating the chemical entity or coating by exposing it to a solution containing an excess of a low molecular weight thiol.

As shown in Scheme 2, the NO group will be transferred to the thiol in solution, leaving behind thiols in the chemical entity or coating under investigation.

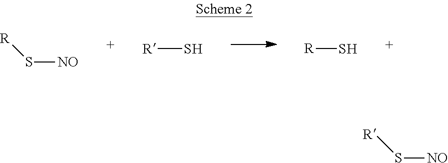

MS analysis of the resulting thiol-containing material will then reveal the components of construction as described above.

In cases of materials or coatings that cannot be taken into a solution for analysis, the use of analytical mass spectrometry techniques such as MALDI and/or ToF-SIMS could be used to analyze a solid material or surface and acquire similar pertinent structural information.

### REFERENCES

Altenbruun et al. Pure Appl. Chem. 2009, 81(2), pp 273-284
Calvete, Proceedings of the Society for Experimental Biology and Medicine 1995, 208(4):346-360
Chen et al. Chem. Sci. 2018, 9, 1982-1988
Cooke and Losordo, Circulation 2002, 105(18):2133-2135
Frost et al. Biomaterials 2005, 26(14):1685-1693
Hopkins et al. Bioengineering 2018, 5(3), 72
Hopkins and Frost Bioengineering 2020, 7(1), 9Ignarro et al. Proc. Nat. Acad. Sci. USA 1993, 90, 8103-8107
Martens et al. Synthesis, 1991, 497-498
Matthys and Bult, Mediators Inflamm. 1997, 6(1):3-21
Schairer et al. Virulence 2012, 3(3):271-279
Xue et al. Int. J. Mol. Sci. 2018, 19, pp 3805

### Miscellaneous

The present disclosure embraces all combinations of indicated groups and embodiments of groups recited above.

All patents and patent applications referred to herein are incorporated by reference in their entirety.

Percentage values given in this specification are based on weight unless otherwise indicated.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

1.-57. (canceled)

58. A chemical entity formed by reaction of components B and C, wherein component B is a compound of Formula (I): 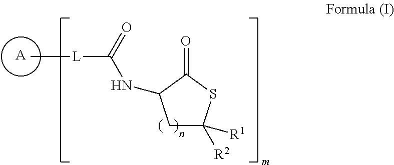
wherein,
A is a core moiety;
L is an optional linker;
R¹ and R² are independently selected from H and C₁₋₅ alkyl; or R¹ and R² together with the C atom to which they are attached combine to form a C₄₋₇ cycloalkyl ring or a 4-7 membered heterocyclic ring, both of which are optionally substituted on an available atom by one or more groups selected from C₁₋₂ alkyl and oxo;
n is 0 or 1; and
m is an integer in the range 1-5,000,000;
and,
component C is a moiety comprising one or more amine groups;
wherein core moiety A comprises one or more moieties independently selected from the group consisting of alkyl, spiroalkyl, aryl, heteroaryl, alkyl-aryl, a porphyrin, a polymer and a macrocycle, wherein alkyl, spiroalkyl, aryl, heteroaryl and alkyl-aryl are optionally substituted.

59. The chemical entity of claim 58, wherein at least a proportion of the thiol (—SH) groups in the chemical entity have been converted to S-nitrosothiol (—SNO) groups.

60. The chemical entity according to claim 58 or claim 59, wherein:
m is 1 or 2 and the core moiety A comprises an alkyl group or is an alkyl group, or
m is 2 and the core moiety A comprises a C₁₋₁₀ alkylene moiety; or
m is 2 and core moiety A is: 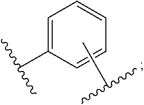
or
m is 3 and core moiety A is: 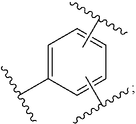
for example
m is 2 and core moiety A is: 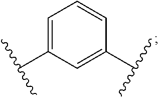 [SMILES: *c1cccc(*)c1]
or
m is 3 and core moiety A is:

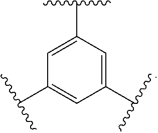 [SMILES: *c1cc(*)cc(*)c1]

61. The chemical entity according to claim 58 to 60, wherein linker L is absent; or wherein n is 0; or wherein n is 1; or wherein R¹ and R² are independently selected from the group consisting of H and C₁₋₅ alkyl.

62. The chemical entity according to any one of claims 58, 59 or 61, wherein m is 2, 3 or 4; or wherein m is 5-10; or wherein m is 5,000-100,000; or wherein m is 2-5,000,000.

63. The chemical entity according to any one of claims 58 to 62, wherein component B is of Formula (Ia): 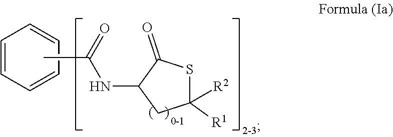
or component B is of Formula (Iaa): 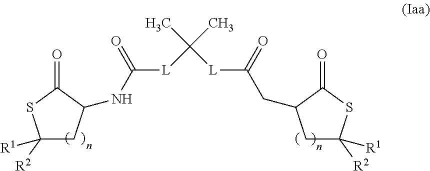
where L, n, R¹ and R² are as defined in any one of claims 1 to 6; for example, wherein L is absent and component B is of Formula (Iab): 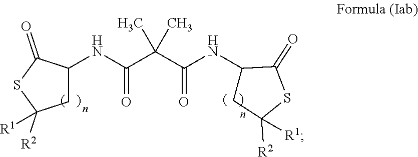
or wherein L is absent, n is 0 and component B is of Formula (Iac):

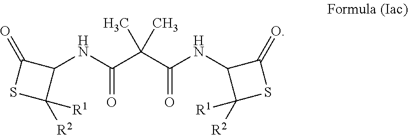

64. The chemical entity according to any one of claims 58 to 63, wherein component C is a single monomeric compound; or wherein component C is a polymer selected from a straight chain polymer, a branched or hyperbranched polymer or a dendrimer; or wherein the polymer is formed from a single monomer; or wherein the polymer is formed from two or more monomers; or wherein component C is a mixture of two or more compounds.

65. The chemical entity according to claim 64, wherein the monomer units forming the polymer are alkylene oxide units or mixtures of alkylene oxide units, and/or the monomer units forming the polymer have pendant amine groups.

66. The chemical entity according to claim 64, wherein component C is selected from the group consisting of aniline, benzylamine, morpholine, diethylamine, dibutylamine, 1-ethylpropylamine, 2-aminopentane, piperidine, 4-methylpiperidine, pyrrolidine, t-butylamine, t-amylamine, propylamine, butylamine, amylamine, isopentylamine, hexylamine, heptylamine, octylamine, nonylamine, decylamine, dodecylamine, hexadecylamine, octadecylamine, 4-methylaniline, 2-fluorobenzylamine, 3-fluorobenzylamine, 4-fluorobenzylamine, 3-(trifluoromethyl)benzylamine, 4-(trifluoromethyl)benzylamine and 3,5-is(trifluoromethyl)benzylamine.

67. The chemical entity according to claim 64, wherein component C is a straight chain polymer comprising two amine groups, one at each terminus of the polymer chain; optionally wherein component C comprises of one or two polyalkylene oxide polymers, wherein each polymer has an amino group at each terminus; or
wherein: component C comprises a single straight chain polyalkylene oxide polymer having an amino group at each terminus; or
component C comprises a mixture of two, straight chain polyalkylene oxide polymers, wherein each polymer has an amino group at each terminus.

68. The chemical entity according to any one of claims 58 to 67, wherein component C is a compound of Formula (X) or a mixture of two or more compounds of Formula (X): 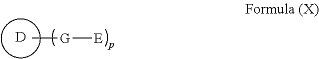
wherein,
D is a core moiety;
G is an optional linker;
E is NH₂; and
p is an integer of at least 1.

69. The chemical entity according to claim 68, wherein D is a polymer comprising one or more C₂₋₄ alkylene oxide units, for example ethylene oxide units and/or propylene oxide units; and/or wherein component C is a single compound of Formula (X), wherein the average number of alkylene oxide units in the core moiety D is from 2 to 20;
or wherein component C is a mixture of a first compound of Formula (X) and a second compound of Formula (X), wherein, in the first compound of Formula (X), D is a polymer which comprises an average number of alkylene oxide units from 2 to 20 and in the second compound of Formula (X), D is a polymer which comprises an average number of alkylene oxide units from 11 to 100; wherein optionally, in the compound of Formula (X), the alkylene oxide units are all the same; wherein optionally the compound of Formula (X) comprises two or more types of alkylene oxide unit; and wherein optionally the linker G is absent and/or p is 2.

70. The chemical entity according to claim 68 or claim 69, wherein D is a polyalkylsiloxane moiety substituted with primary and/or secondary amine groups.

71. The chemical entity according to any one of claims 58 to 70, wherein the chemical entity comprises: 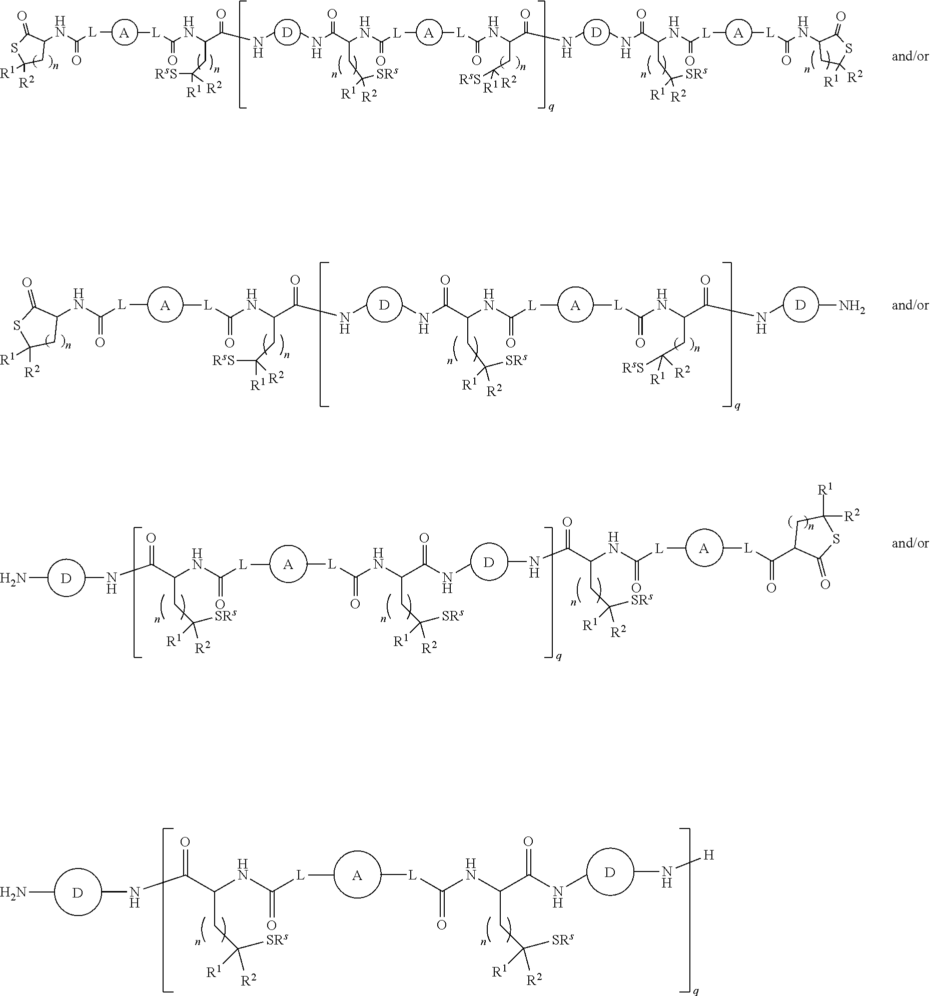
wherein,
A, n, R¹, R², and D are as defined in any one of claims 58 to 67;
R^{s} is H or N═O; and
q is 10 or 1000; or
the chemical entity comprises: 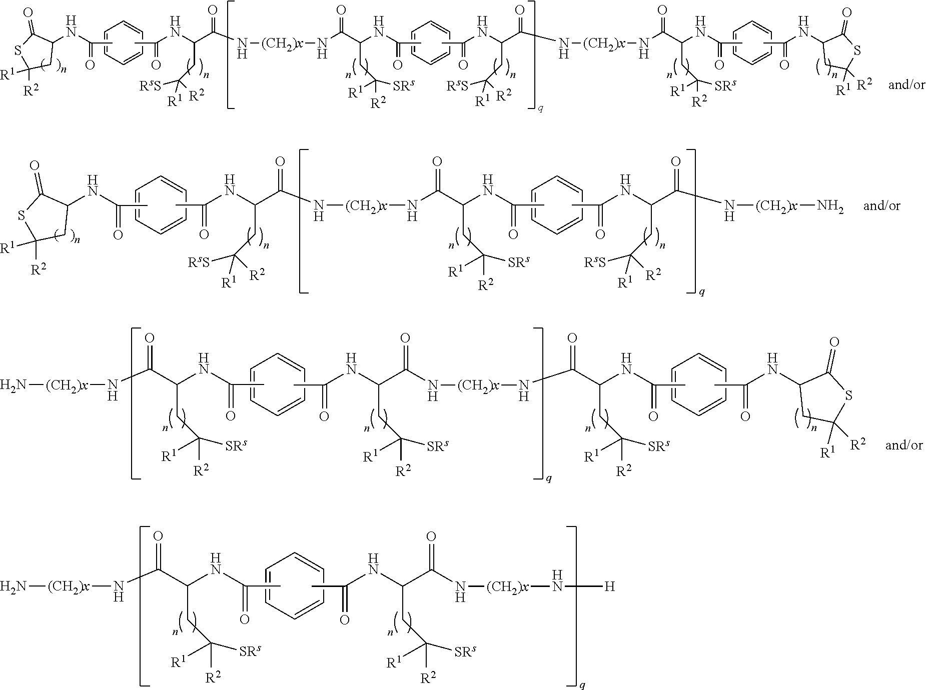
wherein n is as defined in any one of claims 58 to 70;
x is 1-10;
R^{s} is H or N═O; and
q is 10 to 1000; or
the chemical entity comprises: 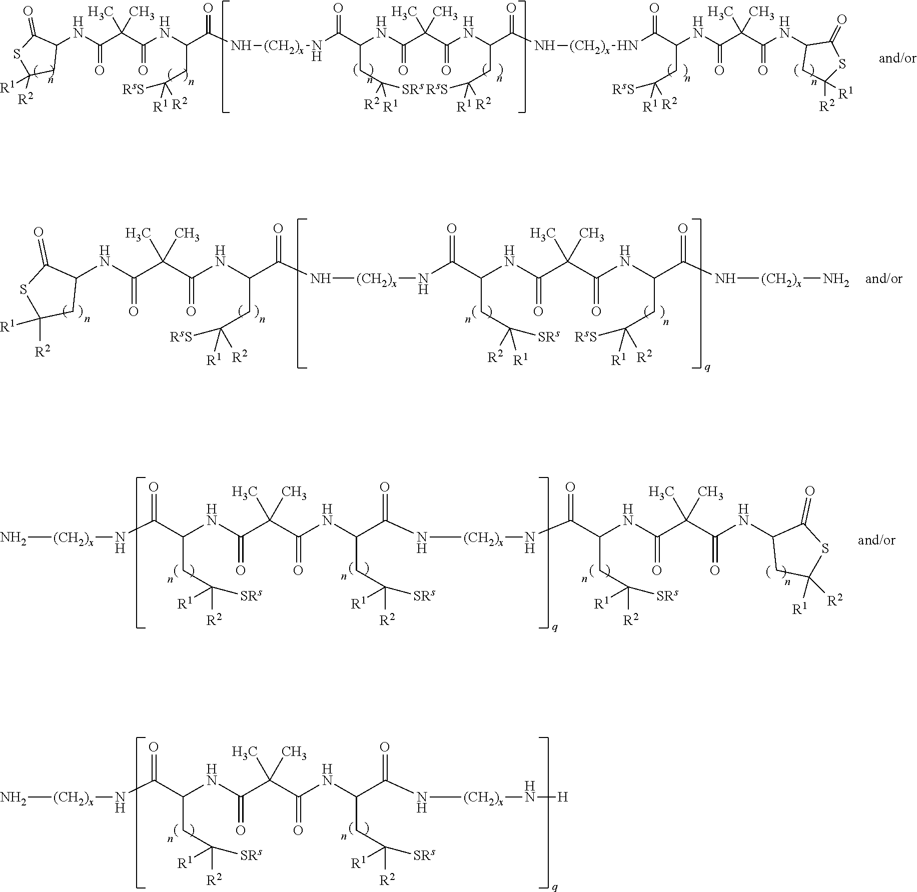
x is 1-10;
R^{s} is H or N═O; and
q is 100-20,000; or
the chemical entity comprises: 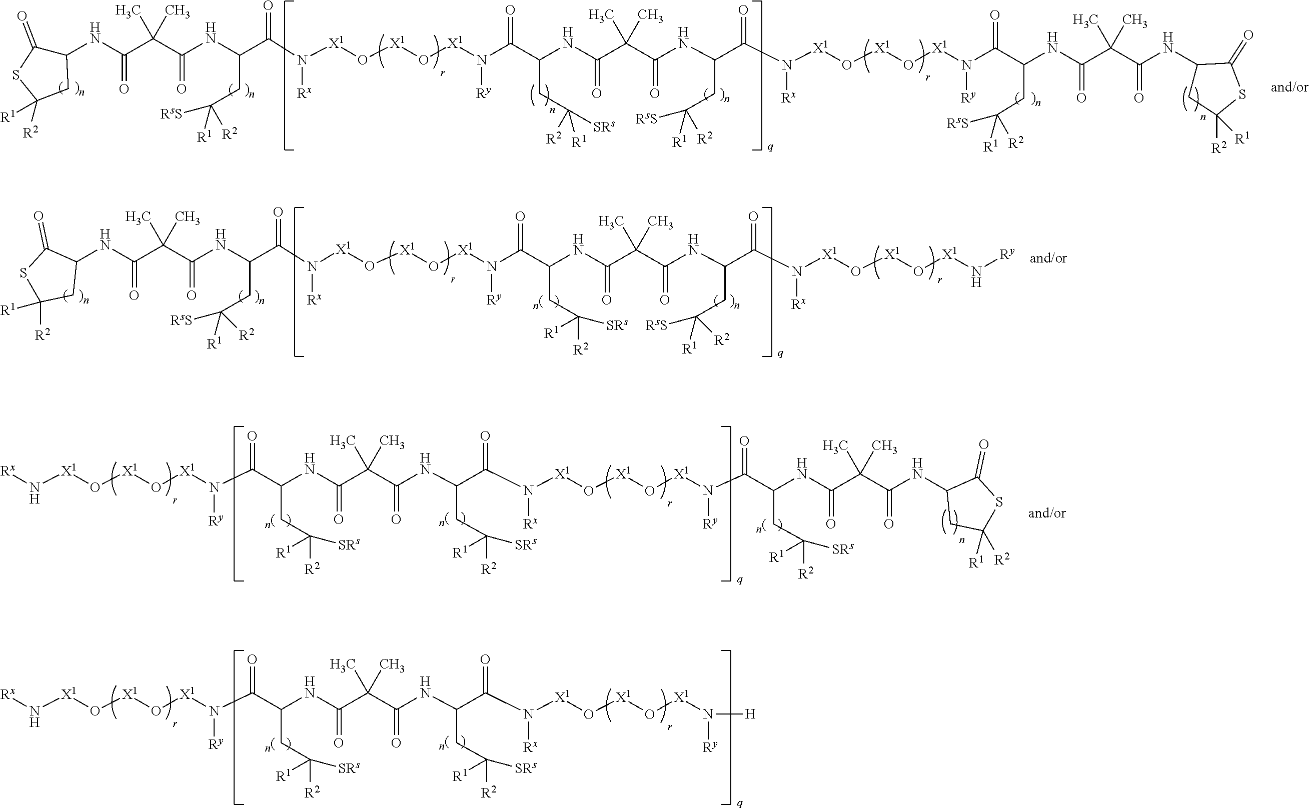
wherein n, X¹ and r are as defined in any one of claims 58 to 70;
R^{s} is H or N═O;
each of R^{x} and R^{y} is H or C₁₋₆ alkyl; and
q is 10 to 1000; or
the chemical entity comprises: 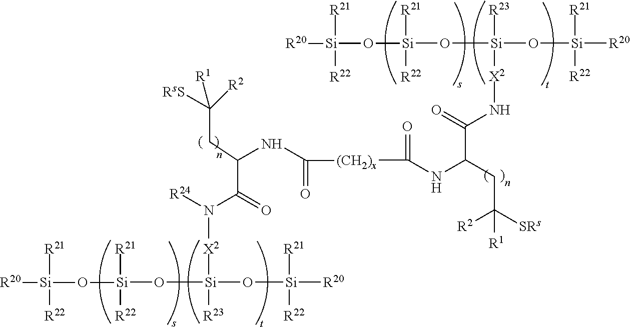
wherein R¹, R², R²⁰, R²¹, R²², R²³, n, X², s and t are as defined in any one of claims 58 to 70;
R^{s} is H or N═O; and
x is 1-10.

72. A method of forming a coating on a surface, wherein said method comprises the step of:
(a) contacting the surface with the chemical entity according to any of claims 58 to 71;
optionally further comprising step (b) of converting at least a proportion of the thiol (—SH) groups in the chemical entity to S-nitrosothiol (—SNO) groups.
